(19) United States
(12) Patent Application Publication
(10) Pub. No.: US 2008/0249081 A1
(43) Pub. Date: Oct. 9, 2008

(54) COMPOUNDS FOR THE TREATMENT OF PAIN AND SCREENING METHODS THEREFOR

(76) Inventors: Roger Olsson, Bunkeflostrand (SE); Anne Eeg Knapp, Frederiksberg (DK); Jorgen Eskildsen, Copenhagen (DK)

Correspondence Address: Biotechnology Law Group;c/o Portfolioip, P.O. Box 52050, Minneapolis, MN, 55402, US

(21) Appl. No.: 11/923,460

(22) Filed: Oct. 24, 2007

Related U.S. Application Data
(60) Provisional application No. 60/862,685, filed on Oct. 24, 2006.

(51) Int. Cl. A61K 31/5513; C07D 403/08; A61K 31/496; C07D 401/12; A61K 31/5377; C12N 5/06; G01N 33/567; G01N 33/566; C07K 14/705; C07D 413/12; A61K 31/4439; A61P 25/02

(52) U.S. Cl. 514/218; 544/370; 514/254.06; 514/338; 546/273.4; 540/575; 514/234.5; 544/139; 530/350; 435/375; 436/501; 435/7.21

(57) ABSTRACT

Disclosed herein are benzoimidazole compounds; pharmaceutical compositions comprising a therapeutically effective amount of the same; methods of modulating the activity of an MrgX1 or an MrgX2 receptor using the same; and methods of alleviating acute, chronic and neuropathic pain in a subject using the same. Also disclosed are methods of identifying a benzoimidazole compound that modulates the activity of an MrgX1 or an MrgX2 receptor; methods of identifying a benzoimidazole compound effective for the treatment of pain.

### RELATED APPLICATIONS

The present application claims priority to the U.S. Provisional Patent Application Ser. No. 60/862,685, filed on Oct. 24, 2006, by Roger Olsson et al., and entitled “COMPOUNDS FOR THE TREATMENT OF PAIN AND SCREENING METHODS THEREFOR”, the entire disclosure of which is incorporated herein by reference in its entirety, including any drawings.

### FIELD OF THE INVENTION

The present invention relates to compounds that are useful for the treatment and prevention of pain and to methods of screening for them.

### BACKGROUND

Opiates are currently the most extensively used compounds for the clinical treatment of pain (Reisine and Pasternak, 1996). The opiates, however, have a number of side effects that limit their therapeutic use. They can cause respiratory depression and nausea and, of course, they are addictive. The side effects are primarily due to their activity at central sites in the brain. It is believed that compounds that interact with targets in peripheral pain pathways rather than the central nervous system may be capable of reducing pain with minimal side effects. The dorsal root ganglia (DRG) constitute such a target.

The DRG contain afferent neurons known as nociceptive neurons that respond to acute and chronic pain stimuli in the peripheral organs and transmit signals to the central nervous system causing the sensation of pain. Drugs that target nociceptive sensory neurons in the DRG could potentially block pain transmission with the desired minimal side effects including, in particular, physical dependency.

Recent studies have identified a family of G-protein coupled receptors (GPCRs) known as sensory neuron specific GPCRs (SNSRs) that are primarily expressed in DRG nociceptive neurons. These receptors are structurally similar to the Mas oncogene, which is also a GPCR and are also referred to as Mas related genes (Mrgs) (Dong et al., 2001; Zylka et al., 2003). In addition, certain endogenous peptides have been identified that interact with SNSRs to produce analgesia in rodents (Hong et al., 2004; Lembo et al., 2002; Han et al., 2002, Robas et al., 2003; Grazzini et al., 2004). For example, MrgA1 and MrgC11 are activated by RF-amide related peptides (Han et al., 2002) which blocks pain transmission when administered intrathecally (Panula et al. 1996, 1999). Similarly, MrgX1, which in humans is only expressed in DRG, is potently stimulated in vitro by the opioid peptide bovine adrenal medulla peptide 22 (BAM22). BAM22 is believed to induce analgesia through mechanisms independent of opiate receptor stimulation (Hong et al. 2004). These studies and others suggest that the Mrgs may be useful targets for the development of novel analgesics. Unfortunately, no homologues of MrgX receptors exist in rodents (see Dong et al, 2001) rendering them ineffective as animal models for testing.

### SUMMARY OF THE INVENTION

Disclosed herein is a compound of Formula III:

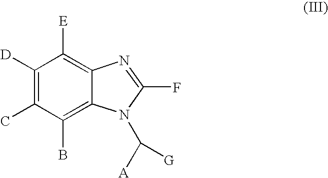

or a pharmaceutically acceptable salt or prodrug thereof, and pharmaceutical compositions comprising a therapeutically effective amount of the same.

Also disclosed are methods of modulating the activity of an MrgX1 or an MrgX2 receptor, comprising contacting the MrgX1 or the MrgX2 receptor with a compound of Formula I; and comparing the activity of the receptor after the contacting to the activity of the receptor before the contacting.

Further disclosed are methods of alleviating acute, chronic and neuropathic pain in a subject, comprising identifying a subject in need thereof, and administering to the subject a therapeutically effective amount of a compound of Formula I.

Also disclosed are methods of identifying a compound that modulates the activity of an MrgX1 or an MrgX2 receptor, comprising contacting the MrgX1 or the MrgX2 receptor with a plurality of compounds of Formula I one at a time; comparing the activity of the receptor after the contacting with each compound of Formula I to the activity of the receptor before the contacting; and selecting a compound of Formula I that changes the activity of the receptor after the contacting.

In addition, disclosed are methods of identifying a compound effective for the treatment of pain, comprising contacting a compound of Formula I with a receptor selected from the group consisting of a human MrgX1 receptor, a simian MrgX1 receptor, a human MrgX2 receptor, and a simian MrgX2 receptor; comparing the activity of the receptor after the contacting with each compound of Formula I to the activity of the receptor before the contacting; and selecting a compound of Formula I that changes the activity of the receptor after the contacting.

### BRIEF DESCRIPTION OF THE DRAWINGS

FIG. 1 is a series of graphs showing the activity of MrgX1-selective agonists in Receptor Selection and Amplification Technology (RSAT®) assays. Shown are the responses (in absorbance units) of human (Hum) and rhesus monkey (Mky) MrgX1 and MrgX2 receptors to the indicated log of the concentrations of compounds 1 and 3.

FIG. 2 is a series of graphs showing the activity of MrgX1-selective agonists in calcium mobilization assays. Shown are the responses, normalized to the response to the reference peptide BAM22 (100%, not shown), of human MrgX1 receptors to the indicated log of the concentrations of compounds 1 and 3.

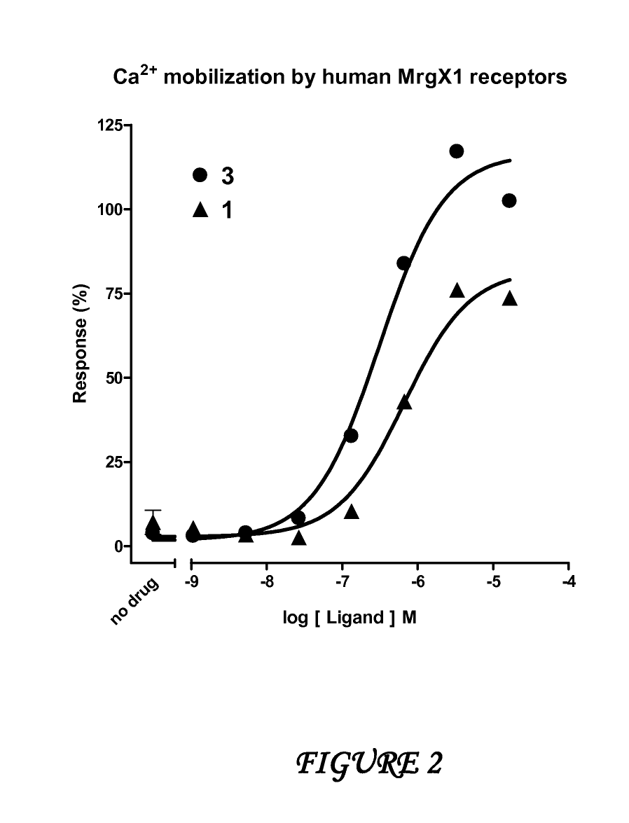

### DETAILED DESCRIPTION OF THE INVENTION

### I. Compounds

The term “pharmaceutically acceptable salt” refers to a formulation of a compound that does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3.sup.rd Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Whenever a group of this invention is described as being “optionally substituted” that group may be unsubstituted or substituted with one or more of the substituents described for that group. Likewise, when a group is described as being “unsubstituted or substituted,” if substituted, the substituent may be selected from the same group of substituents. Unless otherwise indicated, when a substituent is deemed to be “optionally substituted,” or “substituted” it is meant that the substitutent is a group that may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (hetereoalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. The protecting groups that may form the protective derivatives of the above substituents are known to those of skill in the art and may be found in references Greene and Wuts, Protective Groups in Organic Synthesis, 3^{rd} Ed., John Wiley & Sons, New York, N.Y., 1999, which is hereby incorporated by reference in its entirety.

As used herein, “Cₘ-Cₙ” in which “m” and “n” are integers refers to the number of carbon atoms in an alkyl, alkenyl or alkynyl group or the number of carbon atoms in the ring of a cycloalkyl, cycloalkenyl, or aryl group. That is, the alkyl, alkenyl, alkynyl, ring of the cycloalkyl, ring of the cycloalkenyl, or of the aryl can contain from “m” to “n”, inclusive, carbon atoms. Thus, for example, a “C₁-C₄ alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)—, and (CH₃)₃CH—. If no “m” and “n” are designated with regard to an alkyl, alkenyl, alkynyl, cycloalkyl or cycloalkenyl group, the broadest range described in these definitions is to be assumed.

As used herein, “alkyl” refers to a straight or branched chain fully saturated (no double or triple bonds) hydrocarbon (all carbon) group. An alkyl group of this invention may comprise from 1-20 carbon atoms, that is, “m”=1 and “n”=20, designated as a “C₁ to C₂₀ alkyl.” It is presently preferred that “m”=1 and “n”:=12 (C₁ to C₁₂ alkyl). It is presently more preferred that “m”=1 and “n”=6 (C₁ to C₆ alkyl). Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl and dodecyl.

An alkyl group of this invention may be substituted or unsubstituted. When substituted, the substituent group(s) is(are) one or more group(s) independently selected from cycloalkyl, aryl, heteroaryl, heteroalicyclyl, hydroxy, alkoxy, aryloxy, mercapto, alkylthio, arylthio, cyano, halo, oxo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, —NR^{a}R^{b}, protected hydroxyl, protected amino, protected carboxy and protected amido groups.

Examples of substituted alkyl groups include, without limitation, 2-oxo-prop-1-yl, 3-oxo-but-1-yl, cyanomethyl, nitromethyl, chloromethyl, hydroxymethyl, tetrahydropyranyloxymethyl, m-trityloxymethyl, propionyloxymethyl, aminomethyl, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichlorobutyl, 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, N-acetyl-2-aminoethyl, N-benzoyl-1-aminoethyl and N-acetyl-1-aminoethyl.

As used herein, “alkenyl” refers to an alkyl group that contains in the straight or branched hydrocarbon chain one or more double bonds. Examples of alkenyl groups include, without limitation, vinyl (CH₂═CH—), allyl (CH₃CH═CH₂—), 1-propenyl, 2-propenyl, 1-butenyl, 2-butenyl; 1-pentenyl, 2-pentenyl, 3-pentenyl, 4-pentenyl, 3-methyl-1-butenyl, and the various isomers of hexenyl, heptenyl, octenyl, nonenyl, decenyl undecenyl and dodecenyl.

An alkenyl group of this invention may be unsubstituted or substituted. When substituted, the substituent(s) may be selected from the same groups disclosed above with regard to alkyl group substitution. Examples of substituted alkenyl groups include, without limitation, styrenyl, 3-chloro-propen-1-yl, 3-chloro-buten-1-yl, 3-methoxy-propen-2-yl, 3-phenyl-buten-2-yl and 1-cyano-buten-3-yl.

As used herein, “alkynyl” refers to an alkyl group that contains in the straight or branched hydrocarbon chain one or more triple bonds.

An alkynyl group of this invention may be unsubstituted or substituted. When substituted, the substituent(s) may be selected from the same groups disclosed above with regard to alkyl group substitution.

As used herein, “cycloalkyl” refers to a completely saturated (no double bonds) hydrocarbon ring. Cycloalkyl groups of this invention may range from C₃ to C₈. A cycloalkyl group may be unsubstituted or substituted. If substituted, the substituent(s) may be selected from those indicated above with regard to substitution of an alkyl group. The “cycloalkyl” group can be made up of two or more fused rings (rings that share two adjacent carbon atoms). When the cycloalkyl is a fused ring system, then the ring that is connected to the rest of the molecule is a cycloalkyl as defined above. The other ring(s) in the fused ring system may be a cycloalkyl, a cycloalkenyl, an aryl, a heteroaryl, or a heteroalicyclic.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). A cycloalkenyl group of this invention may unsubstituted or substituted. When substituted, the substituent(s) may be selected from the same groups disclosed above with regard to alkyl group substitution. The “cycloalkenyl” group can be made up of two or more fused rings (rings that share two adjacent carbon atoms). When the cycloalkenyl is a fused ring system, then the ring that is connected to the rest of the molecule is a cycloalkenyl as defined above. The other ring(s) in the fused ring system may be a cycloalkyl, a cycloalkenyl, an aryl, a heteroaryl, or a heteroalicyclic.

The term “alkylene” refers to an alkyl group, as defined here, which is a biradical and is connected to two other moieties. Thus, methylene (—CH₂—), ethylene (—CH₂CH₂—), propylene (—CH₂CH₂CH₂—), isopropylene (—CH₂—CH(CH₃)—), and isobutylene (—CH₂—CH(CH₃)—CH₂—) are examples, without limitation, of an alkylene group. Similarly, the term “cycloalkylene” refers to a cycloalkyl group, as defined here, which binds in an analogous way to two other moieties. If the alkyl and cycloalkyl groups contain unsaturated carbons, the terms “alkenylene” and “cycloalkenylene” are used.

As used herein, “acyl” refers to an “RC(═O)O—” Examples of acyl groups include, without limitation, formyl, acetyl, propionyl, butyryl, pentanoyl, pivaloyl, hexanoyl, heptanoyl, octanoyl, nonanoyl, decanoyl, undecanoyl, dodecanoyl and benzoyl. Presently preferred acyl groups are acetyl and benzoyl.

An acyl group of this invention may be unsubstituted or substituted. When substituted, the substituent(s) may be selected from the same groups disclosed above with regard to alkyl group substitution. Example of substituted acyl groups include, without limitation, 4-phenylbutyroyl, 3-phenylbutyroyl, 3-phenylpropanoyl, 2-cyclohexanylacetyl, cyclohexanecarbonyl, 2-furanoyl and 3-dimethylaminobenzoyl.

As used herein, “aryl” refers to a carbocyclic (all carbon) ring that has a fully delocalized pi-electron system. The “aryl” group can be made up of two or more fused rings (rings that share two adjacent carbon atoms). When the aryl is a fused ring system, then the ring that is connected to the rest of the molecule has a fully delocalized pi-electron system. The other ring(s) in the fused ring system may or may not have a fully delocalized pi-electron system. Examples of aryl groups include, but are not limited to, benzene, naphthalene and azulene.

As used herein, “heteroaryl” refers to a ring that contains one or more heteroatoms selected from the group consisting of nitrogen, oxygen and sulfur in the ring and that has a fully delocalized pi-electron system. The “heteroaryl” group can be made up of two or more fused rings (rings that share two adjacent carbon atoms). When the heteroaryl is a fused ring system, then the ring that is connected to the rest of the molecule has a fully delocalized pi-electron system. The other ring(s) in the fused ring system may or may not have a fully delocalized pi-electron system. Examples of heteroaryl rings include, but are not limited to, furan, thiophene, phthalazinone, pyrrole, oxazole, thiazole, imidazole, pyrazole, isoxazole, isothiazole, triazole, thiadiazole, pyran, pyridine, pyridazine, pyrimidine, pyrazine and triazine.

As used herein, “heterocycloalkyl,” “heteroalicyclic,” or “heteroalicyclyl” refers to a ring having in the ring system one or more heteroatoms independently selected from nitrogen, oxygen and sulfur. The ring may also contain one or more double bonds provided that they do not form a fully delocalized pi-electron system in the rings. Heteroalicyclyl groups of this invention may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethanesulfonamido. The “heterocycloalkyl” group can be made up of two or more fused rings (rings that share two adjacent carbon atoms). When the heterocycloalkyl is a fused ring system, then the ring that is connected to the rest of the molecule is a heterocycloalkyl as defined above. The other ring(s) in the fused ring system may be a cycloalkyl, a cycloalkenyl, an aryl, a heteroaryl, or a heteroalicyclic.

As used herein, “phenylalkyl” refers to a phenyl ring covalently bonded to an alkyl group as defined herein. Examples, without limitation, of phenylalkyl groups include, without limitation, benzyl, 2-phenylethyl, 1-phenylpropyl, 4-phenylhexyl, 3-phenylamyl and 3-phenyl-2-methylpropyl. Presently preferred phenylalkyl groups are those wherein the phenyl group is covalently bonded to one of the presently preferred alkyl groups. A phenyl alkyl group of this invention may be unsubstituted or substituted. Examples of substituted phenylalkyl groups include, without limitation, 2-phenyl-1-chloroethyl, 2-(4-methoxyphenyl)ethyl, 4-(2,6-dihydroxy phenyl)hexyl, 2-(5-cyano-3-methoxyphenyl)pentyl, 3-(2,6-dimethylphenyl)propyl, 4-chloro-3-aminobenzyl, 6-(4-methoxyphenyl)-3-carboxy(n-hexyl), 5-(4-aminomethylphenyl)-3-(aminomethyl)pentyl and 5-phenyl-3-oxo-pent-1-yl.

As used herein, “heteroarylalkyl” and “heteroalicyclylalkyl” refer to a heteroaryl or a heteroalicyclyl group covalently bonded to an alkyl group, as defined herein. Examples of such groups include, without limitation, 2-pyridylethyl, 3-pyridylpropyl, 4-furylhexyl, 3-piperazylamyl and 3-morpholinylbutyl. Presently preferred heteroarylalkyl and heteroalicyclylalkyl groups are those in which a presently preferred heteroaryl or heteroalicyclyl group is covalently bonded to a presently preferred alkyl group as disclosed herein.

As used herein, “phenyl” refers to a 6-member aryl group. A phenyl group may be unsubstituted or substituted. When substituted the substituent(s) is/are one or more, preferably one or two, group(s) independently selected from the group consisting of halogen, hydroxy, protected hydroxy, cyano, nitro, alkyl, alkoxy, acyl, acyloxy, carboxy, protected carboxy, carboxymethyl, protected carboxymethyl, hydroxymethyl, protected hydroxymethyl, —NR^{a}R^{b} wherein R^{a} and R^{b} are as defined above but in addition R^{a} may be an amino protecting group as defined herein, carboxamide, protected carboxamide, N-alkylcarboxamide, protected N-alkylcarboxamide, N,N-dialkylcarboxamide, trifluoromethyl, N-alkylsulfonylamino, N-(phenylsulfonyl)amino and phenyl (resulting in the formation of a biphenyl group).

Examples of substituted phenyl groups include, without limitation, 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 and 4-fluorophenyl, 2, 3 or 4-hydroxyphenyl, 2,4-dihydroxyphenyl, the protected-hydroxy derivatives thereof, 2, 3 or 4-nitrophenyl; 2, 3 or 4-cyanophenyl; 2, 3 or 4-methylphenyl, 2,4-dimethylphenyl, 2, 3 or 4-(iso-propyl)phenyl, 2, 3 or 4-ethylphenyl, 2, 3 or 4-(n-propyl)phenyl, 2,6-dimethoxyphenyl, 2, 3 or 4-methoxyphenyl, 2, 3 or 4-ethoxyphenyl, 2, 3 or 4-(isopropoxy)phenyl, 2, 3 or 4-(t-butoxy)phenyl, 3-ethoxy-4-methoxyphenyl; 2, 3 or 4-trifluoromethylphenyl; 2, 3 or 4-carboxyphenyl or 2,4-di(protected carboxy)phenyl; 2,3, or 4-(protected hydroxymethyl)phenyl or 3,4-di(hydroxymethyl)phenyl; 2, 3 or 4-(aminomethyl)phenyl or 2,4-(protected aminomethyl)phenyl; and 2, 3 or 4-(N-(methylsulfonylamino))phenyl.

As used herein, “phenylalkoxy” refers to a “phenylalkyl-O—” group with “phenyl” and “alkyl” as defined herein. A phenylalkoxy group of this invention may be substituted or unsubstituted on the phenyl ring, in the alkyl group or both. Examples of phenylalkoxy groups include, without limitation, 2-(4-hydroxyphenyl)ethoxy, 4-(4-methoxyphenyl)butoxy, (2R)-3-phenyl-2-amino-propoxy, (2S)-3-phenyl-2-amino-propoxy, 2-indanoxy, 6-phenyl-1-hexanoxy, cinnamyloxy, 2-phenyl-1-propoxy and 2,2-dimethyl-3-phenyl-1-propoxy.

As used herein, “halo” and “halogen” refer to the fluoro, chloro, bromo or iodo atoms. Presently preferred halogens are chloro and fluoro.

As used herein, “amino protecting group” refers to a group commonly employed to keep (i.e., to “block” or “protect”) an amino group from reacting with a reagent while it reacts with an intended target functional group of a molecule.

As used herein, a “protected carboxamide” refers to a carboxamide in which the nitrogen is substituted with an amino protecting group.

Examples of amino protecting groups include, without limitation, formyl (“For”), trityl, phthalimido, trichloroacetyl, chloroacetyl, bromoacetyl, iodoacetyl groups, t-butoxycarbonyl (“Boc”), 2-(4-biphenylyl)propyl-2-oxycarbonyl (“Bpoc”), 2-phenylpropyl-2-oxycarbonyl (“Poc”), 2-(4-xenyl)isopropoxycarbonyl, 1,1-diphenylethyl-1-oxycarbonyl, 1,1-diphenylpropyl-1-oxycarbonyl, 2-(3,5-dimethoxyphenyl)propyl-2-oxycarbonyl (“Ddz”), 2-(p-toluoyl)propyl-2-oxycarbonyl, cyclopentanyloxycarbonyl, 1-methylcyclopentanyloxycarbonyl, cyclohexanyloxy-carbonyl, 1-methylcyclohexanyloxycarbonyl, 2-methylcyclohexanyloxycarbonyl, 2-(4-toluoylsulfonyl)-ethoxycarbonyl, 2-(methylsulfonyl)ethoxycarbonyl, 2-(triphenylphosphino)-ethoxycarbonyl, 9-fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyl-oxycarbonyl, 2,2,2-trichloroethoxycarbonyl, 2-ethynyl-2-propoxycarbonyl, cyclopropyl-methoxycarbonyl, isobornyloxycarbonyl, 1-piperidyloxycarbonyl, benzyloxycarbonyl (“Cbz”), 4-phenylbenzyloxycarbonyl, 2-methylbenzyloxy-carbonyl, -2,4,5-tetramethylbenzyloxycarbonyl (“Tmz”), 4-methoxybenzyloxy-carbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyl-oxycarbonyl, 4-bromobenzyloxycarbonyl, 3-bromobenzyloxycarbonyl, 4-nitrobenzyloxy-carbonyl, 4-cyanobenzyloxycarbonyl, 4-(decyloxy)benzyloxycarbonyl, benzoylmethylsulfonyl, dithiasuccinoyl (“Dts”), 2-(nitro)phenylsulfenyl (“Nps”), and diphenyl-phosphine oxide. The species of amino-protecting group employed is not critical so long as the derivatized amino group is stable to the conditions of the subsequent reaction(s) and can be removed at the appropriate point without disrupting the remainder of the molecule. Presently preferred amino-protecting groups are Boc, and Fmoc. Descriptions of these and other amino-protecting groups may be found in T. W. Greene and P. G. M. Wuts, “Protective Groups in Organic Synthesis,” 2nd ed., John Wiley and Sons, New York, N.Y., 1991, Chapter 7, M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984.

As used herein, the term “carboxy protecting group” refers to a labile ester commonly used to block or protect a carboxylic acid while reactions are carried out on other functional groups on the compound. Examples of carboxy protecting groups include, without limitation, t-butyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxytrityl, 4,4′,4″-trimethoxytrityl, 2-phenylpropyl, trimethylsilyl, t-butyldimethylsilyl, phenacyl, 2,2,2-trichloroethyl, -(trimethylsilyl)ethyl, -(di(n-butyl)methylsilyl)ethyl, p-toluenesulfonylethyl, 4-nitrobenzylsulfonylethyl, allyl, cinnamyl, and 1-(trimethylsilylmethyl)-propenyl. The ester employed is not critical so long as it is stable to the conditions of subsequent reaction(s) and can be removed at the appropriate point without disrupting the remainder of the molecule. Further examples of carboxy-protecting groups are found in E. Haslam, “Protective Groups in Organic Chemistry,” J. G. W. McOmie, Ed., Plenum Press, New York, N.Y., 1973, Chapter 5, and T. W. Greene and P. G. M. Wuts, “Protective Groups in Organic Synthesis,” 2nd ed., John Wiley and Sons, New York, N.Y., 1991, Chapter 5.

As used herein, a “hydroxyl protecting group” refers to a readily cleavable group that replaces the hydrogen of the hydroxyl group, such as, without limitation, tetrahydropyranyl, 2-methoxypropyl, 1-ethoxyethyl, methoxymethyl, 2-methoxyethoxymethyl, methylthiomethyl, t-butyl, t-amyl, trityl, 4-methoxytrityl, 4,4′-dimethoxytrityl, 4,4′,4″-trimethoxytrityl, benzyl, allyl, trimethylsilyl, (t-butyl)dimethylsilyl, and 2,2,2-trichloroethoxycarbonyl. The species of hydroxyl protecting groups is not critical so long as the derivatized hydroxyl group is stable to the conditions of subsequent reaction(s) and can be removed at the appropriate point without disrupting the remainder of the molecule. Further examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2nd ed., John Wiley and Sons, New York, N.Y., 1991, Chapters 2 and 3.

As used herein, “alkylthio” refers to an “alkyl-S—” group, with alkyl as defined above. Examples of alkylthio group include, without limitation, methylthio, ethylthio, n-propylthio, isopropylthio, n-butylthio and t-butylthio.

As used herein, “alkylsulfinyl” refers to an “alkyl-SO—” group, with alkyl as defined above. Examples of alkylsulfinyl groups include, without limitation, methylsulfinyl, ethylsulfinyl, n-propylsulfinyl, isopropylsulfinyl, n-butylsulfinyl and sec-butylsulfinyl.

As used herein, “alkylsulfonyl” refers to an “alkyl-SO₂—” group. Examples of alkylsulfonyl groups include, without limitation, methylsulfonyl, ethylsulfonyl, n-propylsulfonyl, isopropylsulfonyl, n-butylsulfonyl, and t-butylsulfonyl.

As used herein, “phenylthio,” “phenylsulfinyl,” and “phenylsulfonyl” refer to a “phenyl-S—,” “phenyl-SO—,” and “phenyl-SO₂—” group, phenyl as defined herein.

As used herein, “alkylaminocarbonyl” refers to an “alkylNHC(═O)—” group, with alkyl as defined herein. Examples of alkylaminocarbonyl groups include, without limitation, methylaminocarbonyl, ethylaminocarbonyl, propylaminocarbonyl and butylaminocarbonyl. Examples of substituted alkylaminocarbonyl include, without limitation, methoxymethyl-aminocarbonyl, 2-chloroethylaminocarbonyl, 2-oxopropylaminocarbonyl and 4-phenylbutylaminocarbonyl.

As used herein, “alkoxycarbonyl” refers to an “alkyl-OC(═O)—” group, with alkyl as defined above.

As used herein, “phenylaminocarbonyl” refers to a “phenyl-NHC(═O)—” group, with phenyl as defined above. Examples of substituted phenylaminocarbonyl groups include, without limitation, 2-chlorophenyl-aminocarbonyl, 3-chlorophenylaminocarbonyl, 2-nitorphenylaminocarbonyl, 4-biphenylaminocarbonyl, and 4-methoxyphenylaminocarbonyl.

As used herein, “alkylaminothiocarbonyl” refers to an “alkyl-NHC(═O)—” group, with alkyl as defined above. Examples of alkylaminothio-carbonyl groups include, without limitation, methylaminothiocarbonyl, ethylaminothiocarbonyl, propylaminothiocarbonyl and butylaminothiocarbonyl.

Examples of alkyl-substituted alkylaminothiocarbonyl groups include, without limitation, methoxymethylaminothiocarbonyl, 2-chloroethylaminothiocarbonyl, 2-oxopropylaminothiocarbonyl and 4-phenylbutylaminothiocarbonyl.

As used herein, “phenylaminothiocarbonyl” refers to a “phenyl-NHC(═S)—” group, with phenyl as defined above. Examples of phenylaminothiocarbonyl groups include, without limitation, 2-chlorophenylaminothiocarbonyl, 3-chlorophenyl-aminothiocarbonyl, 2-nitrophenylaminothiocarbonyl, 4-biphenylaminothiocarbonyl and 4-methoxyphenylaminothiocarbonyl.

As used herein, “carbamoyl” refers to an “—NCO—” group.

As used herein, “hydroxyl” refers to an “—OH” group.

As used herein, “cyano” refers to a “—C≡N” group.

As used herein, “nitro” refers to an “—NO₂” group.

An “O-carboxy” group refers to a “RC(═O)O—” group with R as defined above.

A “C-carboxy” group refers to a “—C(═O)OR” group with R as defined above.

An “acetyl” group refers to a CH₃C(═O)— group.

A “trihalomethanesulfonyl” group refers to an “X₃CSO₂—” group wherein X is a halogen.

An “isocyanato” group refers to an “—NCO” group.

A “thiocyanato” group refers to a “—CNS” group.

An “isothiocyanato” group refers to an “—NCS” group.

A “sulfinyl” group refers to an “—S(═O)—R” group with R as defined above.

An “S-sulfonamido” group refers to a “—SO₂NR” group with R as defined above.

An “N-sulfonamido” group refers to a “RSO₂NH—” group with R as defined above.

A “trihalomethanesulfonamido” group refers to an “X₃CSO₂NR—” group with X as halogen and R as defined above.

An “O-carbamyl” group refers to a “—OC(═O)—NR” group with R as defined above.

An “N-carbamyl” group refers to an “ROC(═O)NH—” group with R as defined above.

An “O-thiocarbamyl” group refers to a “—OC(═S)—NR” group with R as defined above.

“N-thiocarbamyl” group refers to an “ROC(═S)NH—” group with R as defined above.

A “C-amido” group refers to a “—C(═O)—NR^{a}R^{b} group with R^{a} and R^{b} as defined above.

An “N-amido” group refers to a RC(═O)NH— group with R as defined above.

The term “perhaloalkyl” refers to an alkyl group in which all the hydrogen atoms are replaced by halogen atoms.

As used herein, an “ester” refers to a “—C(O)OR^{a}” group with R^{a} as defined herein.

As used herein, an “amide” refers to a “—C(O)NR^{a}R^{b}” group with R^{a} and R^{b} as defined herein.

Any unsubstituted or monosubstituted amine group on a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999). Compounds containing any such converted hydroxyl, amino and/or carboxylic acid groups are within the scope of this invention.

As used herein, an “ether” refers to an “—C—O—C—” group wherein either or both carbons may independently be part of an alkyl, alkenyl, alkynyl, aryl, heteroaryl or heteroalicyclyl group.

As used herein, a “halogenated ether” refers to an ether in which the groups to either side of the oxygen are both alkyl substituted with halogen.

As used herein, “amino acid” refers to any one of the twenty naturally-occurring L-amino acids, to their non-natural D-enantiomers, to non-naturally occurring amino acids such as, without limitation, norleucine (“Nle”), norvaline (“Nva”), L- or D-naphthalanine, ornithine (“Orn”), homoarginine (homoArg) and to other amino acids well-known in the peptide art such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill.

Amino acids are referred to herein by their full chemical names or by their three letter codes, which are well-known to those skilled in the art. Unless the chirality of an amino acid is specifically designated or the amino acid is expressly stated to be a naturally occurring (i.e., L-) amino acid, the amino acid may be D or L or a racemic mixture of the two.

As used herein, a “functionalized resin” refers to any resin to which functional groups have been appended. Such functionalized resins are well-known to those skilled in the art and include, without limitation, resins functionalized with amino, alkylhalo, formyl or hydroxy groups. Examples of functionalized resins which can serve as solid supports for immobilized solid phase synthesis are well-known in the art and include, without limitation, 4-methylbenzhydrylamine-copoly(styrene-1% divinylbenzene) (MBHA), 4-hydroxymethylphenoxymethyl-copoly(styrene-1% divinylbenzene), 4-oxymethyl-phenyl-acetamido-copoly(stryene-1% divinylbenzene) (Wang), 4-(oxymethyl)-phenylacetamido methyl (Pam), and Tentagel™, from Rapp Polymere Gmbh, trialkoxy-diphenyl-methyl ester-copoly(styrene-1% divinylbenzene)(RINK) all of which are commercially available. Other functionalized resins useful in the synthesis of the compounds of this invention will become apparent to those skilled in the art based on the disclosures herein. All such resins are within the scope of this invention.

When two substituents taken together along with the nitrogen or carbon atom to which they are attached form a ring, it is meant that the following structure:

—NR₁ₐR_{1b}

is a representative of the following structure:

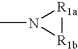

or that the following structure:

—CR₁ₐR_{1b}

is a representative of the following structure:

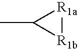

Thus, for example, “—NR₁ₐR_{1b}” can represent a heterocyclic substituent, such as pyridine, piperidine, morpholine, and the like, when R₁ₐ and R_{1b} taken together along with the nitrogen or carbon atom to which they are attached form a ring.

Throughout the present disclosure, when a particular compound comprises a chiral center, the scope of the present disclosure also includes compositions comprising the racemic mixture of the two enantiomers, as well as compositions comprising each enantiomer individually substantially free of the other enantiomer. Thus, for example, contemplated herein is a composition comprising the S enantiomer substantially free of the R enantiomer, or a composition comprising the R enantiomer substantially free of the S enantiomer. By “substantially free” it is meant that the composition comprises less than 10%, or less than 8%, or less than 5%, or less than 3%, or less than 1% of the minor enantiomer. If the particular compound comprises more than one chiral center, the scope of the present disclosure also includes compositions comprising a mixture of the various diastereomers, as well as compositions comprising each diastereomer substantially free of the other diastereomers. The recitation of a compound, without reference to any of its particular diastereomers, includes compositions comprising all four diastereomers, compositions comprising the racemic mixture of R,R and S,S isomers, compositions comprising the racemic mixture of R,S and S,R isomers, compositions comprising the R,R enantiomer substantially free of the other diastereomers, compositions comprising the S,S enantiomer substantially free of the other diastereomers, compositions comprising the R,S enantiomer substantially free of the other diastereomers, and compositions comprising the S,R enantiomer substantially free of the other diastereomers.

In another aspect, disclosed herein is a compound of Formula III:

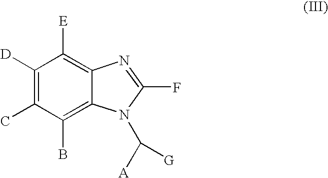

or a pharmaceutically acceptable salt or prodrug thereof, wherein:
A is selected from the group consisting of optionally substituted alkyl, optionally substituted alkenyl, optionally substituted alkynyl, optionally substituted cycloalkyl, optionally substituted (cycloalkyl)alkyl, optionally substituted cycloalkenyl, optionally substituted (cycloalkenyl)alkyl, optionally substituted aryl, optionally substituted heteroaryl, optionally substituted heteroalicyclyl, halogen, sulfenyl, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —C(R1)NC(=Z)NR₁ₐR_{1b}, —N(R₁)C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₂, —SR₂, and —OC(=Z)R₁,
B, C and E are each independently selected from the group consisting of hydrogen, optionally substituted alkyl, optionally substituted alkenyl, optionally substituted alkynyl, optionally substituted cycloalkyl, optionally substituted (cycloalkyl)alkyl, optionally substituted cycloalkenyl, optionally substituted (cycloalkenyl)alkyl, optionally substituted aryl, optionally substituted heteroaryl, optionally substituted heteroalicyclyl, halogen, nitro, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;
D is selected from the group consisting of optionally substituted heteroaryl, —C(=Z)R₁, —C(=Z)OR₁, —C(=Z)NR₁ₐR_{1b}, —C(=Z)N(R₁)NR₁ₐR_{1b}, —C(R₁)═NR₁ₐ, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, and —OC(=Z)R₁;
F is selected from the group consisting of optionally substituted alkyl, optionally substituted alkenyl, optionally substituted alkynyl, optionally substituted cycloalkyl, optionally substituted (cycloalkyl)alkyl, optionally substituted cycloalkenyl, optionally substituted (cycloalkenyl)alkyl, optionally substituted aryl, optionally substituted heteroaryl, optionally substituted aryloxy, optionally substituted heteroaryloxy; optionally substituted heteroalicyclyl, halogen, sulfenyl, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₂, —SR₂, and —OC(=Z)R₁;

G is selected from the group consisting of hydrogen, optionally substituted heteroaryl, —C(=Z)R₁, —C(=Z)OR₁, —C(=Z)NR₁ₐR_{1b}, —C(=Z)N(R₁)NR₁ₐR_{1b}, —C(R1)C(=Z)NR₁ₐR_{1b}, —C(R₁)═NR₁ₐ, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, and —OC(=Z)R₁;
Z is selected from the group consisting of oxygen, sulfur, and NR₁;
R₁, R₁ₐ, and R_{1b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl; or R₁ₐ, and R_{1b}, taken together with the nitrogen atom or carbon atom to which the are attached, form a five- or six-membered optionally substituted cycloalkyl, optionally substituted heterocycloalkyl, optionally substituted aryl, or optionally substituted heteroaryl ring; and
R₂ is selected from the group consisting of optionally substituted alkyl, optionally substituted alkenyl, optionally substituted alkynyl, optionally substituted cycloalkyl, optionally substituted (cycloalkyl)alkyl, optionally substituted cycloalkenyl, optionally substituted (cycloalkenyl)alkyl, optionally substituted aryl, optionally substituted heteroaryl, optionally substituted heteroalicyclyl;
wherein the alkyl, alkenyl, alkynyl, cycloalkyl, cycloalkenyl, aryl, heteroaryl, and heteroalicyclyl substituent of each of A, B, C, D, E, F, G. R₁, R₁ₐ, R_{1b}, and R₂ is each independently optionally substituted with one or more substituents selected from the group consisting of halogen, R₁, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —C(R₁)NC(=Z)NR₁ₐR_{1b}, —N(R₁)C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₂, —SR₂, and —OC(=Z)R₁.

In some embodiments, A is selected from the group consisting of optionally substituted C₁-C₈ alkyl, optionally substituted C₁-C₈ alkenyl, optionally substituted C₁-C₈ alkynyl, optionally substituted C₃-C₈ cycloalkyl, optionally substituted C₃-C₈ cycloalkenyl, optionally substituted aryl, optionally substituted heteroaryl, optionally substituted heteroalicyclyl, halogen, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁, wherein the alkyl, alkenyl, alkynyl, cycloalkyl, cycloalkenyl, aryl, heteroaryl, and heteroalicyclyl is each independently optionally substituted with one or more substituents selected from the group consisting of halogen, alkoxy, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁.

In certain embodiments, A is selected from the group consisting of optionally substituted C₁-C₈ alkyl, optionally substituted C₃-C₈ cycloalkyl, optionally substituted aryl, optionally substituted heteroaryl, and —N(R₁)—C(=Z)NR₁ₐR_{1b}, wherein the alkyl, cycloalkyl, aryl, and heteroaryl is each independently optionally substituted with one or more substituents selected from the group consisting of halogen, alkoxy, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁.

In other embodiments, A is selected from the group consisting of C₁-C₈ alkyl optionally substituted with one or more —N(R₁)C(═NR₁)NR₁ₐR_{1b}. In some of these embodiments, R₁, R₁ₐ, R_{1b} and are hydrogen.

In some of the above embodiments, the alkyl is selected from the group consisting of methyl, ethyl, n-propyl, iso-propyl, n-butyl, sec-butyl, iso-butyl, and tert-butyl.

In some embodiments, B, C and E are each independently selected from the group consisting of hydrogen, optionally substituted C₁-C₈ alkyl, optionally substituted C₁-C₈ alkenyl, optionally substituted C₁-C₈ alkynyl, optionally substituted C₃-C₈ cycloalkyl, optionally substituted C₃-C₈ cycloalkenyl, optionally substituted aryl, optionally substituted heteroaryl, optionally substituted heteroalicyclyl, halogen, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁, wherein the alkyl, alkenyl, alkynyl, cycloalkyl, cycloalkenyl, aryl, heteroaryl, and heteroalicyclyl is each independently optionally substituted with one or more substituents selected from the group consisting of halogen, alkoxy, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁.

In other embodiments, B, C and E are each independently selected from the group consisting of hydrogen, optionally substituted C₁-C₈ alkyl, optionally substituted C₃-C₈ cycloalkyl, optionally substituted aryl, optionally substituted heteroaryl, and —N(R₁)—C(=Z)NR₁ₐR_{1b}, wherein the alkyl, cycloalkyl, aryl, and heteroaryl is each independently optionally substituted with one or more substituents selected from the group consisting of halogen, alkoxy, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁.

In some embodiments, B is hydrogen; while in other embodiments, C is hydrogen; and in still other embodiments, E is hydrogen.

In some embodiments, D is selected from the group consisting of —C(=Z)R₁, —C(=Z)OR₁, —C(=Z)NR₁ₐR_{1b}, —C(=Z)N(R₁)NR₁ₐR_{1b}, —C(R₁)═NR₁ₐ, —N(R₁)—C(=Z)R₁, —N(R₁)—C(=Z)NR₁ₐR_{1b}, and —OC(=Z)R₁. In some of these embodiments, D is —C(═O)NR₁ₐR_{1b}.

In some embodiments, when referring to the substituent D, R₁ₐ is selected from the group consisting of hydrogen, optionally substituted C₁-C₈ alkyl, optionally substituted C₂-C₈ alkenyl, optionally substituted C₂-C₈ alkynyl, optionally substituted amine, optionally substituted C₃-C₈ cycloalkyl, optionally substituted C₃-C₈ cycloalkenyl, optionally substituted aryl, optionally substituted ar-C₁-C₈-alkyl, optionally substituted heteroaryl, optionally substituted heteroaryl-C₁-C₈-alkyl, and optionally substituted heteroalicyclyl.

In other embodiments, when referring to the substituent D, R₁ₐ is hydrogen or optionally substituted C₁-C₈ alkyl. In some of these embodiments, the alkyl is selected from the group consisting of methyl, ethyl, n-propyl, iso-propyl, n-butyl, sec-butyl, iso-butyl, and tert-butyl.

In certain embodiments, when referring to the substituent D, R_{1b} is selected from the group consisting of hydrogen, optionally substituted C₁-C₈ alkyl, optionally substituted C₂-C₈ alkenyl, optionally substituted C₂-C₈ alkynyl, optionally substituted amine, optionally substituted C₃-C₈ cycloalkyl, optionally substituted C₃-C₈ cycloalkenyl, optionally substituted aryl, optionally substituted ar-C₁-C₈-alkyl, optionally substituted heteroaryl, optionally substituted heteroaryl-C₁-C₈-alkyl, and optionally substituted heteroalicyclyl.

In some embodiments, when referring to the substituent D, R_{1b} is optionally substituted C₁-C₈ alkyl. In some of these embodiments, the alkyl is selected from the group consisting of methyl, ethyl, n-propyl, iso-propyl, n-butyl, sec-butyl, iso-butyl, and tert-butyl. In further embodiments, the alkyl is optionally substituted with one or more substituents selected from the group consisting of optionally substituted aryl, optionally substituted heteroaryl, and optionally substituted heteroalicyclyl. In yet other embodiments, the alkyl is optionally substituted with pyridine, pyrrolidine, pyrrolidinone, phenyl, methoxyphenyl, fluorophenyl, trifluoromethylphenyl, indazole, N-morpholine, and piperazine.

In some embodiments, when referring to the substituent D, R_{1b} is optionally substituted aryl. In some embodiments, the aryl is phenyl. In certain embodiments, the aryl, e.g., phenyl, is optionally substituted with one or more substituents selected from the group consisting of amino, methylamino, ethylamino, dimethylamino, diethylamino, methyl, ethyl, n-propyl, iso-propyl, n-butyl, sec-butyl, iso-butyl, tert-butyl, methoxy, ethoxy, fluoro, chloro, bromo, and trifluoromethyl.

In some embodiments, when referring to the substituent D, R_{1b} is optionally substituted heteroaryl or optionally substituted heteroalicyclyl, which can be selected from the group consisting indazole, thiazole, and isothiazole.

In some embodiments, when referring to the substituent D, R₁ₐ and R_{1b}, taken together with the nitrogen atom to which they are attached, form a five- or six-membered optionally substituted cycloalkyl, optionally substituted heterocycloalkyl, optionally substituted aryl, or optionally substituted heteroaryl ring. In some of these embodiments, R₁ₐ and R_{1b}, taken together with the nitrogen atom to which they are attached, form an optionally substituted morpholine or an optionally substituted piperazine. In further embodiments, the morpholine or piperazine is independently optionally substituted with one or more substituents selected from the group consisting of methyl, phenyl, fluorophenyl, trifluoromethylphenyl, methylphenyl, methoxyphenyl, and methoxy.

In some embodiments, F is selected from the group consisting of optionally substituted C₁-C₈ alkyl, optionally substituted C₂-C₈ alkenyl, optionally substituted C₂-C₈ alkynyl, optionally substituted C₃-C₈ cycloalkyl, optionally substituted C₃-C₈ cycloalkenyl, optionally substituted aryl, optionally substituted heteroaryl, optionally substituted aryloxy, optionally substituted heteroaryloxy; optionally substituted heteroalicyclyl, wherein the alkyl, alkenyl, alkynyl, cycloalkyl, cycloalkenyl, aryl, heteroaryl, and heteroalicyclyl substituent is each independently optionally substituted with one or more substituents selected from the group consisting of halogen, R₁, nitro, —CN, —NR₁ₐR_{1b}, —N(R₁)—C(=Z)R₁, —OR₂, —SR₂, and —OC(=Z)R₁.

In other embodiments, F is selected from the group consisting of optionally substituted aryl and optionally substituted heteroaryl, wherein the aryl and heteroaryl is each independently optionally substituted with one or more substituents selected from the group consisting of halogen, R₁, nitro, —CN, —NR₁ₐR_{1b}, —N(R₁)—C(═O)R₁, and —OR₂. In some of these embodiments, the aryl is phenyl, which can be optionally substituted with one or more substituents selected from the group consisting of chloro, bromo, methyl, ethyl, n-propyl, iso-propyl, n-butyl, sec-butyl, iso-butyl, tert-butyl, phenyl, nitro, —CN, —NH₂, —N(CH₃)₂, —NHC(═O)CH₃, —N(CH₃)C(═O)CH₃, hydroxy, methoxy, trifluoromethoxy, phenoxy, benzyloxy, and phenylmercaptyl, where the phenoxy or phenylmercaptyl is each optionally substituted with one or more substituents selected from the group consisting of chloro, trifluoromethyl, methyl, ethyl, n-propyl, iso-propyl, n-butyl, sec-butyl, iso-butyl, tert-butyl, and methoxy.

In some embodiments, A is —CH₂CH₂CH₂NHC(═NH)NH₂.

In some embodiments, B is hydrogen.

In some embodiments, C is hydrogen.

In some embodiments, D is selected from the group consisting of:

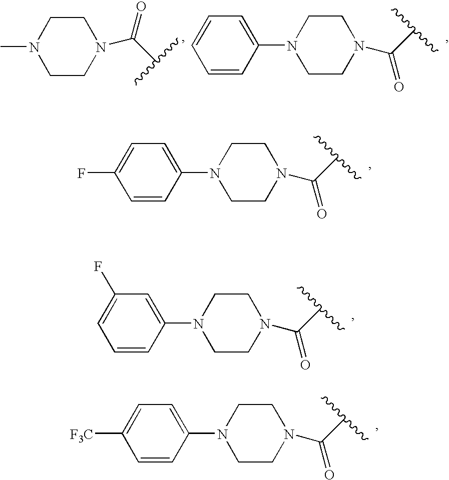

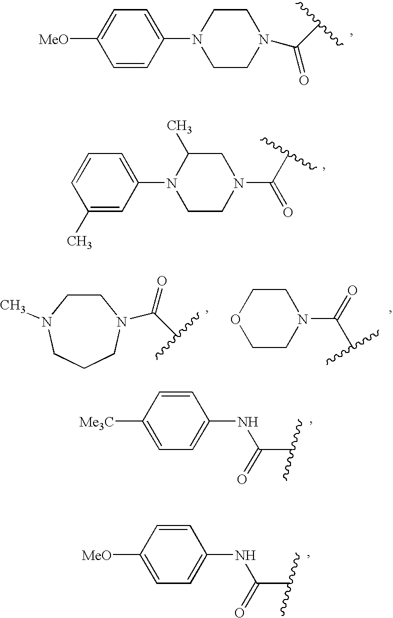

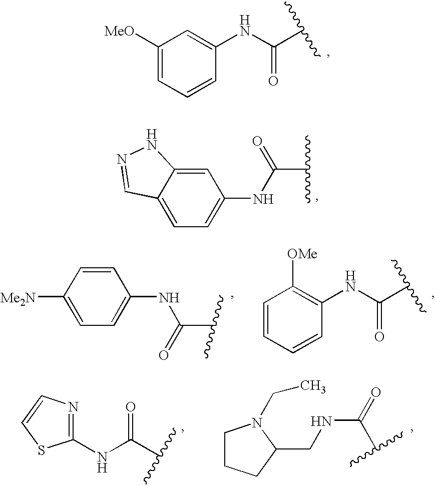

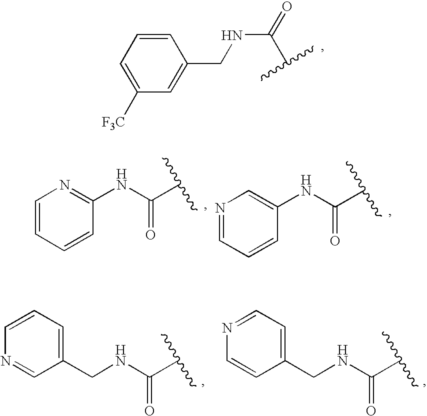

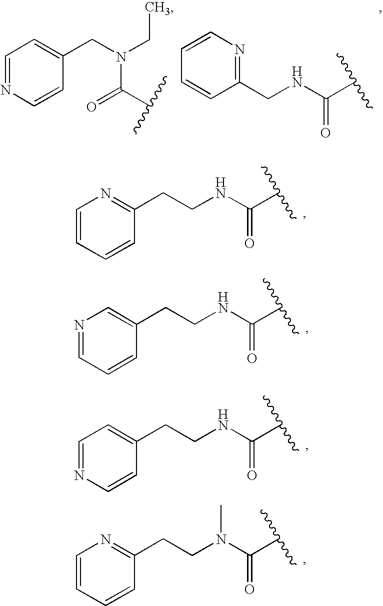

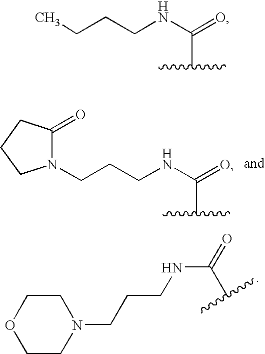

In some embodiments, E is hydrogen.

In some embodiments, F is selected from the group consisting of:

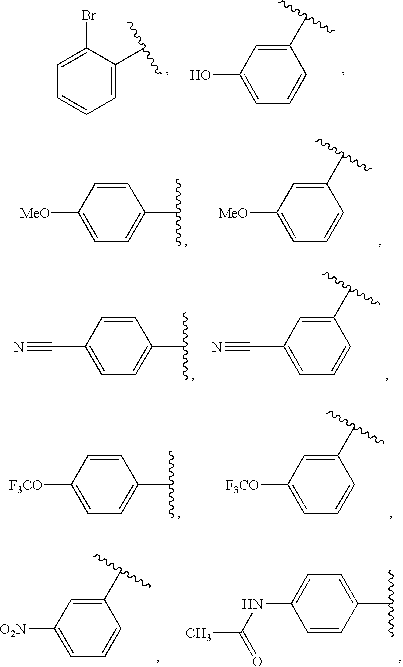

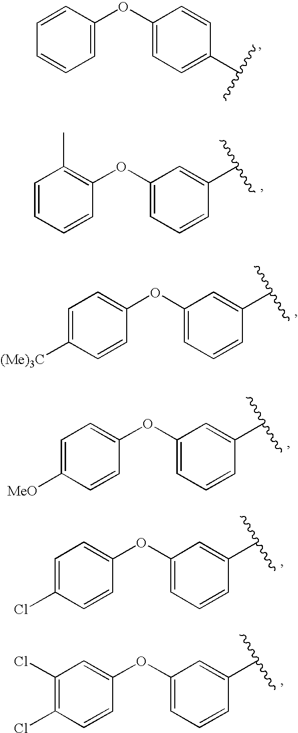

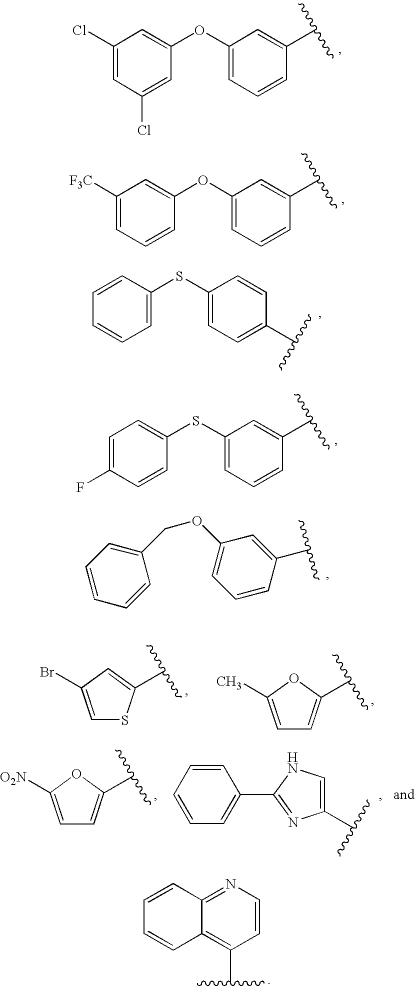

In some embodiments, G is —C(═O)NH₂.

An embodiment of the invention is a compound of Formula III, wherein:

A is selected from the group consisting of C₁-C₈ alkyl, C₁-C₈alkenyl, C₁-C₈ alkynyl, cycloalkyl, cycloalkenyl, substituted or unsubstituted aryl, substituted or unsubstituted heteroaryl, substituted or unsubstituted heteroalicyclyl, sulfenyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₃, —SR₃, and —OC(=Z)R₁; —C(R₁)NC(═N)NR₁ₐR_{1b},

B, C and E are independently selected from the group consisting of hydrogen, alkyl, alkenyl, alkynyl, cycloalkyl, cycloalkenyl, substituted or unsubstituted aryl, substituted or unsubstituted heteroaryl, substituted or unsubstituted heteroalicyclyl, halogen, nitro, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;

D is selected from the group consisting of —C(═O)R₁, —C(═O)NR₁ₐR_{1b}, —C(═O)N(R₁)NR₁ₐR_{1b}, —C(R₁)═NR₁ₐ, —N(R₁)—C(═O)R₁, —N(R₁)—C(═O)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, and —OC(═O)R₁;

F is selected from the group consisting of substituted or unsubstituted aryl, substituted or unsubstituted heteroaryl, substituted or unsubstituted aryloxy, substituted or unsubstituted heteroaryloxy, —C(=Z)R₁, —C(=Z)OR₁, —C(=Z)NR₁ₐR_{1b}, —C(=Z)N(R₁)NR₁ₐR_{1b}, —C(R₁)═NR₁ₐ, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, and —OC(=Z)R₁;

G is absent or selected from the group consisting of —C(=Z)R₁, —C(=Z)OR₁, —C(=Z)NR₁ₐR_{1b}, —C(=Z)N(R₁)NR₁ₐR_{1b}, —C(R₁)═NR₁ₐ, —N(R₁)—C(=Z)R₁, —C(R1)NC(═N)NR₁ₐR_{1b}, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;

Z is oxygen;

R₁, R₁ₐ and R_{1b} are independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heteroalicyclyl; and

R₃ is selected from the group consisting of substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heteroalicyclyl.

In another embodiment, disclosed herein is a compound of Formula III, wherein:

A is selected from the group consisting of C₁-C₈ alkyl, C₁-C₈ alkenyl, C₁-C₈alkynyl, cycloalkyl, cycloalkenyl, substituted or unsubstituted aryl, substituted or unsubstituted heteroaryl, substituted or unsubstituted heteroalicyclyl, sulfenyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₃, —SR₃, and —OC(=Z)R₁;

B, C and E are independently selected from the group consisting of hydrogen, alkyl, alkenyl, alkynyl, cycloalkyl, cycloalkenyl, substituted or unsubstituted aryl, substituted or unsubstituted heteroaryl, substituted or unsubstituted heteroalicyclyl, halogen, nitro, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;

D is selected from the group consisting of —C(═O)R₁, —C(═O)NR₁ₐR_{1b}, —C(═O)N(R₁)NR₁ₐR_{1b}, —C(R₁)═NR₁ₐ, —N(R₁)—C(═O)R₁, —N(R₁)—C(═O)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, and —OC(═O)R₁;

F is selected from the group consisting of substituted or unsubstituted aryl, substituted or unsubstituted heteroaryl;

G is selected from the group consisting of —C(=Z)R₁, —C(=Z)OR₁, —C(=Z)NR₁ₐR_{1b}, —C(=Z)N(R₁)NR₁ₐR_{1b}, —S(O)NR₁ₐR_{1b}, —S(O)₂NR₁ₐR_{1b}, —OR₁, —SR₁, and —OC(=Z)R₁;

Z is oxygen;

R₁, R₁ₐ and R_{1b} are independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heteroalicyclyl; and

R₃ is selected from the group consisting of substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heteroalicyclyl.

In yet another embodiment, D is selected from the group consisting of, —S(O)R₄, —S(O)₂R₄, —C(═O)R₄, and —C(═O)N(R₁)R₄; wherein:

R₄ is selected from the group consisting of:

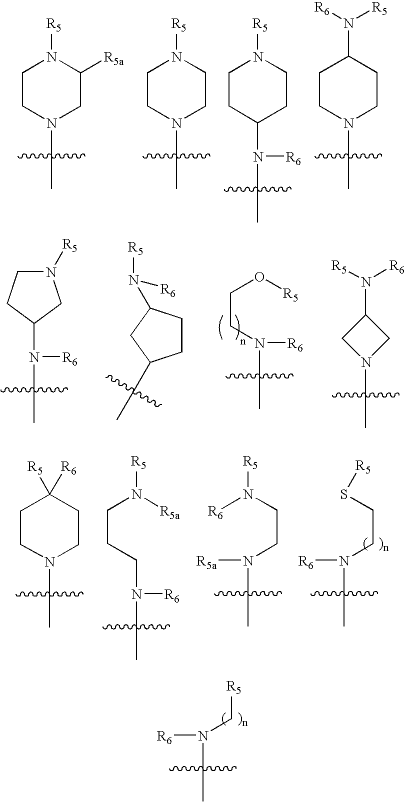

wherein:

n is an integer selected from the group consisting of 1, 2, 3, 4, 5, 6 or 7 defining the number of methylene groups; and

R₅, R₅ₐ and R₆ are independently selected from the group consisting of hydrogen, alkyl, alkenyl, alkynyl, cycloalkyl, cycloalkenyl, substituted or unsubstituted aryl, substituted or unsubstituted heteroaryl, substituted or unsubstituted heteroalicyclyl, —C(═O)R₁, —C(═O)OR₁, —C(═O)NR₁ₐR_{1b}, —NR₁ₐR_{1b}, —N═CR₁ₐR_{1b}, —N(R₁)—C(═O)R₁, —S(O)₂R₁ₐ, —S(═O)R₁, —S(═O)₂R₁, and —OR₁.

In another embodiment, R₅ is a substituted or unsubstituted aryl or substituted or unsubstituted heteroaryl either of which may be substituted with zero to five substituents each independently selected from the group consisting of alkyl, alkenyl, alkynyl, cycloalkyl, cycloalkenyl, substituted or unsubstituted aryl, substituted or unsubstituted heteroaryl, substituted or unsubstituted heteroalicyclyl, halogen, nitro, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;

In another embodiment, F is a substituted or unsubstituted aryl or substituted or unsubstituted heteroaryl either of which may be substituted with zero to five substituents each independently selected from the group consisting of alkyl, alkenyl, alkynyl, cycloalkyl, cycloalkenyl, substituted or unsubstituted aryl, substituted or unsubstituted heteroaryl, substituted or unsubstituted heteroalicyclyl, halogen, nitro, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;

In another embodiment,

A is selected from the group consisting of hydrogen, methyl, 2-propyl, 2-butyl, aminocarbonylethyl, 2-methylmercaptoethyl, phenyl, benzyl, cyclohexylmethyl, 4-methoxybenzyl, 4-chlorobenzyl, 3-indolylmethyl, 4-(trifluoroacetyl)aminobutyl and 3-guanidinopropyl;

G is selected from the group consisting of —C(=Z)OR₁, —C(=Z)NR₁ₐR_{1b}, —S(O)NR₁ₐR_{1b}, —S(O)₂NR₁ₐR_{1b};

Z is oxygen; and

R₁, R₁ₐ and R_{1b} are independently selected from the group consisting of hydrogen, substituted or unsubstituted alkyl;

In another aspect, disclosed herein is a compound selected from the group consisting of

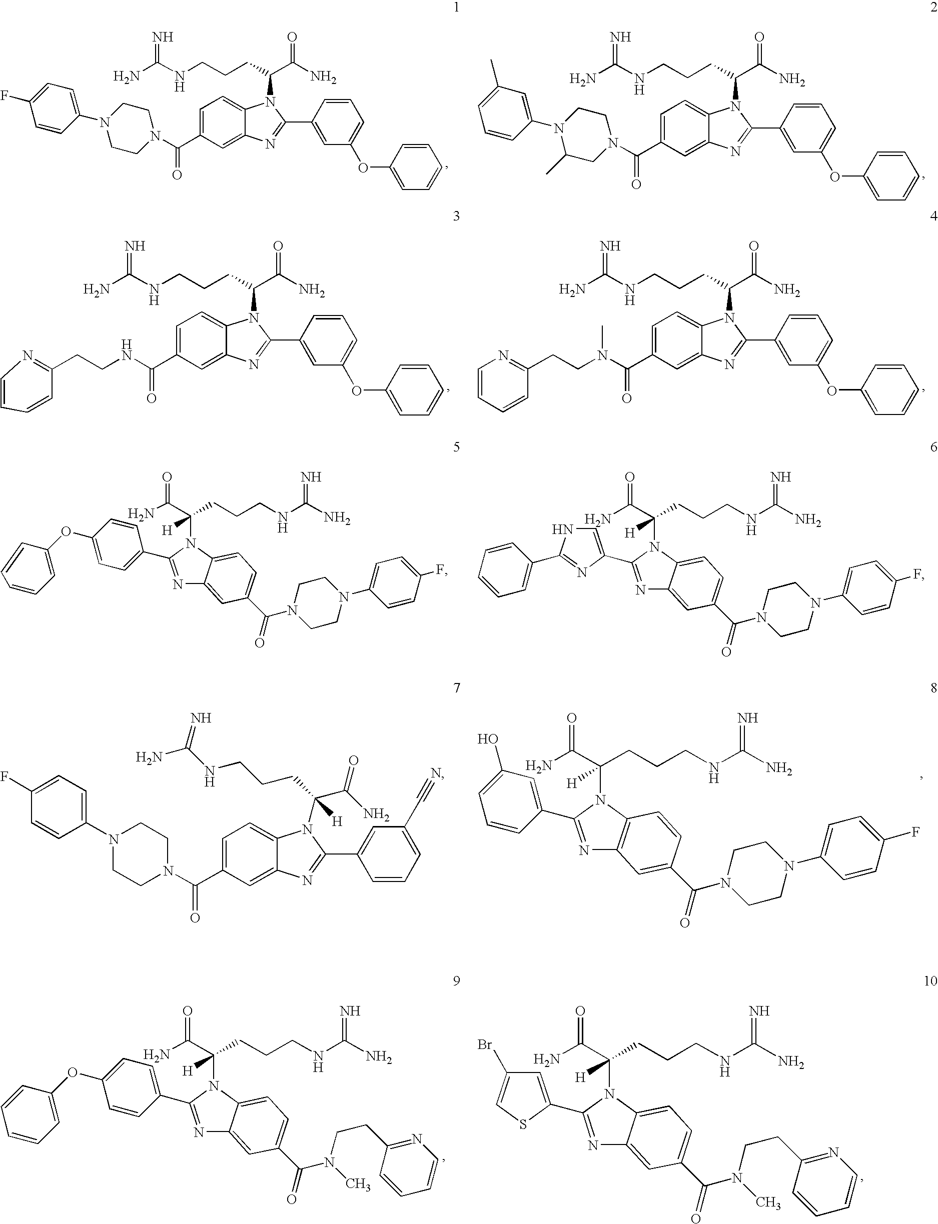

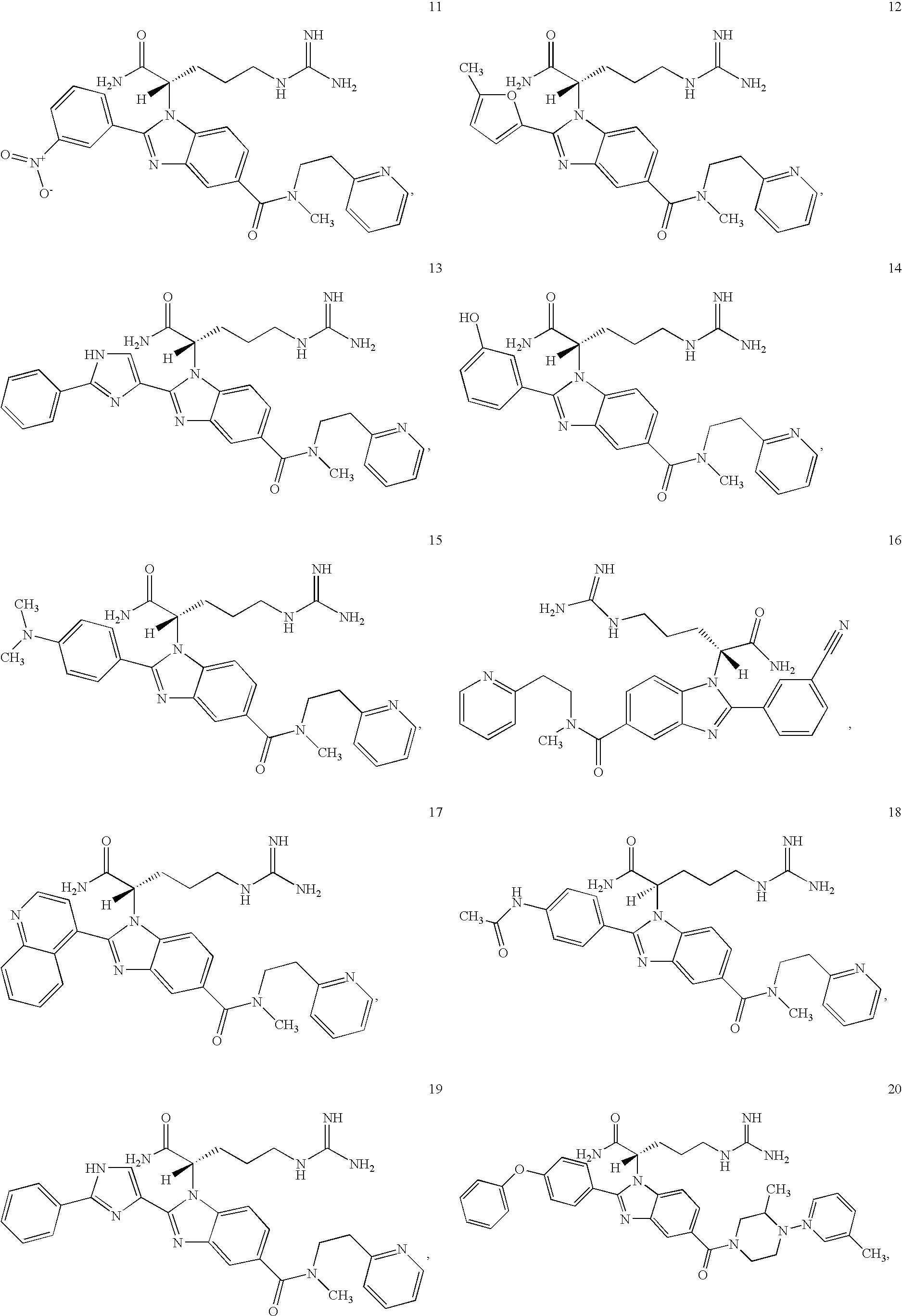

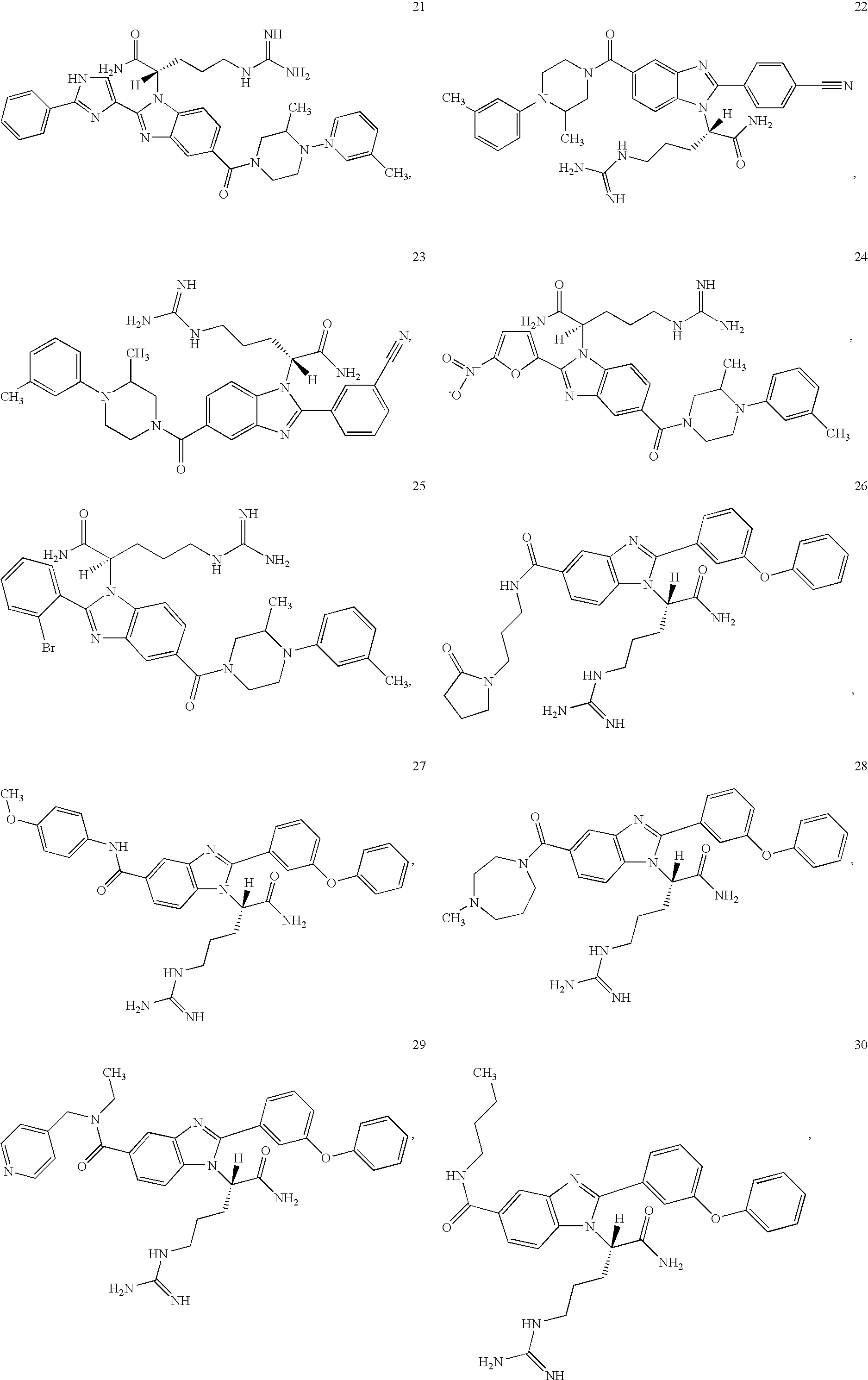

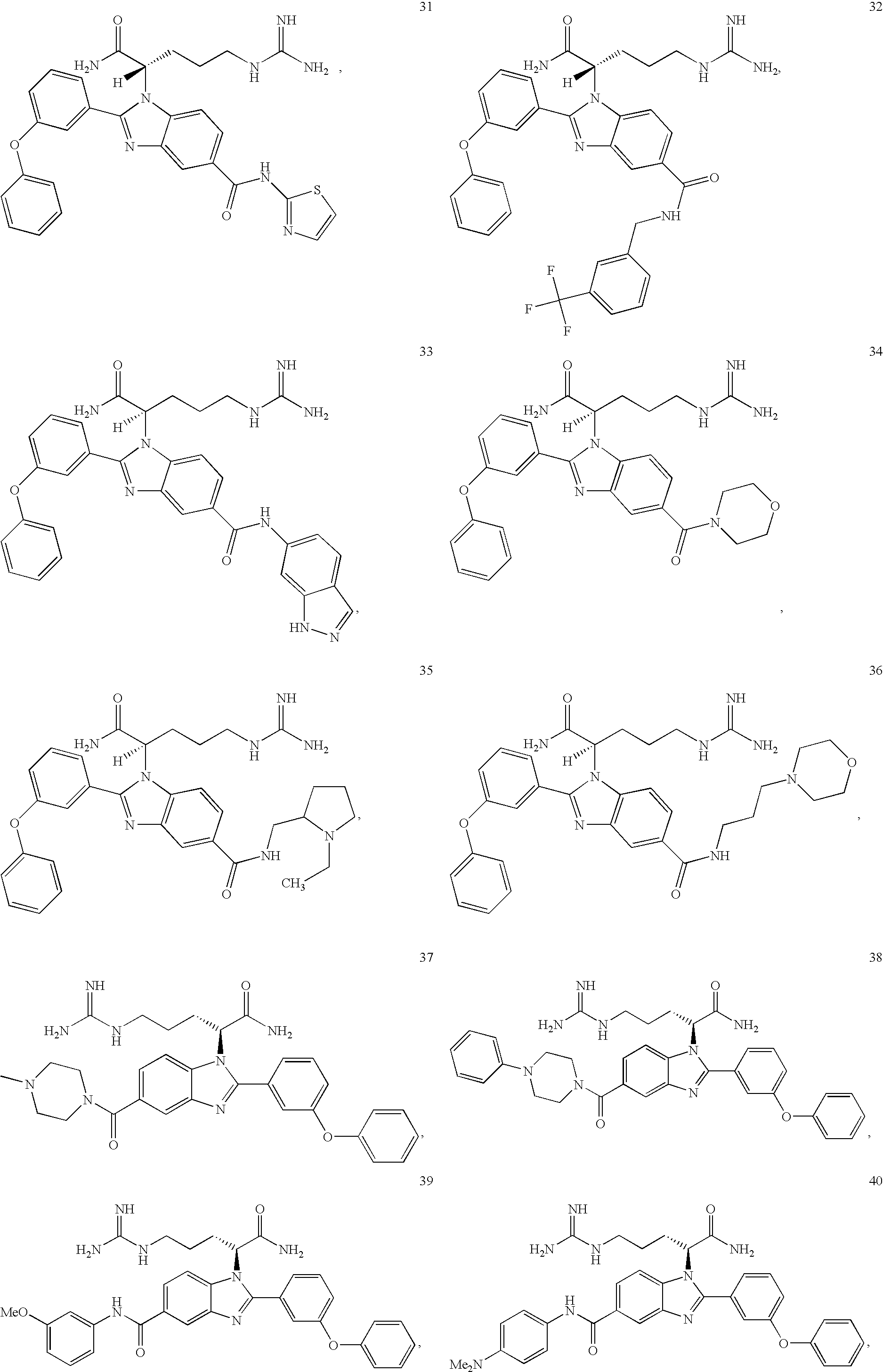

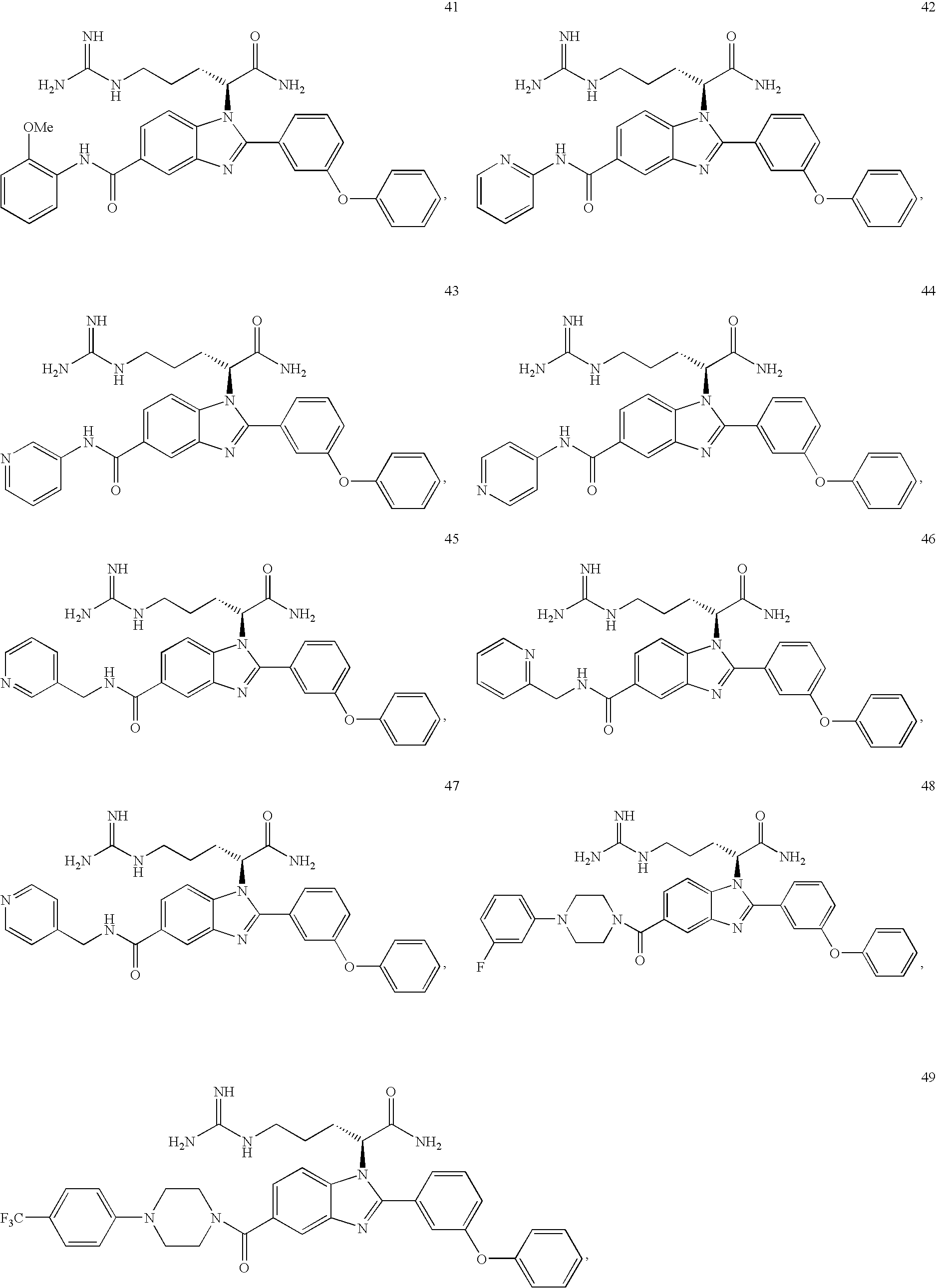

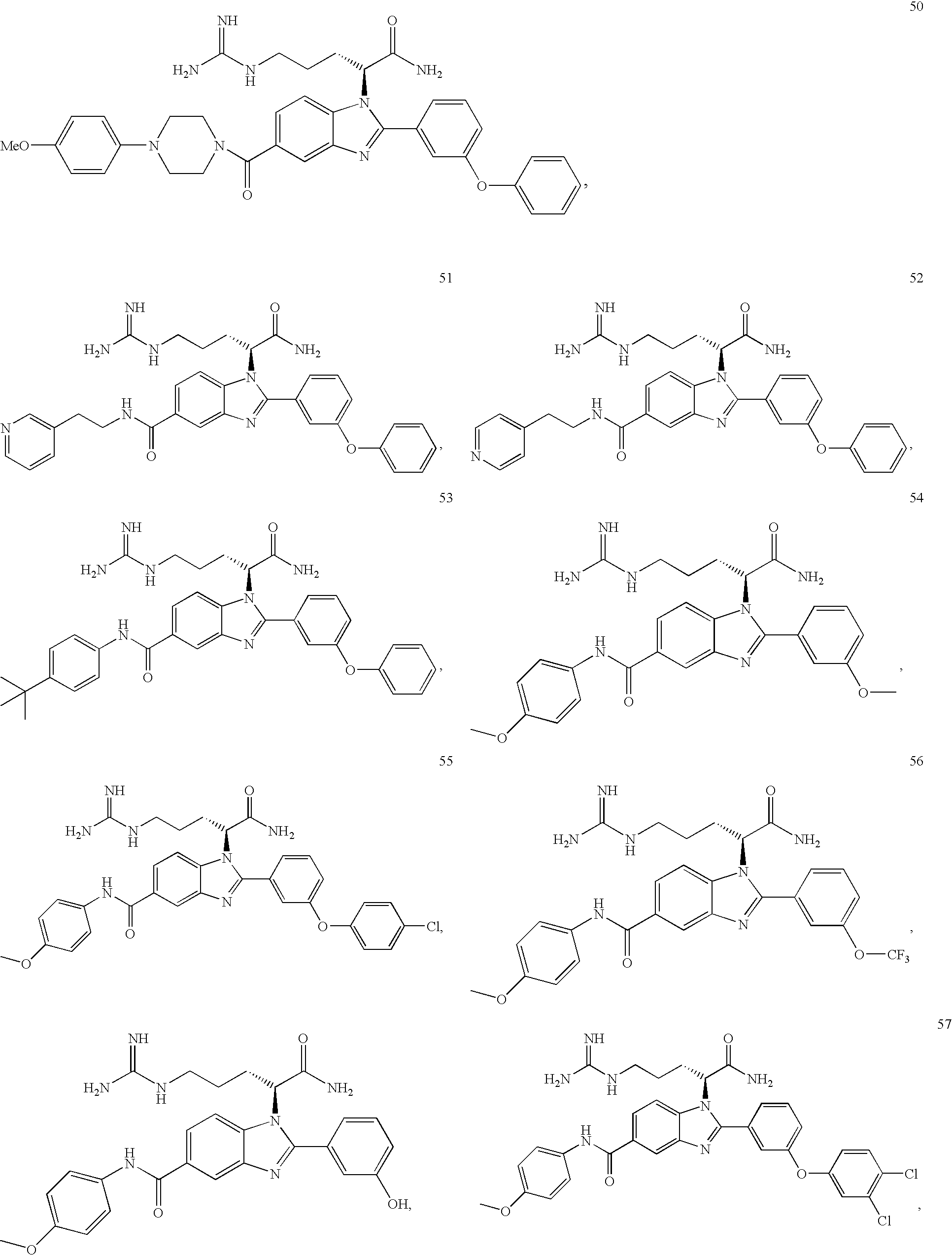

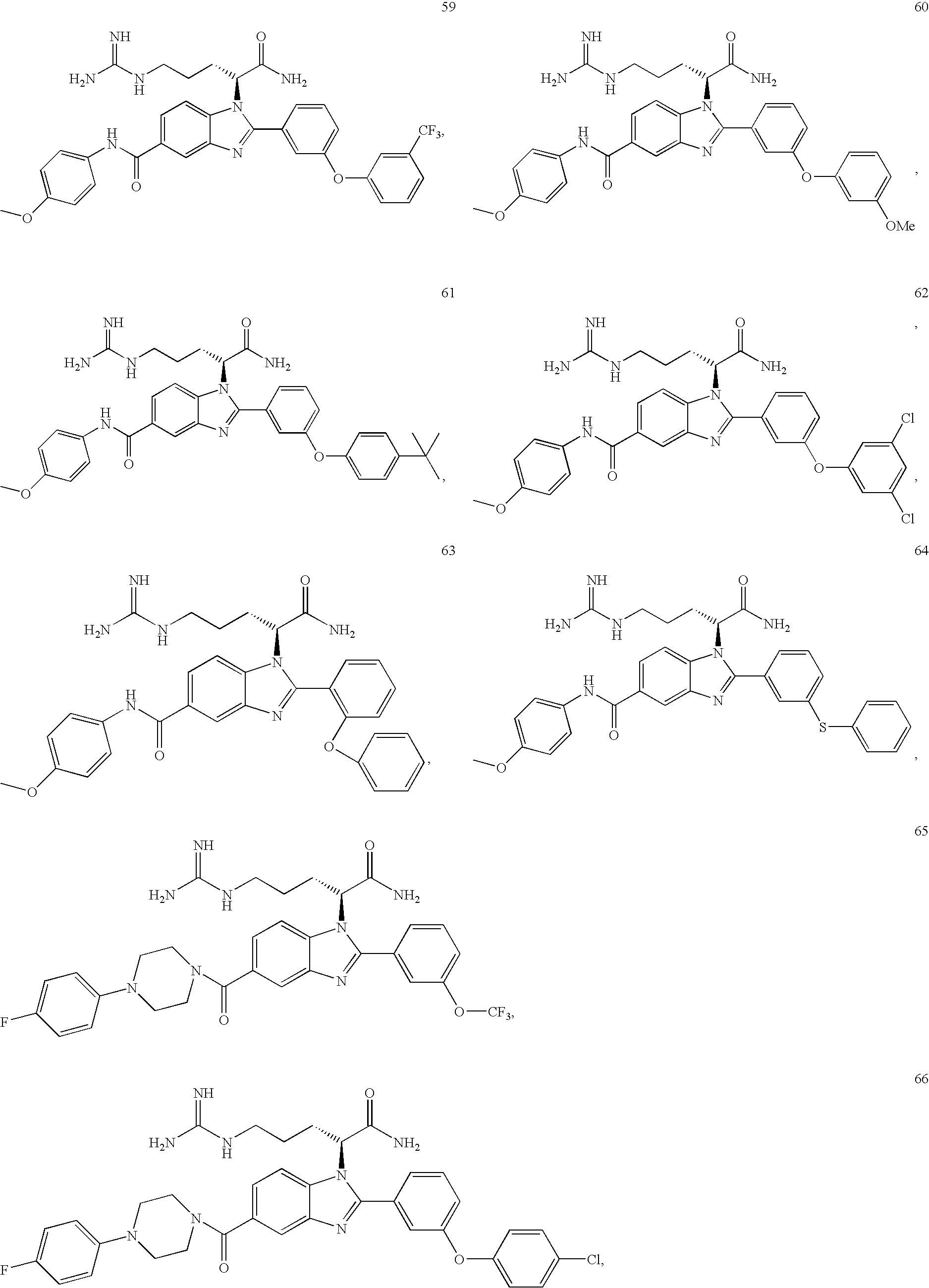

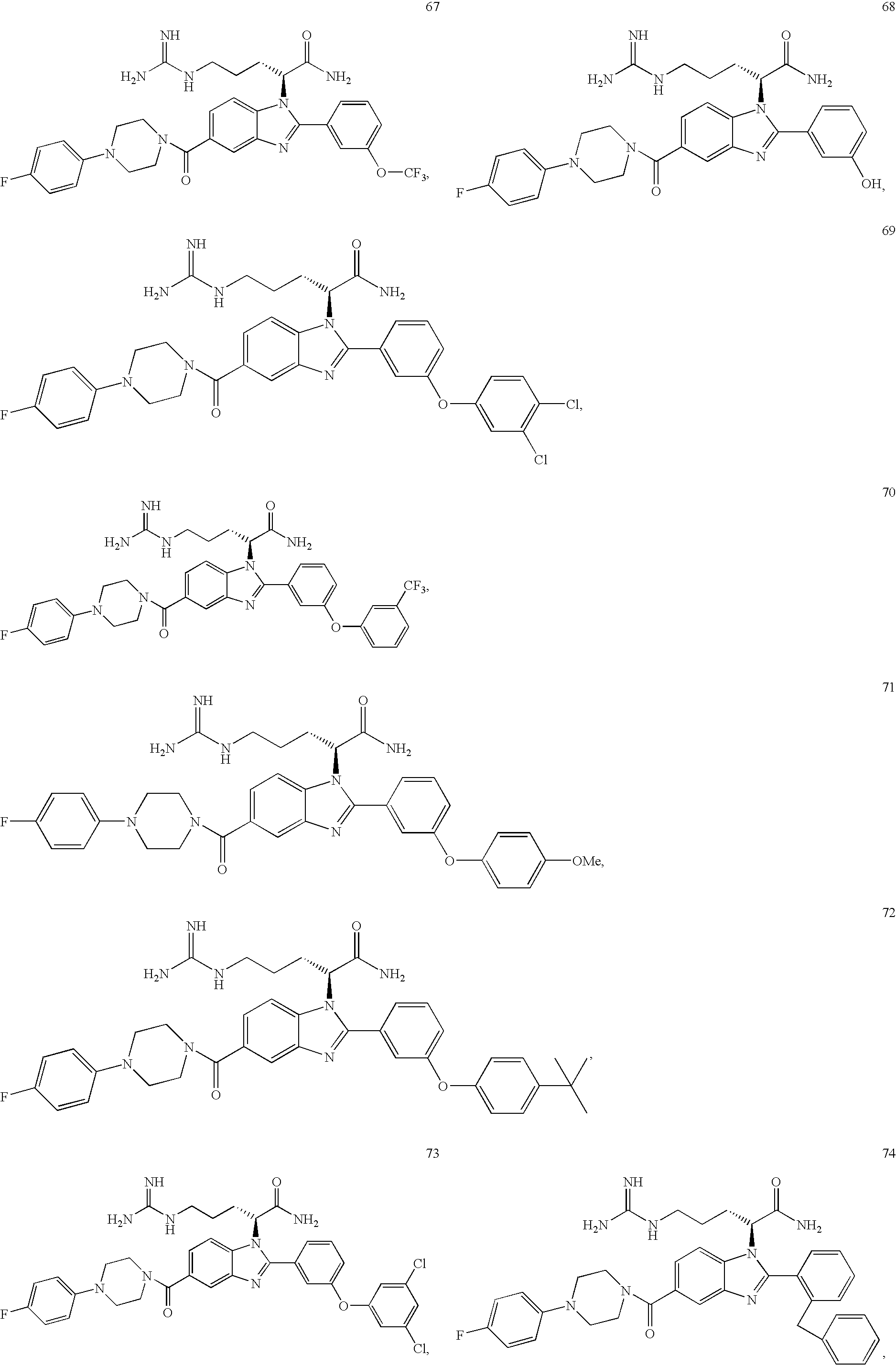

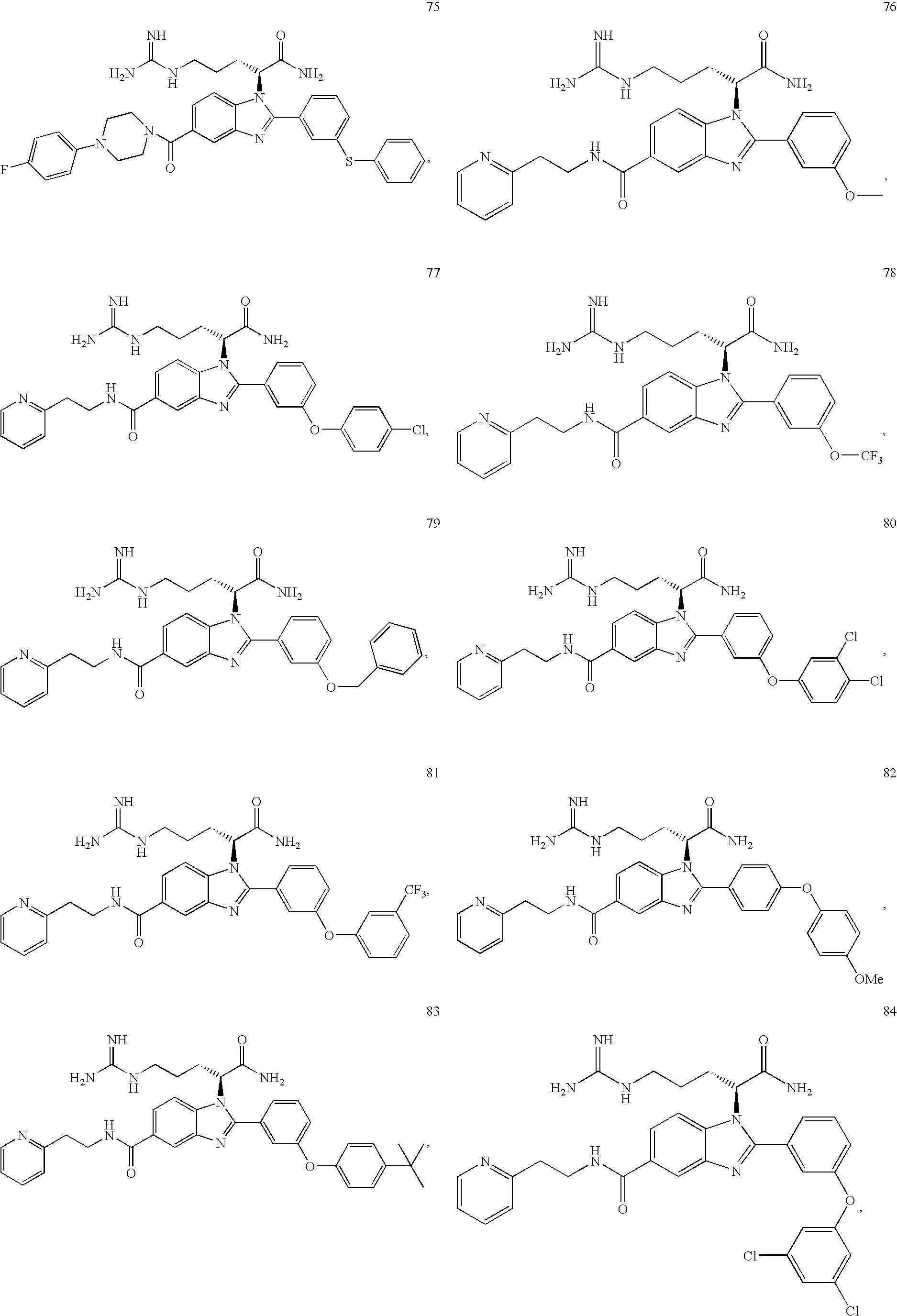

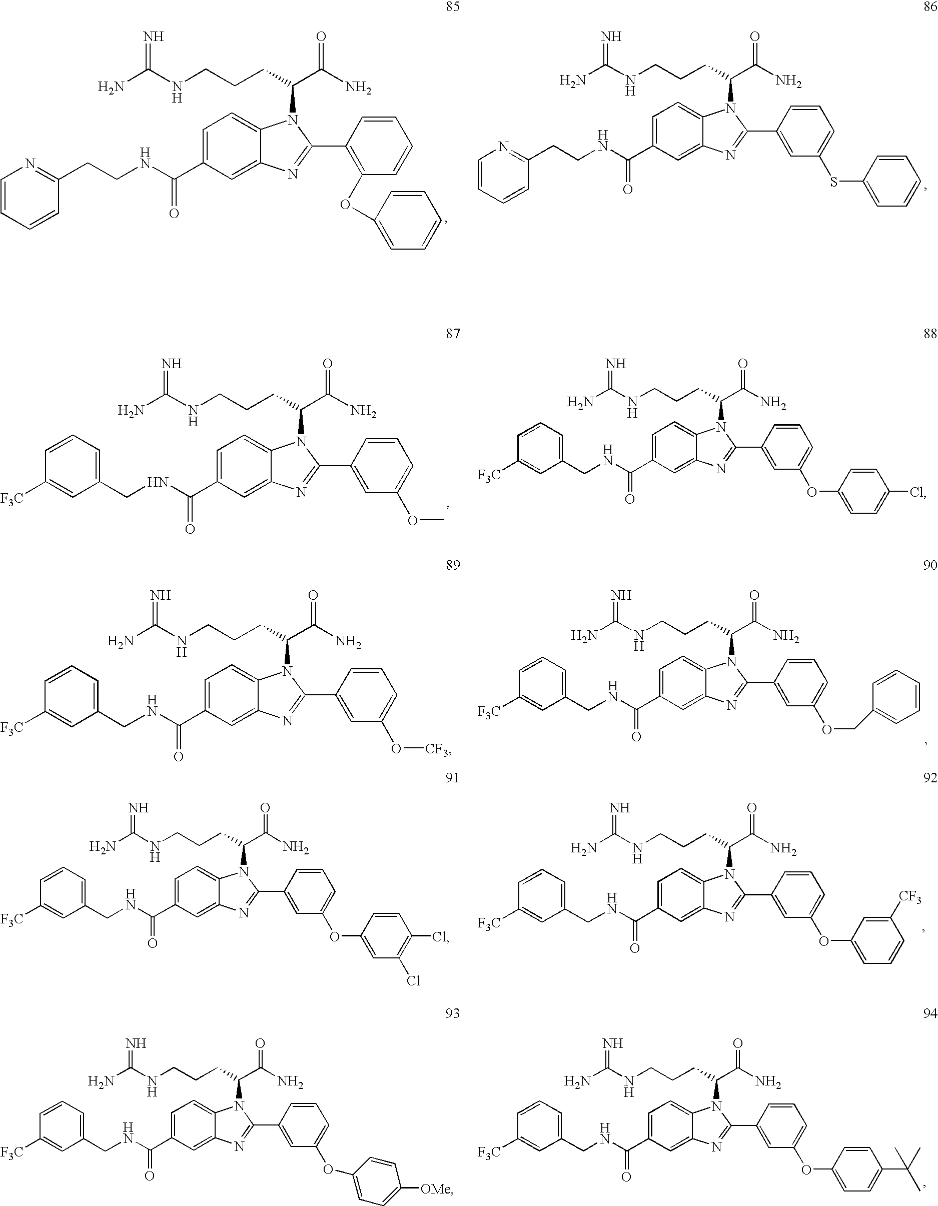

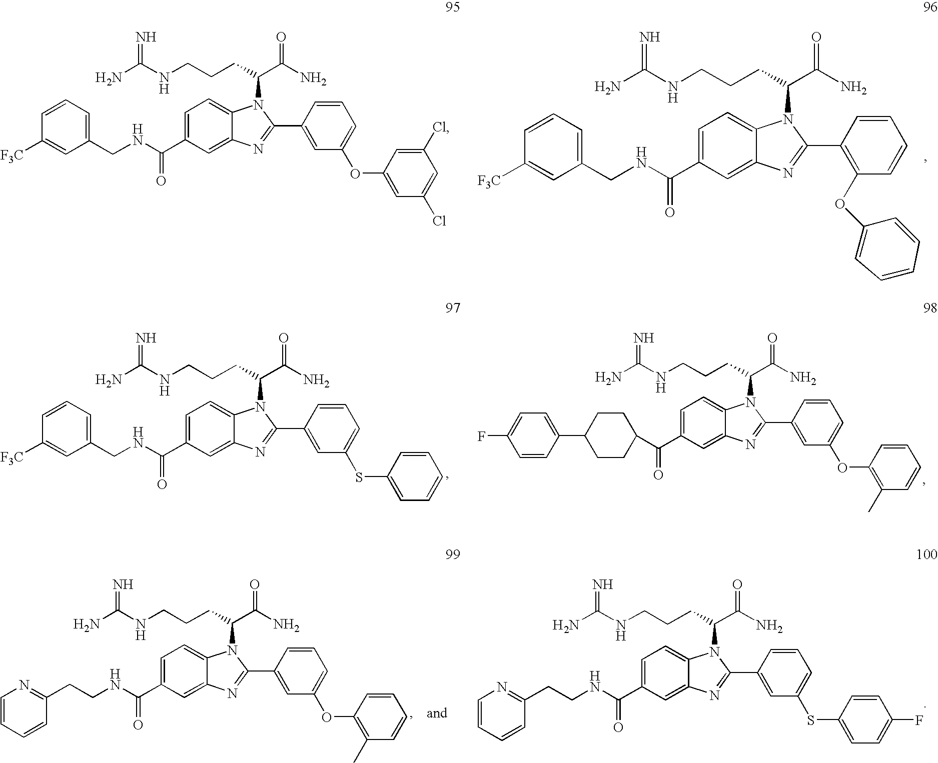

In another aspect, disclosed herein is a compound of Formula I:

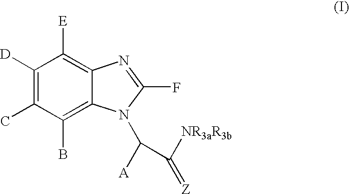

or a pharmaceutically acceptable salt or prodrug thereof, wherein:
A, B, C, E, F, and Z are each as described above;
D is selected from the group consisting of —C(=Z)NR₄ₐR_{4b}, —C(=Z)N(R₄)NR₄ₐR_{4b}, —C(R₄)═NR₄ₐ, —N(R₄)—C(=Z)R₄, and —N(R₄)—C(=Z)NR₄ₐR_{4b};
R₁, R₁ₐ, R_{1b} and R₂ are each as described above;
R₃ₐ and R_{3b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl; or R₃ₐ and R_{3b}, taken together with the nitrogen atom to which they are attached, form a five- or six-membered optionally substituted cycloalkyl, optionally substituted heterocycloalkyl, optionally substituted aryl, or optionally substituted heteroaryl ring; and
R₄, R₄ₐ, and R_{4b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl;
where the alkyl, alkenyl, alkynyl, cycloalkyl, cycloalkenyl, aryl, heteroaryl, and heteroalicyclyl substituent of each of A, B, C, D, E, F, R₁, R₁ₐ, R_{1b}, R₂, R₃ₐ, R_{3b}, R₄, R₄ₐ, and R_{4b} is each independently optionally substituted with one or more substituents selected from the group consisting of halogen, R₁, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —C(R₁)NC(=Z)NR₁ₐR_{1b}, —N(R₁)C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₂, —SR₂, and —OC(=Z)R₁.

In some embodiments, D is —C(═O)NR₄ₐR_{4b}.

In some embodiments, R₄ₐ and R_{4b} and is each independently selected from the group consisting of hydrogen, optionally substituted C₁-C₈ alkyl, optionally substituted C₂-C₈ alkenyl, optionally substituted C₂-C₈ alkynyl, optionally substituted amine, optionally substituted C₃-C₈ cycloalkyl, optionally substituted C₃-C₈ cycloalkenyl, optionally substituted aryl, optionally substituted ar-C₁-C₈-alkyl, optionally substituted heteroaryl, optionally substituted heteroaryl-C₁-C₈-alkyl, and optionally substituted heteroalicyclyl.

In other embodiments, R₄ₐ and R_{4b} is each independently hydrogen or optionally substituted C₁-C₈ alkyl.

In some embodiments, R₃ₐ and R_{3b} is each independently hydrogen. In further embodiments, in the compound of Formula I:
A is selected from the group consisting of optionally substituted C₁-C₈ alkyl, optionally substituted C₃-C₈ cycloalkyl, optionally substituted aryl, optionally substituted heteroaryl, and —N(R₁)—C(=Z)NR₁ₐR_{1b}, wherein the alkyl, cycloalkyl, aryl, and heteroaryl is each independently optionally substituted with one or more substituents selected from the group consisting of halogen, alkoxy, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁,
B, C and E are each independently selected from the group consisting of hydrogen, optionally substituted C₁-C₈ alkyl, optionally substituted C₃-C₈ cycloalkyl, optionally substituted aryl, optionally substituted heteroaryl, and —N(R₁)—C(=Z)NR₁ₐR_{1b}, wherein the alkyl, cycloalkyl, aryl, and heteroaryl is each independently optionally substituted with one or more substituents selected from the group consisting of halogen, alkoxy, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁;
D is —C(=Z)NR₄ₐR_{4b};
F is selected from the group consisting of optionally substituted C₁-C₈ alkyl, optionally substituted C₂-C₈ alkenyl, optionally substituted C₂-C₈ alkynyl, optionally substituted C₃-C₈ cycloalkyl, optionally substituted C₃-C₈ cycloalkenyl, optionally substituted aryl, optionally substituted heteroaryl, optionally substituted aryloxy, optionally substituted heteroaryloxy; optionally substituted heteroalicyclyl, wherein the alkyl, alkenyl, alkynyl, cycloalkyl, cycloalkenyl, aryl, heteroaryl, and heteroalicyclyl substituent is each independently optionally substituted with one or more substituents selected from the group consisting of halogen, R₁, nitro, —CN, —NR₁ₐR_{1b}, —N(R₁)—C(=Z)R₁, —OR₂, —SR₂, and —OC(=Z)R₁;
Z is selected from the group consisting of oxygen, sulfur, and NR₁; and
R₃ₐ and R_{3b} are each independently hydrogen or optionally substituted alkyl.

In some embodiments, the compound of Formula I is selected from the group consisting of

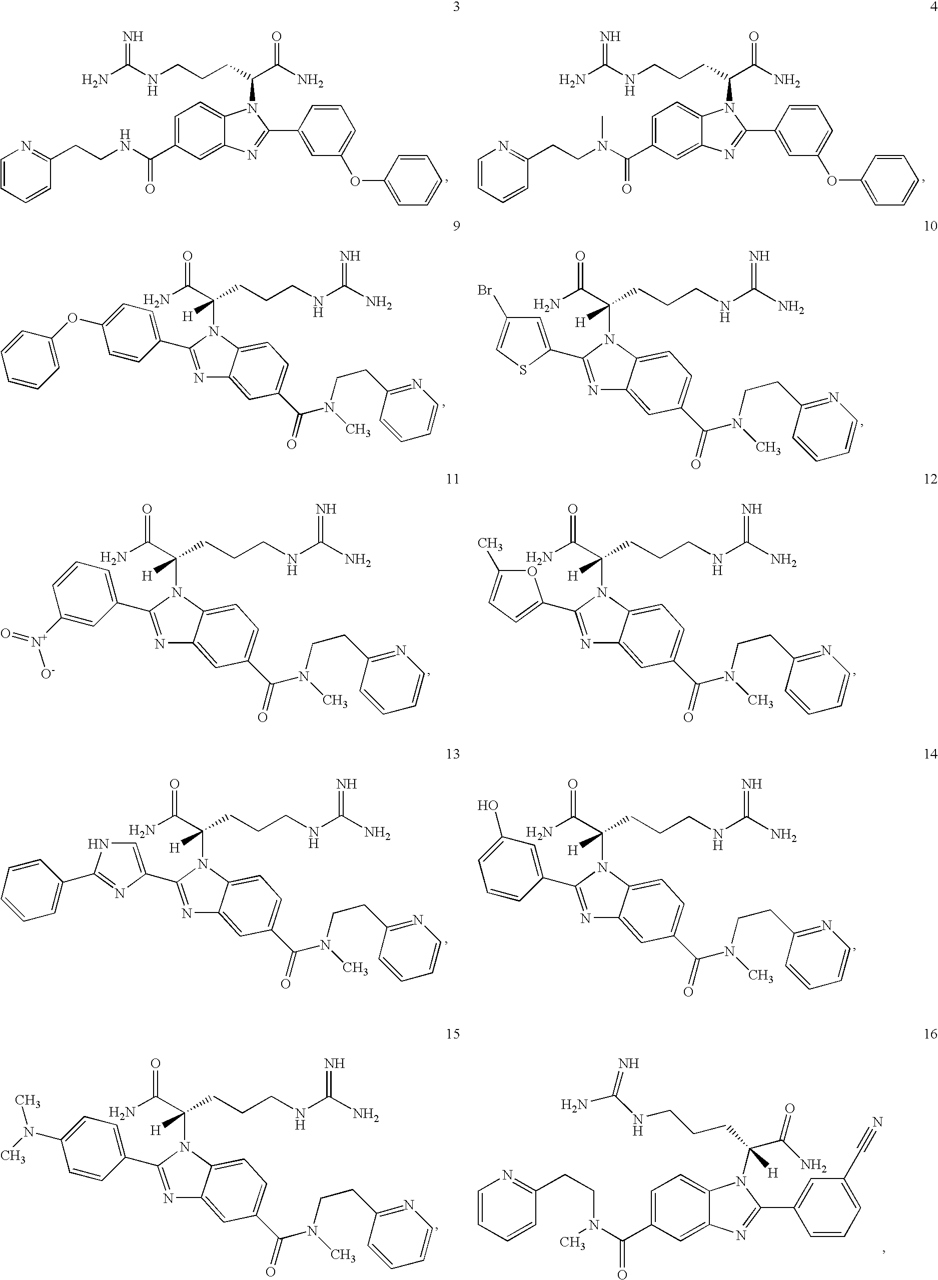

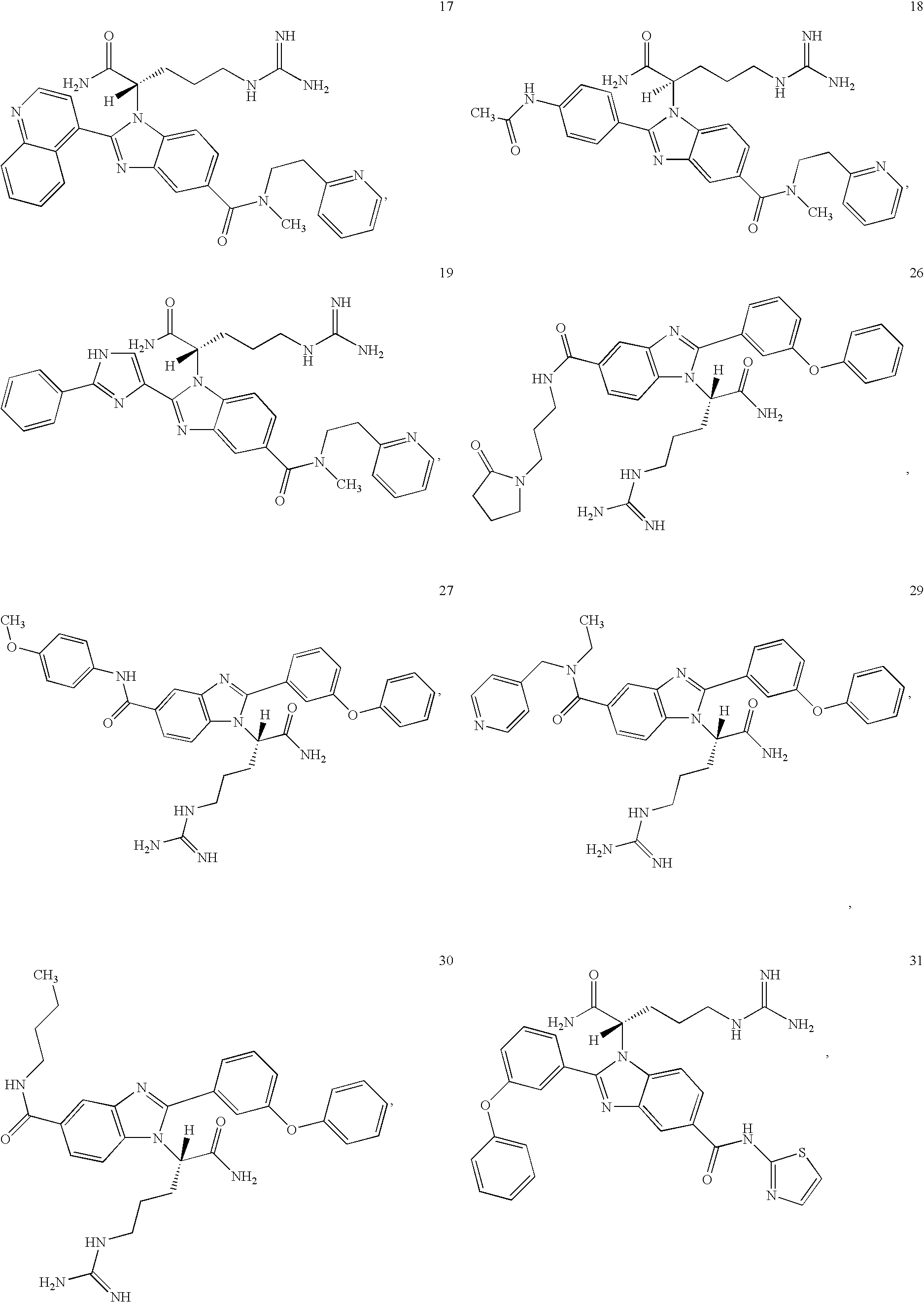

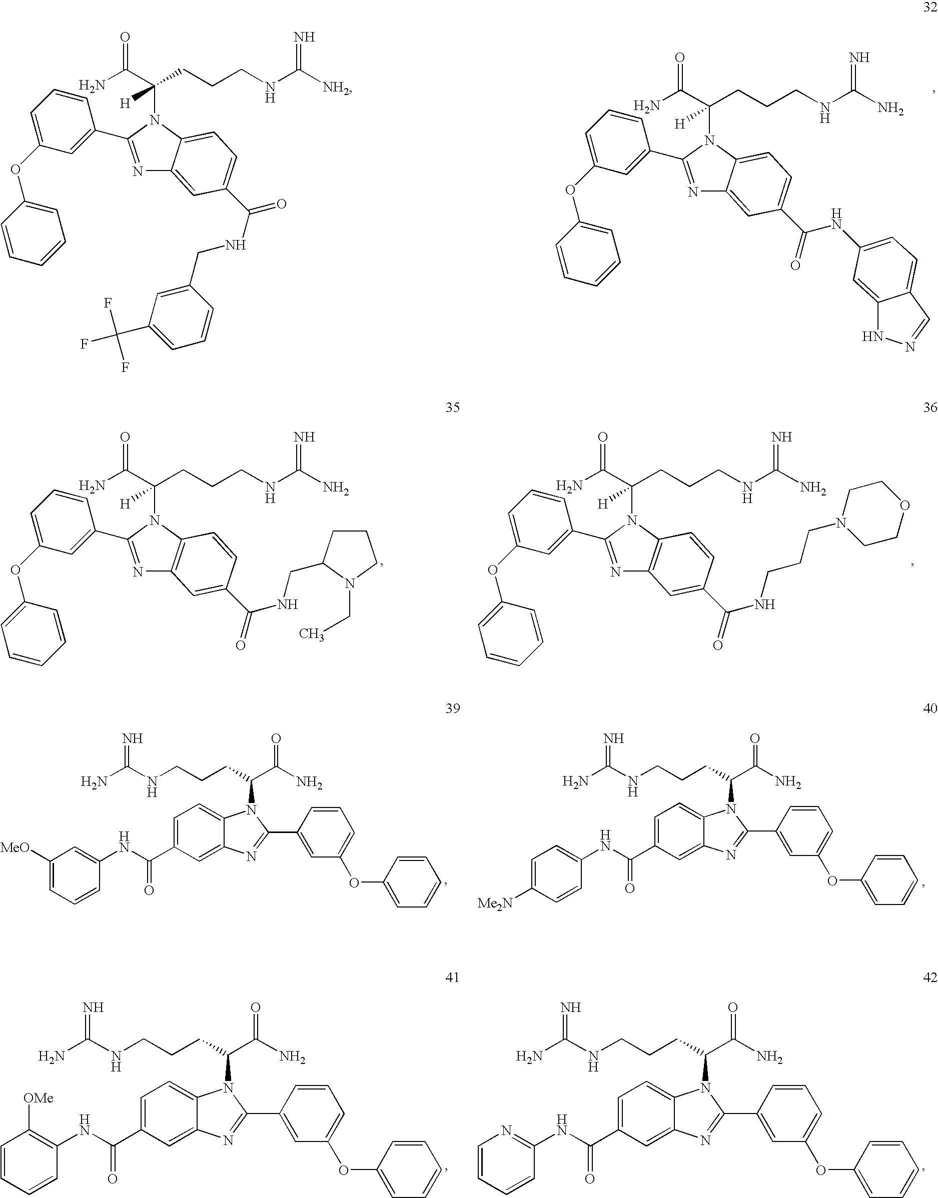

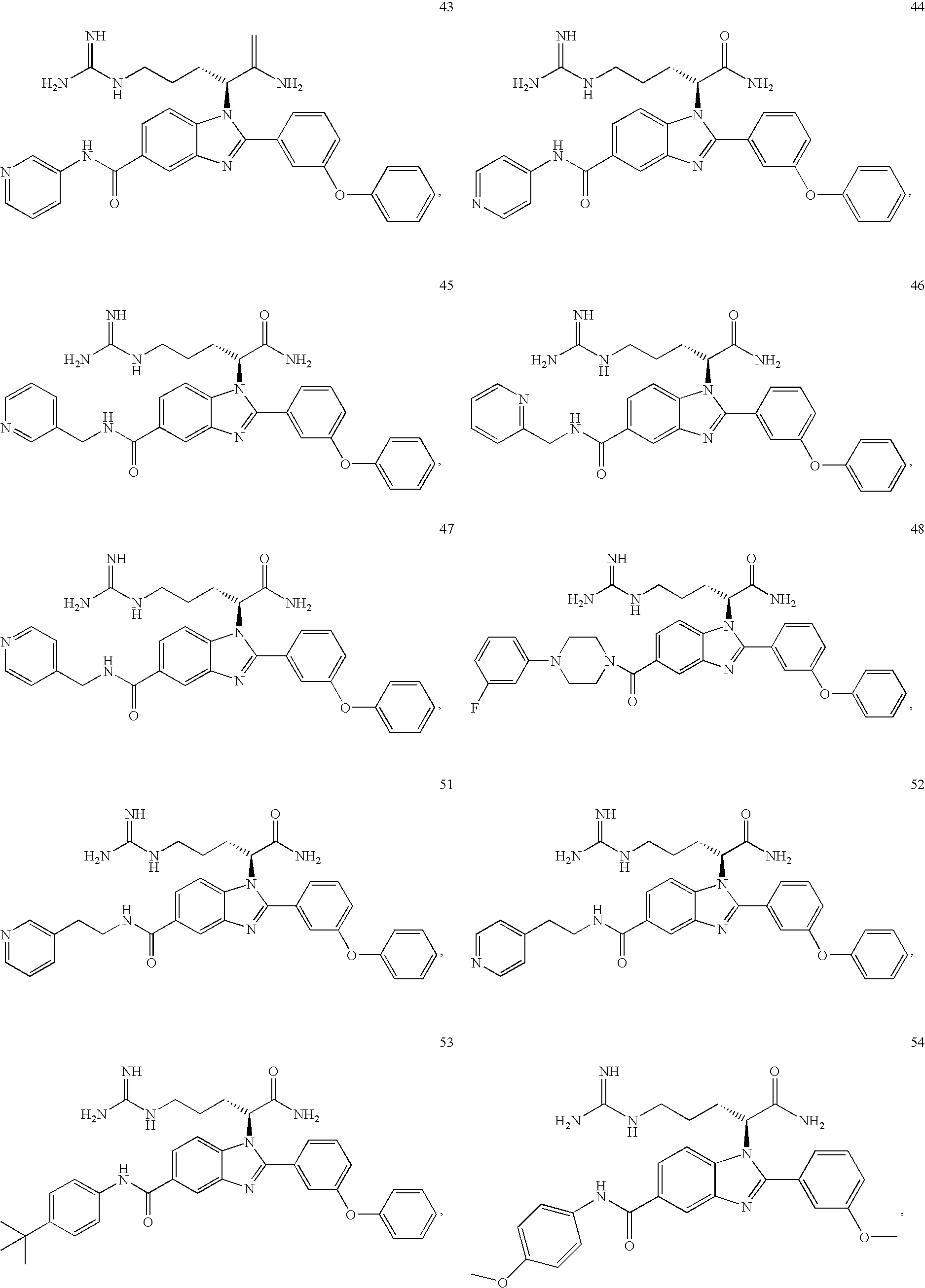

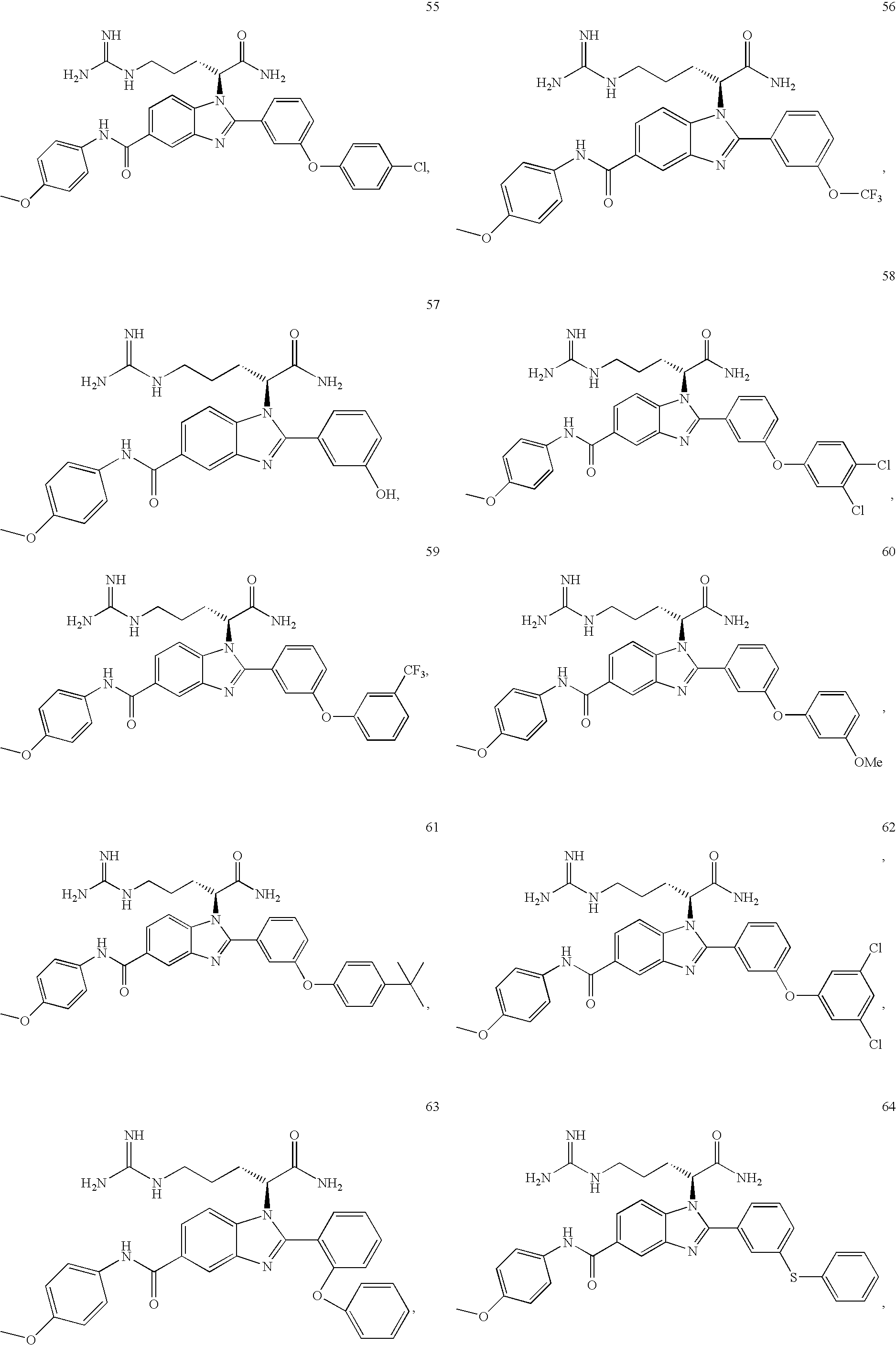

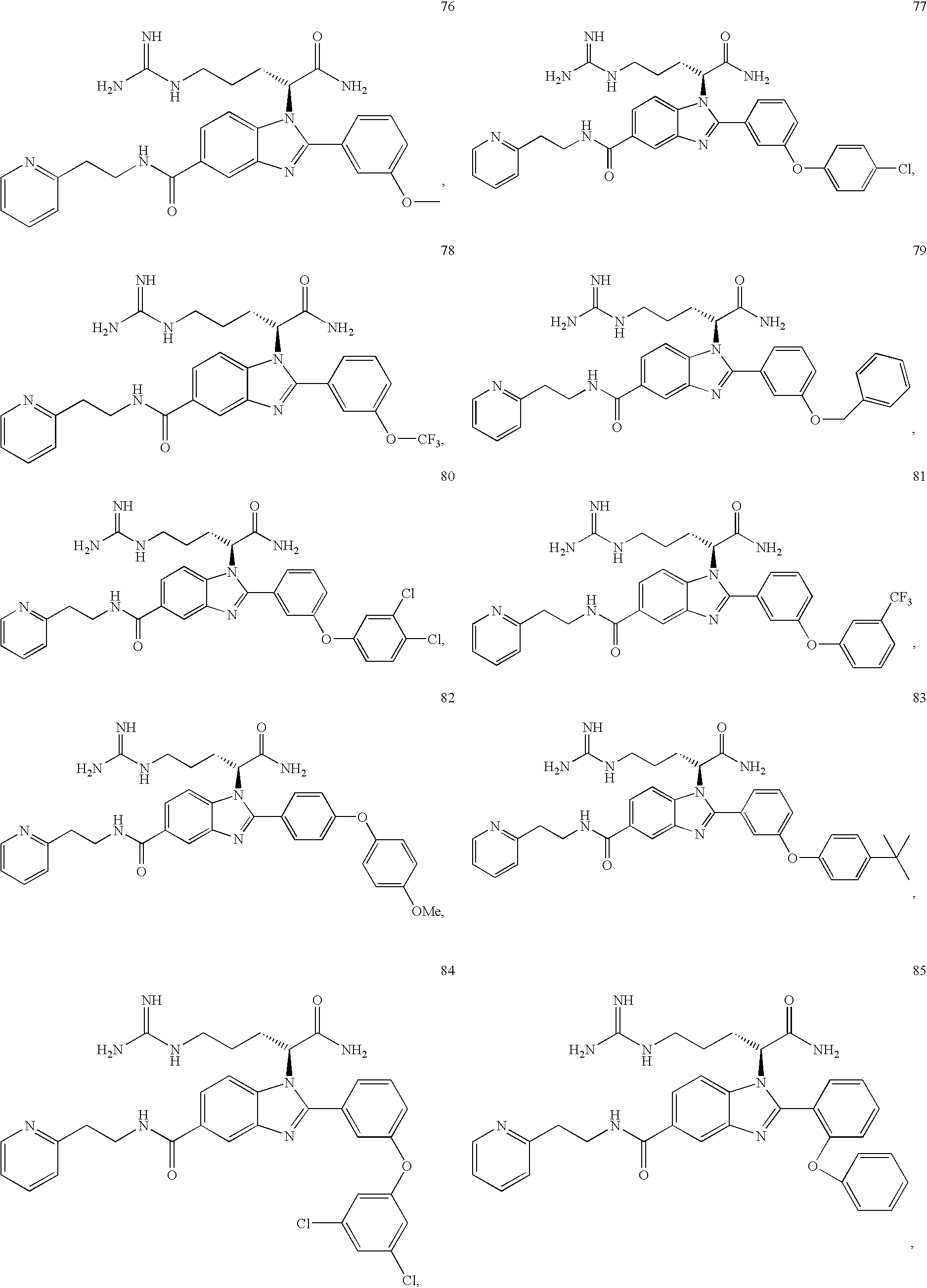

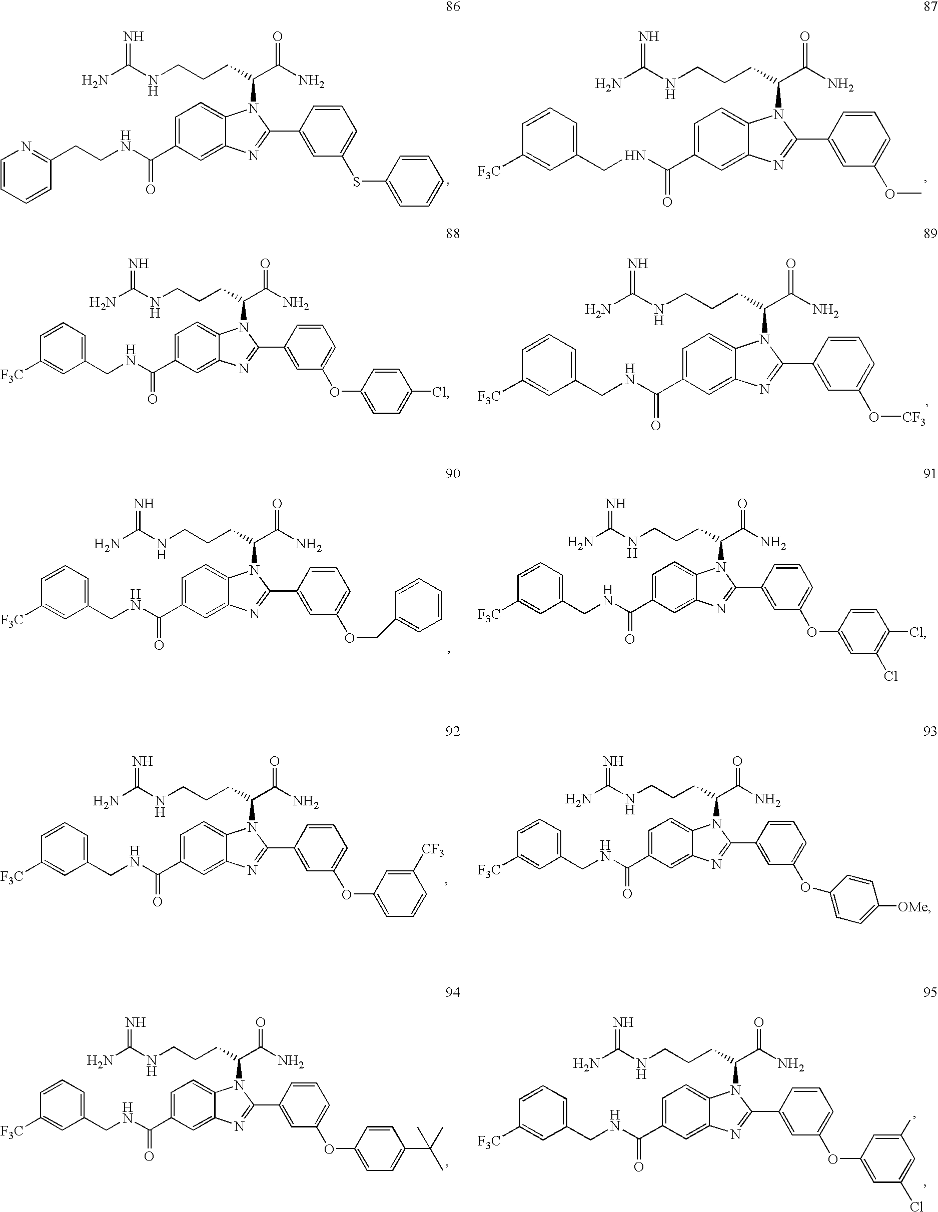

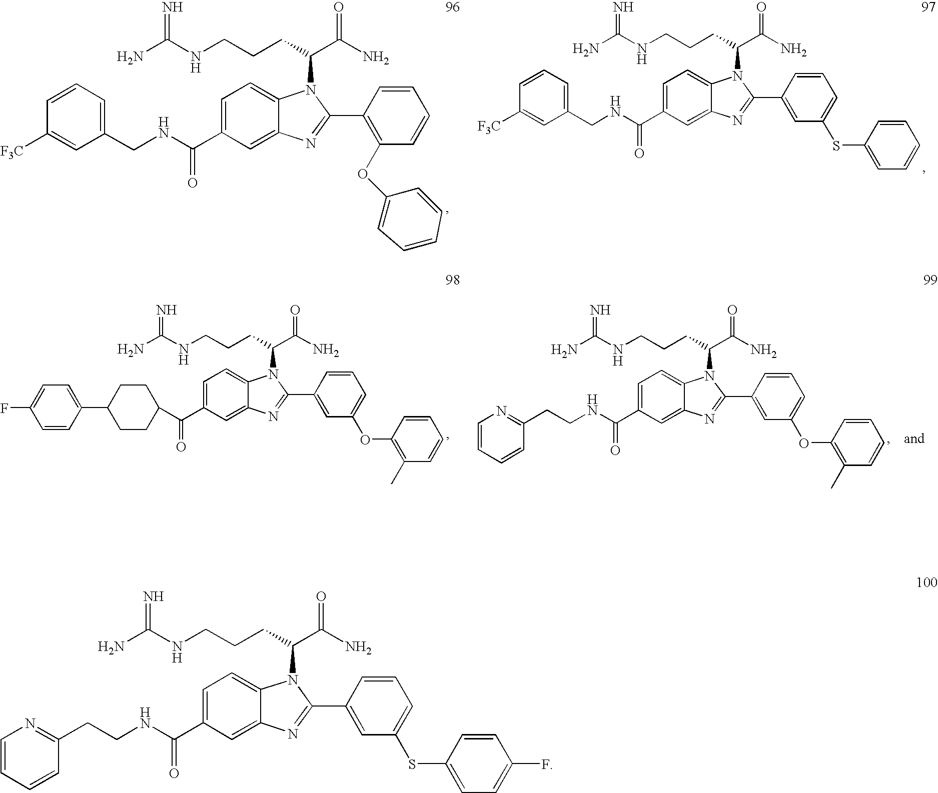

In another aspect, disclosed herein is a compound of Formula I:

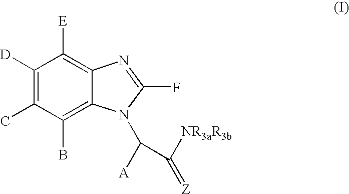

or a pharmaceutically acceptable salt or prodrug thereof, wherein:
A, B, C, E, F, and Z are each as described above;
D is selected from the group consisting of —C(=Z)NR₄ₐR_{4b}, —C(=Z)N(R₄)NR₄ₐR_{4b}, —C(R₄)═NR₄ₐ, —N(R₄)—C(=Z)R₄, and —N(R₄)—C(=Z)NR₄ₐR_{4b};
R₁, R₁ₐ, R_{1b} and R₂ are each as described above;
R₃ₐ and R_{3b} are each as described above; and
R₄, R₄ₐ, and R_{4b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl, wherein R₄ₐ and R_{4b}, taken together with the nitrogen atom to which they are attached, form a five- or six-membered optionally substituted cycloalkyl, optionally substituted heterocycloalkyl, optionally substituted aryl, or optionally substituted heteroaryl ring.

In some embodiments, R₄ₐ and R_{4b}, taken together with the nitrogen atom to which they are attached, form an optionally substituted ring selected from the group consisting of pyridine, pyrrolidine, pyrrolidinone, indazole, N-morpholine, and piperazine. In some of these embodiments, the ring is an optionally substituted morpholine or an optionally substituted piperazine.

In some embodiments, the compound of Formula I is selected from the group consisting of

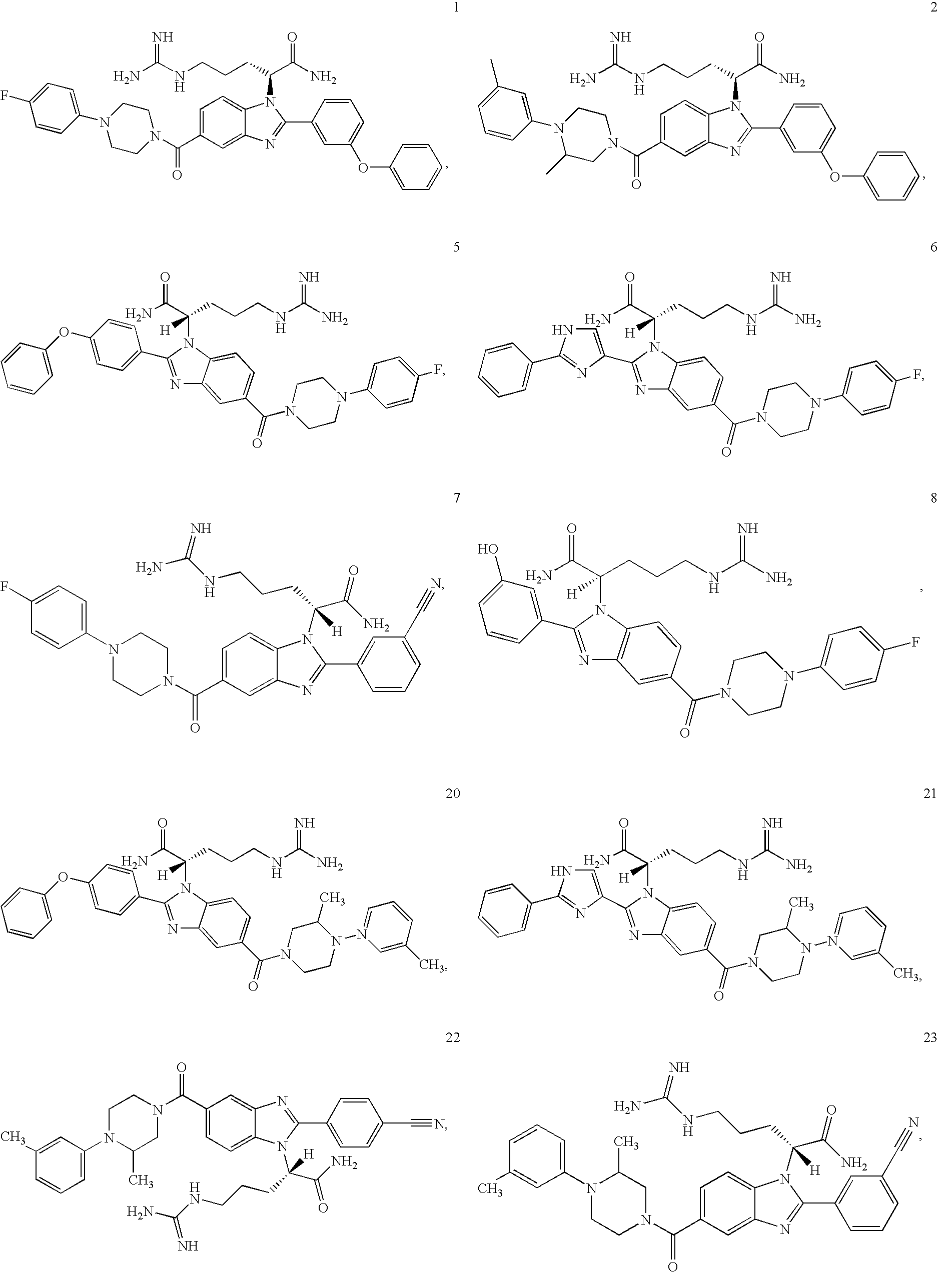

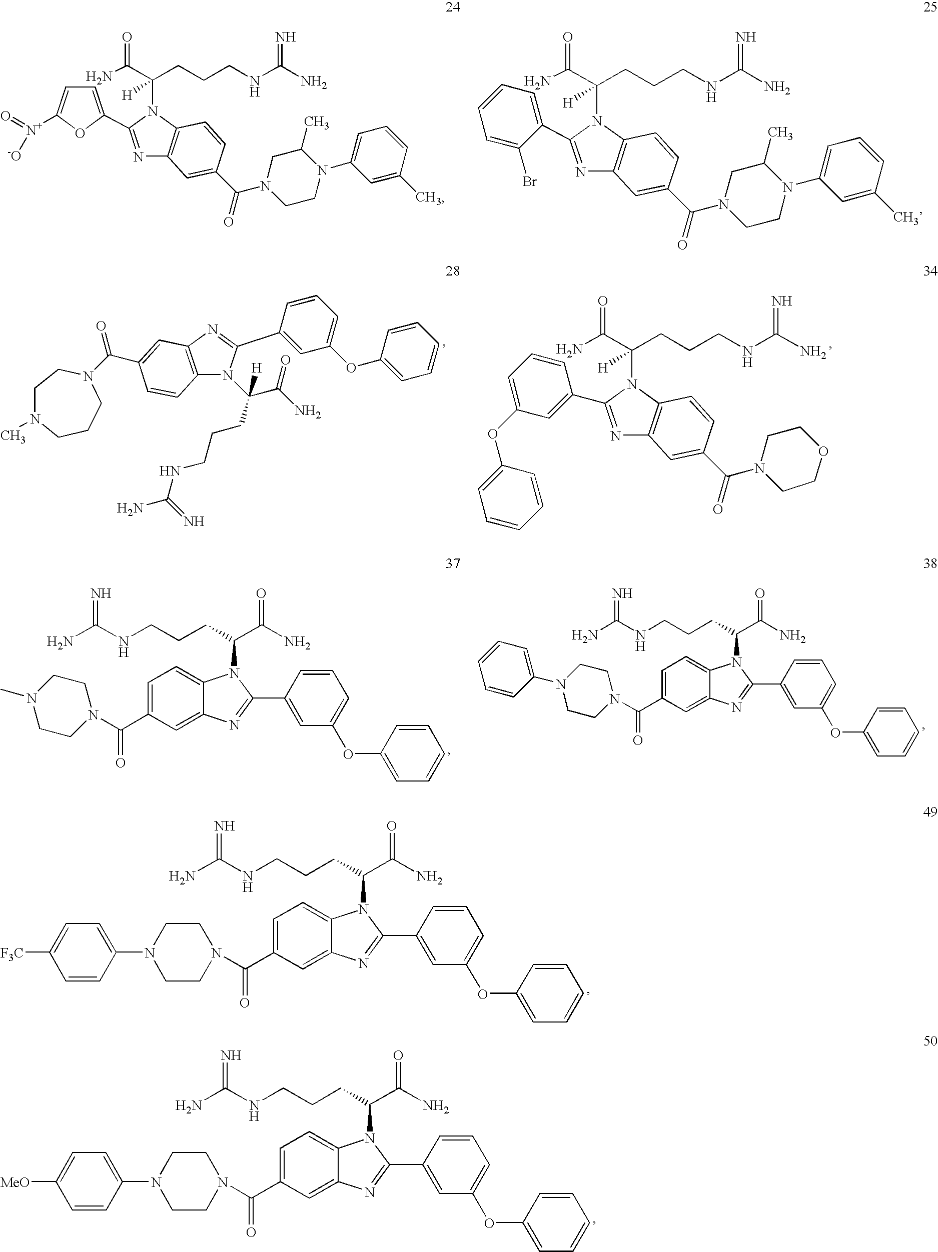

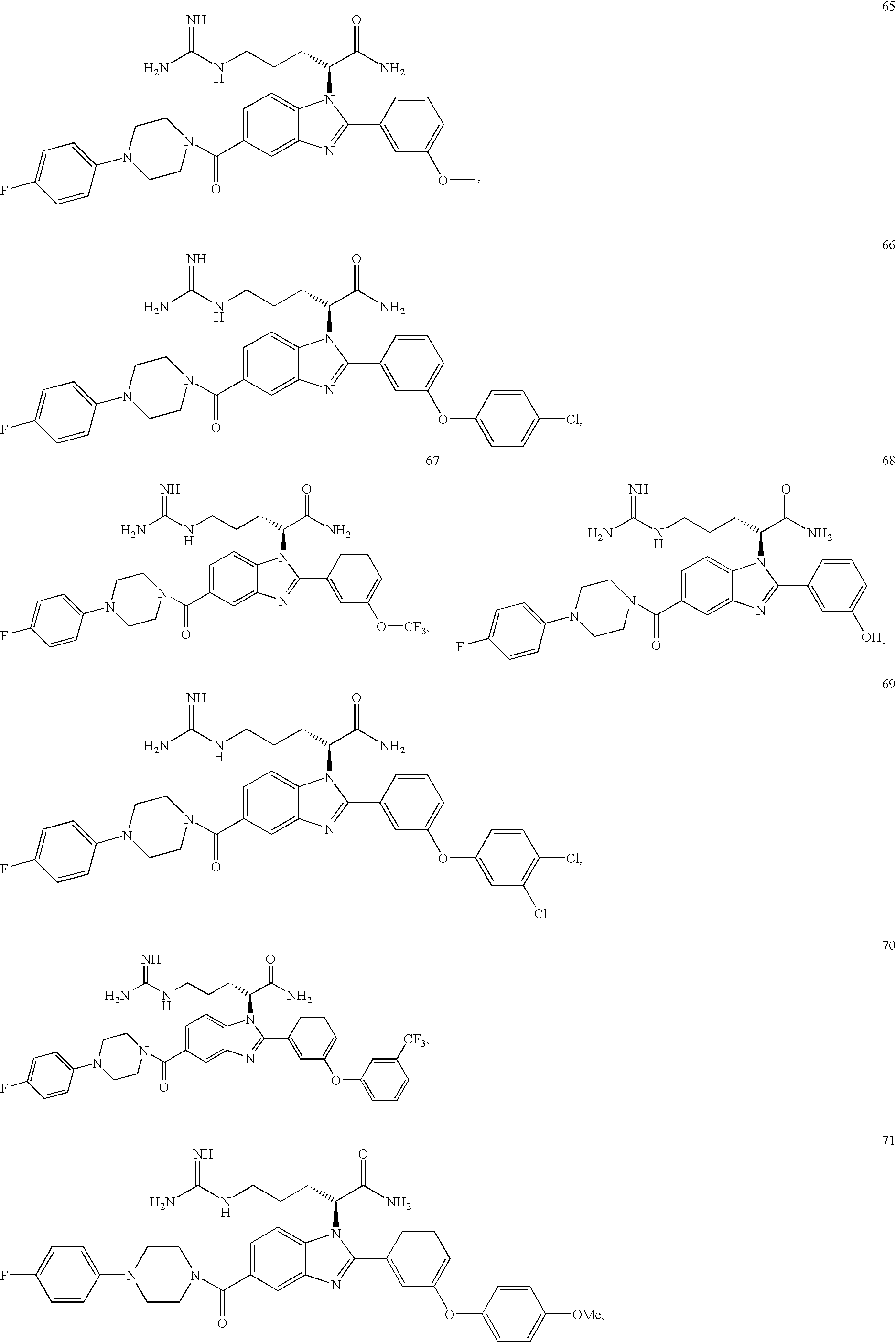

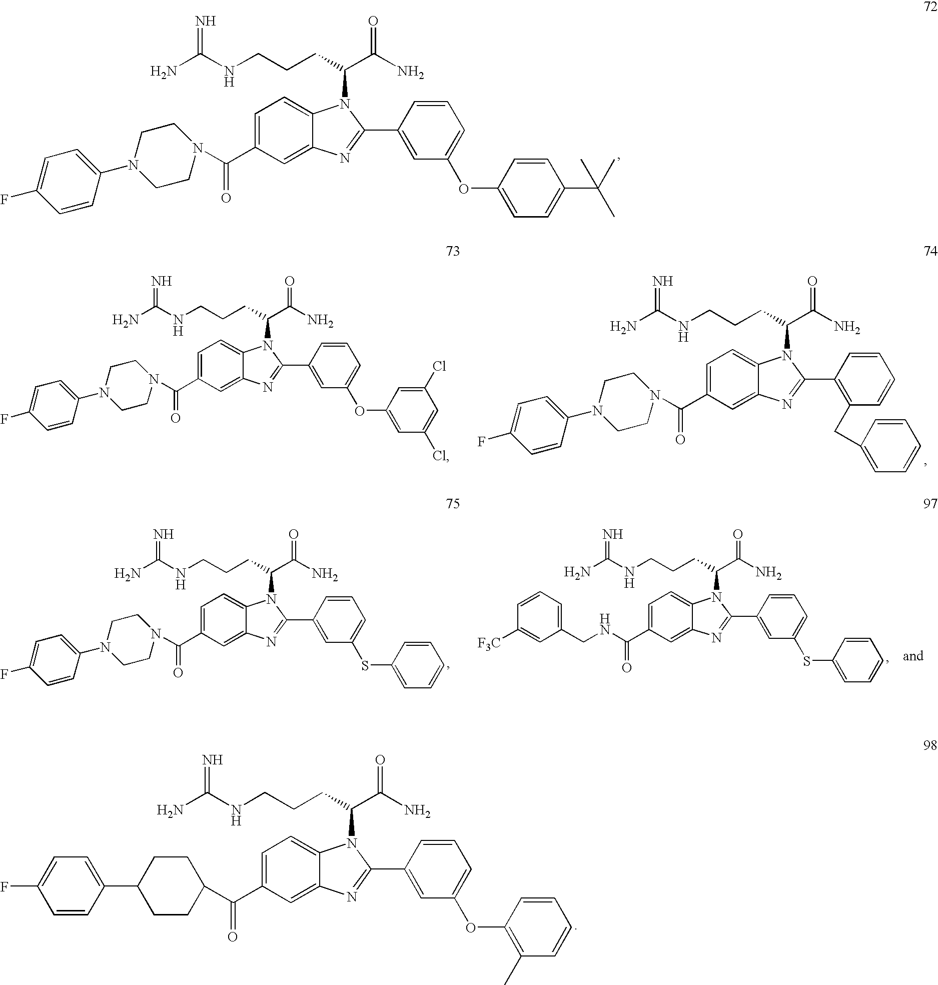

In another aspect, disclosed herein is a compound of Formula I:

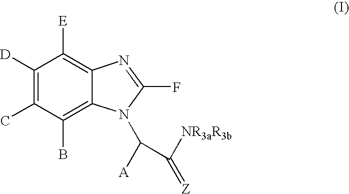

or a pharmaceutically acceptable salt or prodrug thereof, wherein:
A, B, C, E, F, and Z are each as described above;
D is selected from the group consisting of optionally substituted heteroaryl, —C(=Z)R₄, —C(=Z)OR₄, —S(O)NR₄ₐR_{4b}, —S(O)₂NR₄ₐR_{4b}, —N(R₄)—S(═O)R₄, —N(R₄)—S(═O)₂R₄, —S(O)R₄, —S(O)₂R₄, and —OC(=Z)R₄;
R₁, R₁ₐ, R_{1b} and R₂ are each as described above;
R₄, R₄ₐ, and R_{4b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl, or wherein R₄ₐ, and R_{4b}, taken together with the nitrogen atom to which they are attached, form a five- or six-membered optionally substituted cycloalkyl, optionally substituted heterocycloalkyl, optionally substituted aryl, or optionally substituted heteroaryl ring.

In another aspect, disclosed herein is a compound of Formula II:

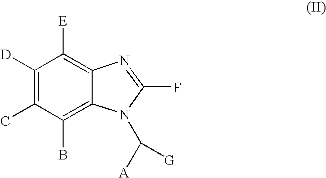

or a pharmaceutically acceptable salt or prodrug thereof, wherein:
A, B, C, E, F, and Z are each as described above;
D is selected from the group consisting of —C(=Z)NR₄ₐR_{4b}, —C(=Z)N(R₄)NR₄ₐR_{4b}, —C(R₄)═NR₄ₐ, —N(R₄)—C(=Z)R₄, and —N(R₄)—C(=Z)NR₄ₐR_{4b};
G is selected from the group consisting of hydrogen, optionally substituted heteroaryl, —C(=Z)R₁, —C(=Z)OR₁, —C(=Z)N(R₁)NR₁ₐR_{1b}, —C(R₁)C(=Z)NR₁ₐR_{1b}, —C(R₁)═NR₁ₐ, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, and —OC(=Z)R₁;
R₁, R₁ₐ, R_{1b} and R₂ are each as described above; and
R₄, R₄ₐ, and R_{4b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl.

In another aspect, disclosed herein is a compound of Formula II:

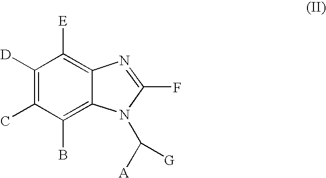

or a pharmaceutically acceptable salt or prodrug thereof, wherein:
A, B, C, E, F, and Z are each as described above;
D is selected from the group consisting of —C(=Z)NR₄ₐR_{4b}, —C(=Z)N(R₄)NR₄ₐR_{4b}, —C(R₄)═NR₄ₐ, —N(R₄)—C(=Z)R₄, and —N(R₄)—C(=Z)NR₄ₐR_{4b};
G is selected from the group consisting of hydrogen, optionally substituted heteroaryl, —C(=Z)R₁, —C(=Z)OR₁, —C(=Z)N(R₁)NR₁ₐR_{1b}, —C(R₁)C(=Z)NR₁ₐR_{1b}, —C(R₁)═NR₁ₐ, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, and —OC(=Z)R₁;
R₁, R₁ₐ, R_{1b} and R₂ are each as described above; and
R₄, R₄ₐ, and R_{4b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl, wherein R₄ₐ, and R_{4b}, taken together with the nitrogen atom to which they are attached, form a five- or six-membered optionally substituted cycloalkyl, optionally substituted heterocycloalkyl, optionally substituted aryl, or optionally substituted heteroaryl ring.

In another aspect, disclosed herein is a compound of Formula II:

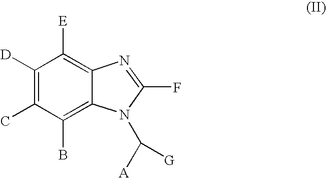

or a pharmaceutically acceptable salt or prodrug thereof, wherein:
A, B, C, E, F, and Z are each as described above;
D is selected from the group consisting of optionally substituted heteroaryl, —C(=Z)R₄, —C(=Z)OR₄, —S(O)NR₄ₐR_{4b}, —S(O)₂NR₄ₐR_{4b}, —N(R₄)—S(═O)R₄, —N(R₄)—S(═O)₂R₄, —S(O)R₄, —S(O)₂R₄, and —OC(=Z)R₄;
G is selected from the group consisting of hydrogen, optionally substituted heteroaryl, —C(=Z)R₁, —C(=Z)OR₁, —C(=Z)N(R₁)NR₁ₐR_{1b}, —C(R₁)C(=Z)NR₁ₐR_{1b}, —C(R₁)═NR₁ₐ, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, and —OC(=Z)R₁;
R₁, R₁ₐ, R_{1b} and R₂ are each as described above; and
R₄, R₄ₐ, and R_{4b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl, or wherein R₄ₐ, and R_{4b}, taken together with the nitrogen atom to which they are attached, form a five- or six-membered optionally substituted cycloalkyl, optionally substituted heterocycloalkyl, optionally substituted aryl, or optionally substituted heteroaryl ring.

### II. Method of Synthesis

In another aspect, disclosed herein is a method of synthesizing the compounds of any one of Formula I, II, or III. The following syntheses are provide by way of illustration only and are not intended, nor should they be construed, as limiting the scope of this invention in any manner whatsoever. Those skilled in the art will, based on the disclosures herein, recognize modifications to the illustrated synthetic routes as well as other synthetic routes to the compounds herein; all such routes are within the scope of this invention. More detailed synthetic procedures for each individual compound are provided in the examples, below.

Schemes 1 provides general synthetic routes to the compounds disclosed herein.

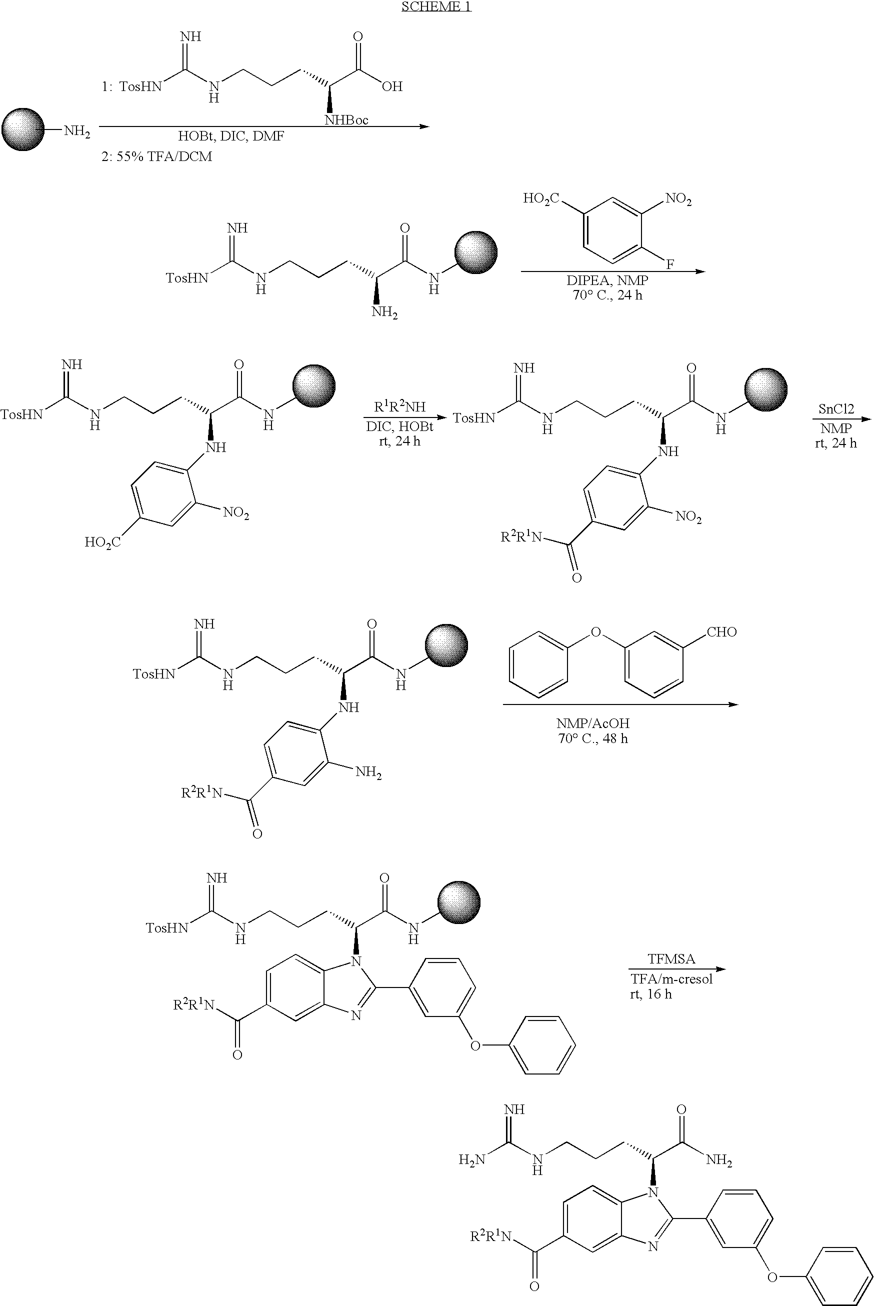

wherein

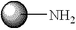

represents a solid surface, such as a functionalized resin.

As used herein, a “functionalized resin” refers to any resin to which functional groups have been appended. Such functionalized resins are well-known to those skilled in the art and include, without limitation, resins functionalized with amino, alkylhalo, formyl or hydroxy groups. Examples of functionalized resins which can serve as solid supports for immobilized solid phase synthesis are well-known in the art and include, without limitation, 4-methylbenzhydrylamine-copoly(styrene-1% divinylbenzene) (MBHA), 4-hydroxymethylphenoxymethyl-copoly(styrene-1% divinylbenzene), 4-oxymethyl-phenyl-acetamido-copoly(stryene-1% divinylbenzene) (Wang), 4-(oxymethyl)-phenylacetamido methyl (Pam), and Tentagel™, from Rapp Polymere Gmbh, trialkoxy-diphenyl-methyl ester-copoly(styrene-1% divinylbenzene)(RINK) all of which are commercially available. Other functionalized resins useful in the synthesis of the compounds of this invention will become apparent to those skilled in the art based on the disclosures herein. All such resins are within the scope of this invention.

### II. Pharmaceutical Compositions

In another aspect, disclosed herein is a pharmaceutical composition comprising a therapeutically effective amount of a compound of any one of Formula I, II, or III, and a pharmaceutically acceptable carrier, excipient, or diluent.

As used herein, a “therapeutically effective amount” refers to an amount of a compound that elicits the desired biological or medicinal response in a subject.

As used herein, a “pharmaceutical composition” refers to a mixture of a compound of this invention with other chemical components such as diluents, carriers or other excipients. A pharmaceutical composition may facilitate administration of the compound to a subject. Many techniques of administering a compound exist are known in the art, such as, without limitation, orally, intramuscularly, intraocularly, intranasally, parenterally, intravenously and topically. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The compounds of this invention can be administered to a subject per se, or in a pharmaceutical composition where they are mixed with other active ingredients as, for example, in a combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, without limitation, include oral, rectal, transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

Alternatively, one may administer the compound in a local rather than systemic manner, for example, via injection of the compound directly into the area of pain or inflammation, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured procedures well-known in the art, e.g., by means of conventional mixing, dissolving, granulating, dragee-making, levigating, emulsifying, encapsulating, entrapping or tabletting processes.

Pharmaceutical compositions for use in accordance with the present disclosure thus may be formulated in conventional manner using one or more pharmaceutically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may be used.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### IV. Activity of the Compounds

In another aspect, disclosed herein is a method of modulating the activity of an MrgX1 or an MrgX2 receptor, comprising: contacting the MrgX1 or the MrgX2 receptor with a compound of any one of Formula I, II, or III, as described herein; detecting changes in the activity of the receptor; and/or comparing the activity of the receptor after the contacting to the activity of the receptor before the contacting. In some embodiments, the compound of any one of Formula I, II, or III is an agonist of the MrgX receptors. In other embodiments, the compound of any one of Formula I, II, or III is an antagonist of the MrgX receptors MrgX receptors. In yet other embodiments, the compound of any one of Formula I, II, or III is an inverse agonist of the MrgX receptors. In still other embodiments, the compound of any one of Formula I, II, or III is a partial agonist of the MrgX receptors.

As used herein, to “modulate” the activity of an MrgX receptor means either to activate it, i.e., to increase its cellular function over the base level measured in the particular environment in which it is found, or deactivate it, i.e., decrease its cellular function to less than the measured base level in the environment in which it is found and/or render it unable to perform its cellular function at all even in the presence of a natural binding partner. A natural binding partner is an endogenous molecule that is an agonist for the receptor.

As used herein, to “detect” changes in the activity of an MrgX receptor refers to the process of analyzing the result of an experiment using whatever analytical techniques are best suited to the particular situation. In some cases simple visual observation may suffice, in other cases the use of a microscope, visual or UV light analyzer or specific bioassays may be required. The proper selection of analytical tools and techniques to detect changes in the activity of MrgX receptors are well-known and will be apparent to those skilled in the art based on the disclosures herein.

As used herein, an “agonist” refers to a compound that binds to a receptor to from a complex that elicits the full pharmacological response associated with that particular receptor.

As used herein, “partial agonist” refers to a compound that has an affinity for a receptor but, unlike a full agonist, when bound to the receptor it elicits only a small degree of the pharmacological response normally associated with the receptor even if a large fraction of receptors are occupied by the compound.

As used herein, “inverse agonist” refers to a compound that inhibits the constitutive activity of a receptor such that the compound is not technically an antagonist but, rather, is an agonist with negative instrinsic activity.

As used herein, “antagonist” refers to a compound that binds to a receptor to form a complex that does not give rise to any response, as if the receptor were unoccupied. An antagonist often bind essentially irreversibly to the receptor, effectively eliminating the activity of the receptor permanently or at least until the antagonist is metabolized or otherwise removed by biological process.

In some embodiments, the above receptor is contacted with the compound of any one of Formula I, II, or III in vivo, e.g., when the receptor is in a tissue or in an animal. In other embodiments, the above receptor is contacted with the compound of any one of Formula I, II, or III in vitro, e.g., in an assay, or when the receptor is in an intact cell or in a plurality of cells.

In some embodiments, the compound of any one of Formula I, II, or III selectively modulates the MrgX1 or the MrgX2 receptor activity relative to other receptors that mediate analgesia. In some embodiments, the other receptors that mediate analgesia comprise the opioid receptors.

Throughout the present disclosure, the MrgX1 or the MrgX2 receptor can be selected from the group consisting of a human MrgX1 receptor, a simian MrgX1 receptor, a human MrgX2 receptor, and a simian MrgX2 receptor.

In another aspect, disclosed herein is a method of alleviating acute, chronic and neuropathic pain in a subject, comprising: identifying a subject in need thereof, and administering to the subject a therapeutically effective amount of a compound of any one of Formula I, II, or III. In some embodiments, the subject is a patient.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes; and, in particular, humans.

As used herein, a “patient” refers to a subject that is being treated by a medical professional such as an M.D. or a D.V.M. to attempt to cure, or at least ameliorate the effects of, a particular disease or disorder or to prevent the disease or disorder from occurring in the first place.

In some embodiments, the pain, whether acute pain, chronic pain or neuropathic pain is caused by trauma, by diseases such as diabetes, herpes zoster (shingles), irritable bowel syndrome or late-stage cancer, by acute and chronic inflammation, by arthritis, by amputation, by physical trauma, or by chemical injury, for example, as an unintended consequence of drug therapies including, but not limited to, the antiviral drugs.

In another aspect, disclosed herein is a method of identifying a compound that modulates the activity of an MrgX1 or an MrgX2 receptor, comprising: contacting the MrgX1 or the MrgX2 receptor with a plurality of compounds of any one of Formula I, II, or III one at a time; comparing the activity of the receptor after the contacting with each compound of any one of Formula I, II, or III to the activity of the receptor before the contacting; and selecting a compound of any one of Formula I, II, or III that changes the activity of the receptor after the contacting.

In some embodiments, the receptor is located within a cell, while in other embodiments, the receptor is located within a plurality of cells. In further embodiments, the receptor is located within a cell extract that expresses the receptor, e.g., a cell extract that contains the genetic code for any of the MrgX receptors.

Contacting a cell or plurality of cells may comprise incubating the cell(s) with the test compound. The cell(s) may be engineered to over-express the receptor. The assay may further comprise the addition of an known agonist to the test milieu to assist in differentiating an antagonist from an inverse agonist. In general, if the activity of the receptor is increased, the compound is an agonist, if the basal activity of the receptor, as measured before any compound is added, is decreased, the compound is likely an inverse agonist while if the receptor is inactivated, the compound is an antagonist.

In another aspect, disclosed herein is a method of identifying a compound effective for the treatment of pain, comprising: contacting a compound of any one of Formula I, II, or III with a receptor selected from the group consisting of a human MrgX1 receptor, a simian MrgX1 receptor, a human MrgX2 receptor, and a simian MrgX2 receptor; comparing the activity of the receptor after the contacting with each compound of any one of Formula I, II, or III to the activity of the receptor before the contacting; and selecting a compound of any one of Formula I, II, or III that changes the activity of the receptor after the contacting.

### EXAMPLES

The following examples are provided by way of illustration only and are not intended, nor should they be construed, as limiting the scope of this invention in any manner whatsoever.

### ABBREVIATIONS

MBHA: 4-methylbenzhydrylamine

DMF: dimethylformamide

HOBt: 1-hydroxybenzotriazole

NMP: N-methylpyrrolidone

Boc: tert-butoxycarbonyl

DIC: N,N′-diisopropylcarbodiimide

TFA: trifluoroacetic acid

DIPEA: diisopropylethylamine

DCM dichloromethane

TFMSA: trifluoromethanesulfonic acid

### Example 1

### Synthetic Chemistry

### General Analytical LC-MS Procedure

### Procedure 1 (AP1):

The analysis was performed on a combined prep/analytical Waters/Micromass system consisting of a ZMD single quadropole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammoniumacetate in water, buffer B: 10 mM ammoniuacetate in acetonitrile/water 95/5. A gradient was run from 10% B to 100% B in 10 min, stay at 100% B for 1 min, re-equilibrate for 6 min. System was operated at 1 ml/min.

### Procedure 2 (AP2):

The analysis was performed on a combined prep/analytical Waters/Micromass system consisting of a ZMD single quadropole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammoniumacetate in water, buffer B: 10 mM ammoniuacetate in acetonitrile/water 95/5. A gradient was run from 30% B to 100% B in 7 min, stay at 100% B for 1 min, re-equilibrate for 5.5 min. System was operated at 1 ml/min.

### General Preparative HPLC

Preparative purification was performed on Waters Delta 4000 preparative system, Water 2487 dual absorbance detector, and Waters Fraction collector II. The column used was a Luna 15 μm C18, 250×21.2 mm. The following mobile phases were used: a) H₂O/MeCN 9:1 ammonium acetate buffer (25 nM) and b) H₂O/MeCN 1:4 ammonium acetate buffer (25 nM).

### General Synthetic Procedures

### General Synthetic Procedure (GP1)

### (α¹R)-α-[3-[(aminoiminomethyl)amino]propyl]-5-[[4-(4-fluorophenyl)-1-piperazinyl]carbonyl]-2-(3-phenoxyphenyl)-1H-benzimidazole-1-acetamide (1)

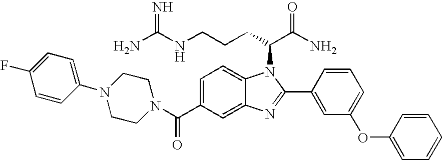

### Step 1: Coupling of Boc-Arg(Tos)-OH to MBHA Resin

4 g of MBHA resin (1.16 mmol/g) was placed in a Teflon vial fitted with a frit. DCM was added and the resin was allowed to swell for two hours at rt. Then the DCM was filtered off. Boc-Arg(Tos)-OH (19.9 g, 46.4 mmol), HOBt (6.3 g, 46.4 mmol) and DIC (8.8 g, 68.6 mmol) were dissolved in DMF (80 mL) and added to the resin and shaken at rt for 24 hours.

Wash: The resin was alternatively washed with DMF (80 mL) and MeOH (80 mL) for three circles, then with DCM (80 mL) and MeOH (80 mL).

After two hours drying 55% TFA in DCM (80 mL) was added to the resin, and shaken at rt for 40 min.

Wash: The resin was washed with DCM (80 mL) three times, then 5% DIPEA in DCM (80 mL) twice followed by MeOH (80 mL).

### Step 2: N-Alkylation with 4-fluoro-3-nitrobenzoic acid

4-Fluoro-3-nitrobenzoic acid (8.6 g, 46.4 mmol) and DIPEA (6.0 g, 46.4 mmol) were taken up in NMP (80 mL), added to the resin and shaken on a shaker for 24 h at 70° C.

Wash: The resin was alternatively washed with DMF (80 mL) and MeOH (80 mL) for three circles, then with DCM (80 mL) and MeOH (80 mL).

### Step 3: Coupling of 1-(4-fluorophenyl)piperazine with resin bound carboxylic acid

1-(4-Fluorophenyl)piperazine (8.4 g, 46.4 mmol), HOBt (6.3 g, 46.4 mmol) and DIC (8.8 g, 68.6 mmol) were dissolved in DMF (80 mL), added to the resin and shaken on a shaker at rt for 24 hours.

Wash: The resin was alternatively washed with DMF (80 mL) and MeOH (80 mL) for three circles, then with DCM (80 mL) and MeOH (80 mL).

### Step 4: Reduction of the Nitro Group

Stannous chloride, dihydrate (36 g, 160 mmol) was taken up in NMP (80 mL), added to the resin and shaken on a shaker at rt for 24 hours.

Wash: The resin was washed with DMF (80 mL) four times, then 5% DIPEA in DCM (80 mL) four times, MeOH (80 mL) twice, DMF (80 mL), MeOH (80 mL), DCM (80 mL) twice followed by MeOH (80 mL) twice.

### Step 5: Formation of benzimidazole through reaction with 3-phenoxy benzaldehyde

3-Phenoxybenzaldehyde (9.2 g, 46.4 mmol) was taken up in AcOH (40 mL) and NMP (40 mL), added to the resin and shakenon a shaker for 28 hours at 70° C.

Wash: The resin was washed with DMF (80 mL) three times, then MeOH (80 mL) twice, DCM (80 mL) twice followed by MeOH (80 mL) twice.

### Step 6: Cleavage from the Resin

TMSOTf (8 mL), TFA (28 mL) and m-cresol (5 mL) was mixed and added to the resin. Left on a shaker for 24 hours at rt. The crude cleavage product was precipitated with ether, the ether was decanted and the solid washed twice with ether (400 mL), then taken up in MeOH and concentrated in vacuo.

The crude was purified by prep HPLC to yield the title compound. UV/MS: 93/88.

hu 1H NMR (400 MHz, MeOD) δ: 7.83 (bs, 1H), 7.70 (d, J=8.4 Hz, 1H), 7.61 (t, J=8.0 Hz, 1H), 7.45-7.40 (m, 4H), 7.30 (bt, J=2.0 Hz, 1H), 7.26 (dd, J=8.2 and 2.1 Hz, 1H), 7.20 (t, J=7.4 Hz, 1H), 7.11 (bd, J=8.0 Hz, 2H), 7.01-6.99 (m, 4H), 5.17 (t, J=6.6 Hz, 1H), 3.95-3.71 (m, 4H), 3.27-3.11 (m, 4H), 2.97-2.92 (m, 2H), 2.34-2.30 (m, 2H), 1.23-1.16 (m, 1H), 0.90-0.82 (m, 1H).

### (α¹R)-α-[3-[(aminoiminomethyl)amino]propyl]-5-[[3-methyl-4-(3-methylphenyl)-1-piperazinyl]carbonyl]-2-(3-phenoxyphenyl)-1H-benzimidazole-1-acetamide (2)

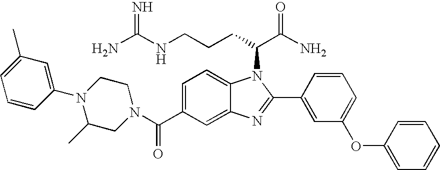

Prepared according to GP1.

### (α¹R)-α-[3-[(aminoiminomethyl)amino]propyl]-2-(3-phenoxyphenyl)-5-[[[2-(2-pyridinyl)ethyl]amino]carbonyl]-1H-benzimidazole-1-acetamide (3)

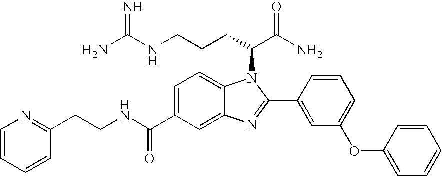

Prepared according to GP1.

### (α¹R)-α-[3-[(aminoiminomethyl)amino]propyl]-5-[[methyl[2-(2-pyridinyl)ethyl]amino]carbonyl]-2-(3-phenoxyphenyl)-1H-benzimidazole-1-acetamide (4)

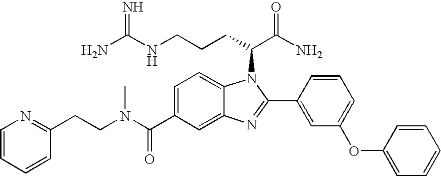

Prepared according to GP1. UV/MS: 96/84.

¹H NMR (400 MHz, MeOD) δ: 8.50 (bd, J=4.5 Hz, 1H), 8.16 (d, J=1.5 Hz, 1H), 7.80-7.75 (m, 2H), 7.65-7.59 (m, 2H), 7.44-7.37 (m, 4H), 7.30-7.09 (m, 6H), 5.15 (t, J=8.8 Hz, 1H), 3.78 (t, J=7.3 Hz, 2H), 3.14 (t, J=7.3 Hz, 2H), 2.97-2.91 (m, 2H), 2.35-2.29 (m, 2H), 1.21-1.13 (m, 1H), 0.87-0.81 (m, 1H).

### 5-guanidino-2-(5-(4-methylpiperazine-1-carbonyl)-2-(3-phenoxyphenyl)-1H-benzo[d]imidazol-1-yl)pentanamide (37)

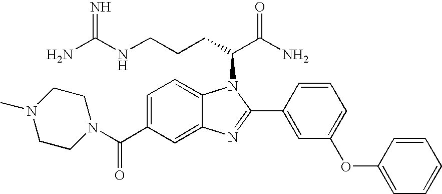

Prepared according to GP1. HPLC Rₜ: 3.28 min.

### 5-guanidino-2-(2-(3-phenoxyphenyl)-5-(4-phenylpiperazine-1-carbonyl)-1H-benzo[d]imidazol-1-yl)pentanamide (38)

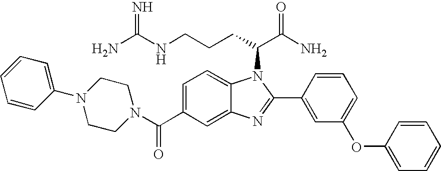

Prepared according to GP1. HPLC Rₜ: 4.55 min.

### 1-(1-amino-5-guanidino-1-oxopentan-2-yl)-N-(3-methoxyphenyl)-2-(3-phenoxyphenyl)-1H-benzo[d]imidazole-5-carboxamide (39)

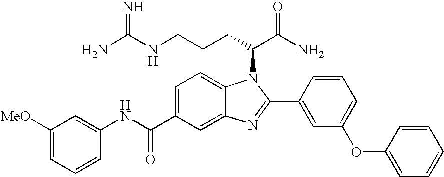

Prepared according to GP1. HPLC Rₜ: 4.47 min.

### 1-(1-amino-5-guanidino-1-oxopentan-2-yl)-N-(4-(dimethylamino)phenyl)-2-(3-phenoxyphenyl)-1H-benzo[d]imidazole-5-carboxamide (40)

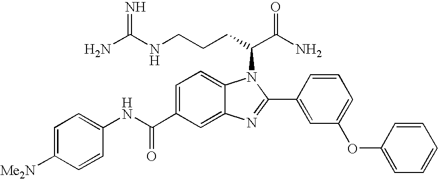

Prepared according to GP1. HPLC Rₜ: 4.40 min.

### 1-(1-amino-5-guanidino-1-oxopentan-2-yl)-N-(2-methoxyphenyl)-2-(3-phenoxyphenyl)-1H-benzo[d]imidazole-5-carboxamide (41)

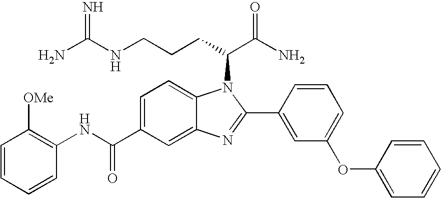

Prepared according to GP1. HPLC Rₜ: 4.47 min.

### 1-(1-amino-5-guanidino-1-oxopentan-2-yl)-2-(3-phenoxyphenyl)-N-(pyridin-2-yl)-1H-benzo[d]imidazole-5-carboxamide (42)

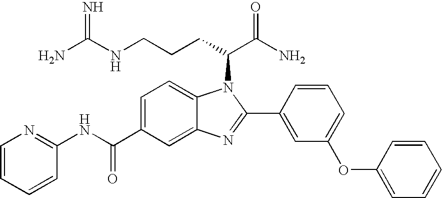

Prepared according to GP1. HPLC Rₜ: 4.01 min.

### 1-(1-amino-5-guanidino-1-oxopentan-2-yl)-2-(3-phenoxyphenyl)-N-(pyridin-3-yl)-1H-benzo[d]imidazole-5-carboxamide (43)

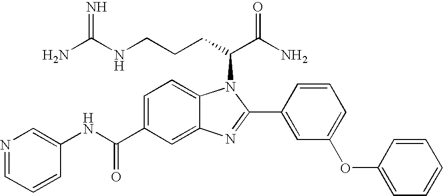

Prepared according to GP1. HPLC Rₜ: 3.76 min.

### 1-(1-amino-5-guanidino-1-oxopentan-2-yl)-2-(3-phenoxyphenyl)-N-(pyridin-4-yl)-1H-benzo[d]imidazole-5-carboxamide (44)

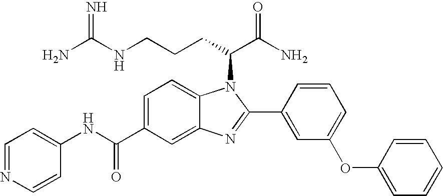

Prepared according to GP1. HPLC Rₜ: 3.72 min.

### 1-(1-amino-5-guanidino-1-oxopentan-2-yl)-2-(3-phenoxyphenyl)-N-(pyridin-3-ylmethyl)-1H-benzo[d]imidazole-5-carboxamide (45)

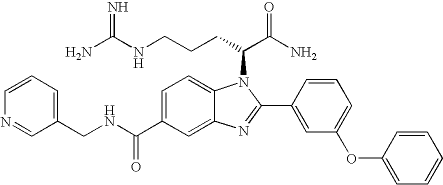

Prepared according to GP1. HPLC Rₜ: 3.61 min.

### 1-(1-amino-5-guanidino-1-oxopentan-2-yl)-2-(3-phenoxyphenyl)-N-(pyridin-2-ylmethyl)-1H-benzo[d]imidazole-5-carboxamide (46)

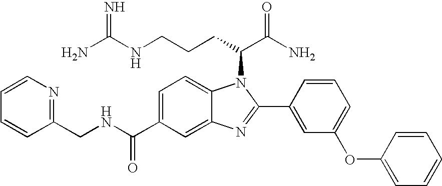

Prepared according to GP1. HPLC Rₜ: 3.65 min.

### 1-(1-amino-5-guanidino-1-oxopentan-2-yl)-2-(3-phenoxyphenyl)-N-(pyridin-4-ylmethyl)-1H-benzo[d]imidazole-5-carboxamide (47)

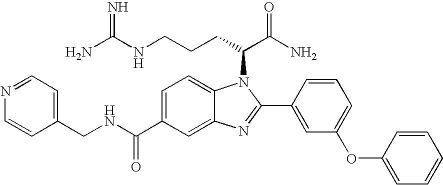

Prepared according to GP1. HPLC Rₜ: 3.50 min.

### 2-(5-(4-(3-fluorophenyl)piperazine-1-carbonyl)-2-(3-phenoxyphenyl)-1H-benzo[d]imidazol-1-yl)-5-guanidinopentanamide (48)

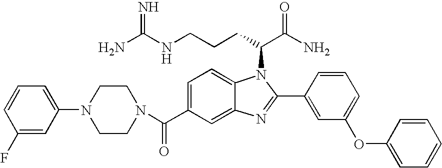

Prepared according to GP1. HPLC Rₜ: 4.82 min.

### 5-guanidino-2-(2-(3-phenoxyphenyl)-5-(4-(4-(trifluoromethyl)phenyl)piperazine-1-carbonyl)-1H-benzo[d]imidazol-1-yl)pentanamide (49)

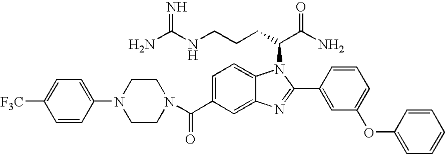

Prepared according to GP1. HPLC Rₜ: 5.32 min.

### 5-guanidino-2-(5-(4-(4-methoxyphenyl)piperazine-1-carbonyl)-2-(3-phenoxyphenyl)-1H-benzo[d]imidazol-1-yl)pentanamide (50)

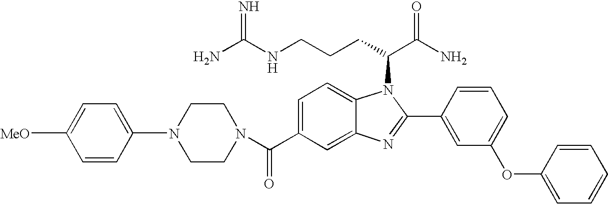

Prepared according to GP1. HPLC Rₜ: 4.47 min.

### 1-(1-amino-5-guanidino-1-oxopentan-2-yl)-2-(3-phenoxyphenyl)-N-(2-(pyridin-3-yl)ethyl)-1H-benzo[d]imidazole-5-carboxamide (51)

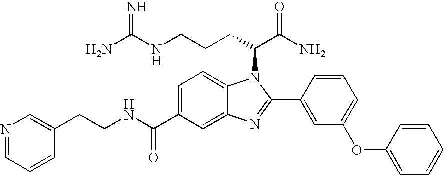

Prepared according to GP1. HPLC Rₜ: 3.63 min.

### 1-(1-amino-5-guanidino-1-oxopentan-2-yl)-2-(3-phenoxyphenyl)-N-(2-(pyridin-4-yl)ethyl)-1H-benzo[d]imidazole-5-carboxamide (52)

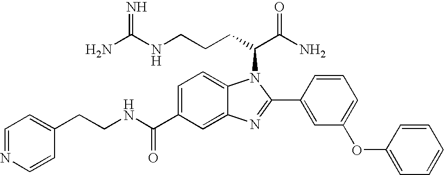

Prepared according to GP1. HPLC Rₜ: 3.63 min.

### 1-(1-amino-5-guanidino-1-oxopentan-2-yl)-N-(4-tert-butylphenyl)-2-(3-phenoxyphenyl)-1H-benzo[d]imidazole-5-carboxamide (53)

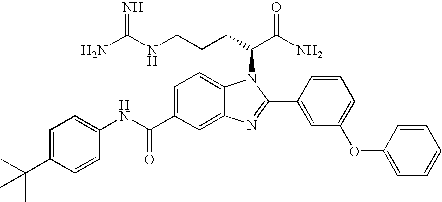

Prepared according to GP1. HPLC Rₜ: 5.43 min.

### 1-(1-amino-5-guanidino-1-oxopentan-2-yl)-2-(3-methoxyphenyl)-N-(4-methoxyphenyl)-1H-benzo[d]imidazole-5-carboxamide (54)

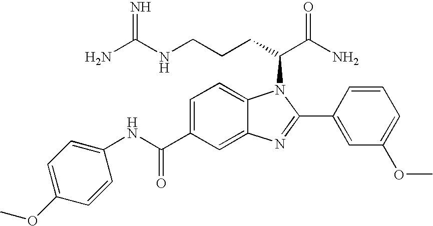

Prepared according to GP1. HPLC Rₜ: 3.41 min.

### 1-(1-amino-5-guanidino-1-oxopentan-2-yl)-2-(3-(4-chlorophenoxy)phenyl)-N-(4-methoxyphenyl)-1H-benzo[d]imidazole-5-carboxamide (55)

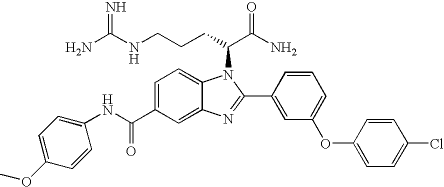

Prepared according to GP1. HPLC Rₜ: 4.71 min.

### 1-(1-amino-5-guanidino-1-oxopentan-2-yl)-N-(4-methoxyphenyl)-2-(3-(trifluoromethoxy)phenyl)-1H-benzo[d]imidazole-5-carboxamide (56)

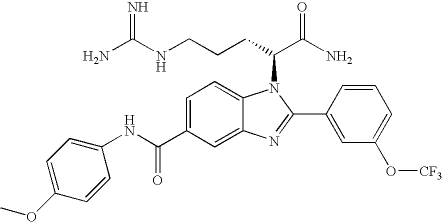

Prepared according to GP1. HPLC Rₜ: 4.05 min.

### 1-(1-amino-5-guanidino-1-oxopentan-2-yl)-2-(3-hydroxyphenyl)-N-(4-methoxyphenyl)-1H-benzo[d]imidazole-5-carboxamide (57)

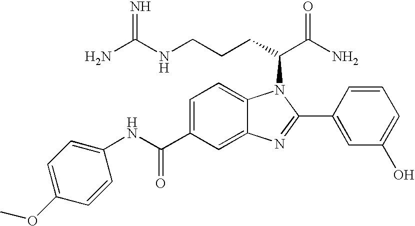

Prepared according to GP1. HPLC Rₜ: 3.06 min.

### 1-(1-amino-5-guanidino-1-oxopentan-2-yl)-2-(3-(3,4-dichlorophenoxy)phenyl)-N-(4-methoxyphenyl)-1H-benzo[d]imidazole-5-carboxamide (58)

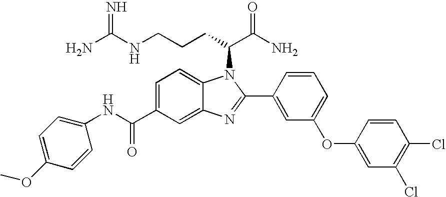

Prepared according to GP1. HPLC Rₜ: 4.88 min.

### 1-(1-amino-5-guanidino-1-oxopentan-2-yl)-N-(4-methoxyphenyl)-2-(3-(3-(trifluoromethyl)phenoxy)phenyl)-1H-benzo[d]imidazole-5-carboxamide (59)

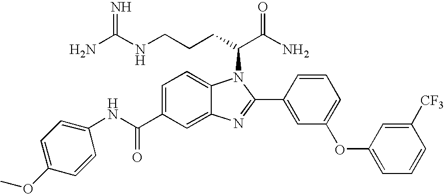

Prepared according to GP1. HPLC Rₜ: 4.84 min.

### 1-(1-amino-5-guanidino-1-oxopentan-2-yl)-2-(4-(4-methoxyphenoxy)phenyl)-N-(4-methoxyphenyl)-1H-benzo[d]imidazole-5-carboxamide (60)

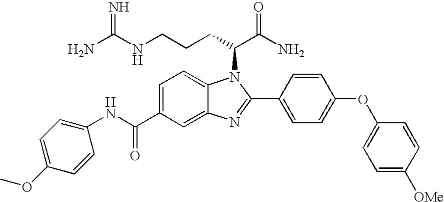

Prepared according to GP1. HPLC Rₜ: 4.25 min.

### 1-(1-amino-5-guanidino-1-oxopentan-2-yl)-2-(3-(4-tert-butylphenoxy)phenyl)-N-(4-methoxyphenyl)-1H-benzo[d]imidazole-5-carboxamide (61)

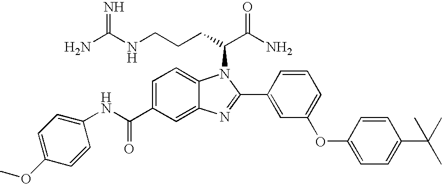

Prepared according to GP1. HPLC Rₜ: 5.37 min.

### 1-(1-amino-5-guanidino-1-oxopentan-2-yl)-2-(3-(3,5-dichlorophenoxy)phenyl)-N-(4-methoxyphenyl)-1H-benzo[d]imidazole-5-carboxamide (62)

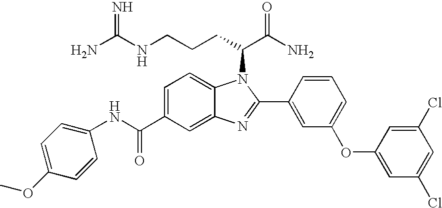

Prepared according to GP1. HPLC Rₜ: 4.91 min.

### 1-(1-amino-5-guanidino-1-oxopentan-2-yl)-N-(4-methoxyphenyl)-2-(2-phenoxyphenyl)-1H-benzo[d]imidazole-5-carboxamide (63)

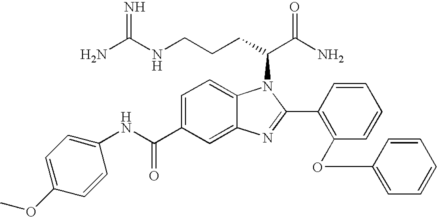

Prepared according to GP1. HPLC Rₜ: 4.07 min.

### 1-(1-amino-5-guanidino-1-oxopentan-2-yl)-N-(4-methoxyphenyl)-2-(3-(phenylthio)phenyl)-1H-benzo[d]imidazole-5-carboxamide (64)

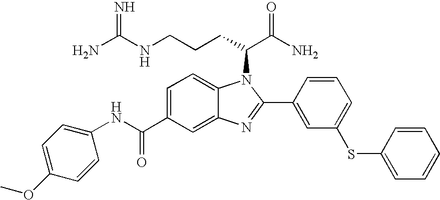

Prepared according to GP1. HPLC Rₜ: 4.64 min.

### 2-(5-(4-(4-fluorophenyl)piperazine-1-carbonyl)-2-(3-methoxyphenyl)-1H-benzo[d]imidazol-1-yl)-5-guanidinopentanamide (65)

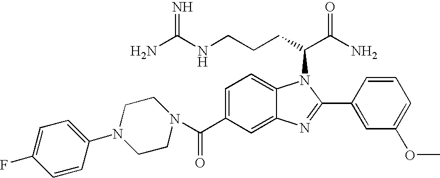

Prepared according to GP1. HPLC Rₜ: 3.89 min.

### 2-(2-(3-(4-chlorophenoxy)phenyl)-5-(4-(4-fluorophenyl)piperazine-1-carbonyl)-1H-benzo[d]imidazol-1-yl)-5-guanidinopentanamide (66)

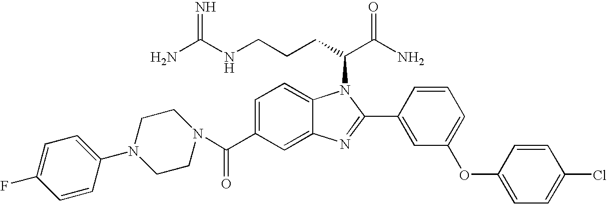

Prepared according to GP1. HPLC Rₜ: 4.91 min.

### 2-(5-(4-(4-fluorophenyl)piperazine-1-carbonyl)-2-(3-(trifluoromethoxy)phenyl)-1H-benzo[d]imidazol-1-yl)-5-guanidinopentanamide (67)

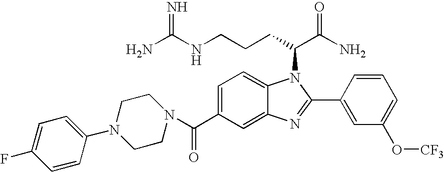

Prepared according to GP1. HPLC Rₜ: 4.36 min.

### 2-(5-(4-(4-fluorophenyl)piperazine-1-carbonyl)-2-(3-hydroxyphenyl)-1H-benzo[d]imidazol-1-yl)-5-guanidinopentanamide (68)

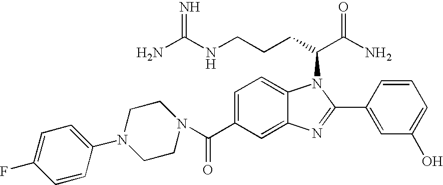

Prepared according to GP1. HPLC Rₜ: 3.59 min.

### 2-(2-(3-(3,4-dichlorophenoxy)phenyl)-5-(4-(4-fluorophenyl)piperazine-1-carbonyl)-1H-benzo[d]imidazol-1-yl)-5-guanidinopentanamide (69)

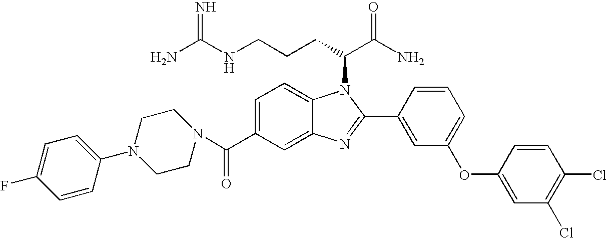

Prepared according to GP1. HPLC Rₜ: 5.19 min.

### 2-(5-(4-(4-fluorophenyl)piperazine-1-carbonyl)-2-(3-(3-(trifluoromethyl)phenoxy)phenyl)-1H-benzo[d]imidazol-1-yl)-5-guanidinopentanamide (70)

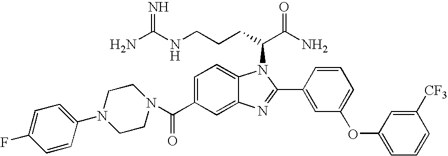

Prepared according to GP1. HPLC Rₜ: 5.10 min.

### 2-(5-(4-(4-fluorophenyl)piperazine-1-carbonyl)-2-(3-(4-methoxyphenoxy)phenyl)-1H-benzo[d]imidazol-1-yl)-5-guanidinopentanamide (71)

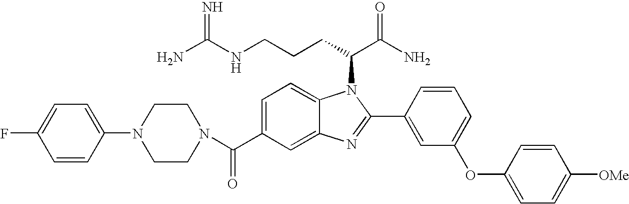

Prepared according to GP1. HPLC Rₜ: 4.62 min.

### 2-(2-(3-(4-tert-butylphenoxy)phenyl)-5-(4-(4-fluorophenyl)piperazine-1-carbonyl)-1H-benzo[d]imidazol-1-yl)-5-guanidinopentanamide (72)

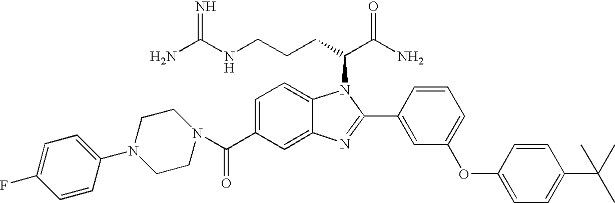

Prepared according to GP1. HPLC Rₜ: 4.73 min.

### 2-(2-(3-(3,5-dichlorophenoxy)phenyl)-5-(4-(4-fluorophenyl)piperazine-1-carbonyl)-1H-benzo[d]imidazol-1-yl)-5-guanidinopentanamide (73)

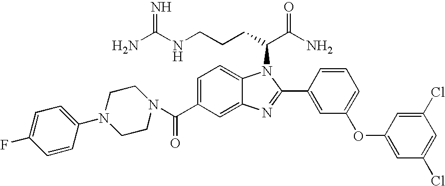

Prepared according to GP1. HPLC Rₜ: 5.23 min.

### 2-(5-(4-(4-fluorophenyl)piperazine-1-carbonyl)-2-(2-phenoxyphenyl)-1H-benzo[d]imidazol-1-yl)-5-guanidinopentanamide (74)

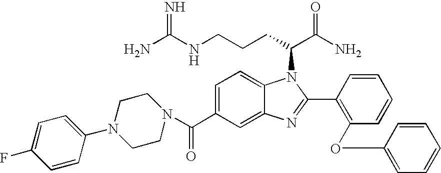

Prepared according to GP1. HPLC Rₜ: 4.40 min.

### 2-(5-(4-(4-fluorophenyl)piperazine-1-carbonyl)-2-(3-(phenylthio)phenyl)-1H-benzo[d]imidazol-1-yl)-5-guanidinopentanamide (75)

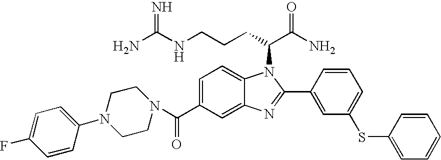

Prepared according to GP1. HPLC Rₜ: 4.73 min.

### 1-(1-amino-5-guanidino-1-oxopentan-2-yl)-2-(3-methoxyphenyl)-N-(2-(pyridin-2-yl)ethyl)-1H-benzo[d]imidazole-5-carboxamide (76)

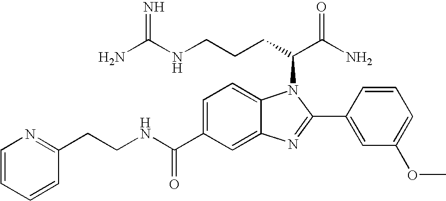

Prepared according to GP1. HPLC Rₜ: 2.69 min.

### 1-(1-amino-5-guanidino-1-oxopentan-2-yl)-2-(3-(4-chlorophenoxy)phenyl)-N-(2-(pyridin-2-yl)ethyl)-1H-benzo[d]imidazole-5-carboxamide (77)

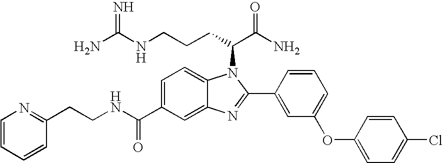

Prepared according to GP1. HPLC Rₜ: 4.13 min.

### 1-(1-amino-5-guanidino-1-oxopentan-2-yl)-N-(2-(pyridin-2-yl)ethyl)-2-(3-(trifluoromethoxy)phenyl)-1H-benzo[d]imidazole-5-carboxamide (78)

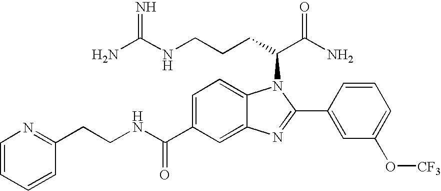

Prepared according to GP1. HPLC Rₜ: 3.43 min.

### 1-(1-amino-5-guanidino-1-oxopentan-2-yl)-2-(3-(benzyloxy)phenyl)-N-(2-(pyridin-2-yl)ethyl)-1H-benzo[d]imidazole-5-carboxamide (79)

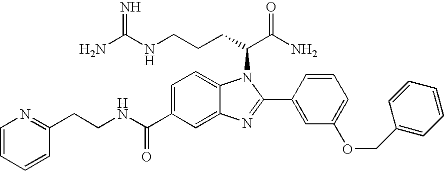

Prepared according to GP1. HPLC Rₜ: 2.79 min.

### 1-(1-amino-5-guanidino-1-oxopentan-2-yl)-2-(3-(3,4-dichlorophenoxy)phenyl)-N-(2-(pyridin-2-yl)ethyl)-1H-benzo[d]imidazole-5-carboxamide (80)

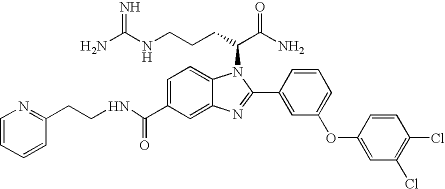

Prepared according to GP1. HPLC Rₜ: 4.42 min.

### 1-(1-amino-5-guanidino-1-oxopentan-2-yl)-N-(2-(pyridin-2-yl)ethyl)-2-(3-(3-(trifluoromethyl)phenoxy)phenyl)-1H-benzo[d]imidazole-5-carboxamide (81)

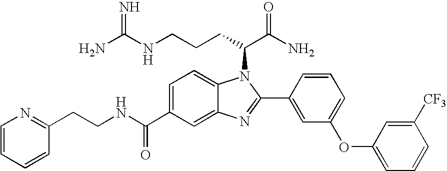

Prepared according to GP1. HPLC Rₜ: 4.33 min.

### 1-(1-amino-5-guanidino-1-oxopentan-2-yl)-2-(4-(4-methoxyphenoxy)phenyl)-N-(2-(pyridin-2-yl)ethyl)-1H-benzo[d]imidazole-5-carboxamide (82)

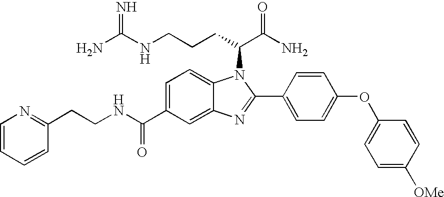

Prepared according to GP1. HPLC Rₜ: 3.80 min.

### 1-(1-amino-5-guanidino-1-oxopentan-2-yl)-2-(3-(4-tert-butylphenoxy)phenyl)-N-(2-(pyridin-2-yl)ethyl)-1H-benzo[d]imidazole-5-carboxamide (83)

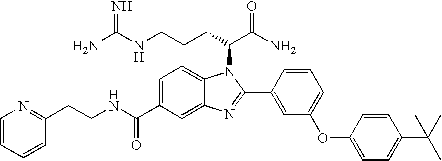

Prepared according to GP1. HPLC Rₜ: 4.78 min.

### 1-(1-amino-5-guanidino-1-oxopentan-2-yl)-2-(3-(3,5-dichlorophenoxy)phenyl)-N-(2-(pyridin-2-yl)ethyl)-1H-benzo[d]imidazole-5-carboxamide (84)

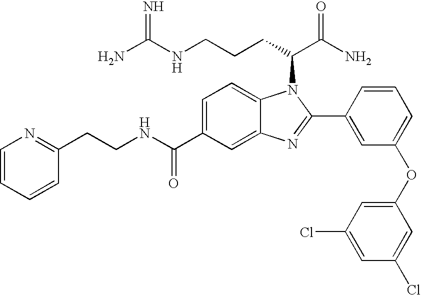

Prepared according to GP1. HPLC Rₜ: 4.49 min.

### 1-(1-amino-5-guanidino-1-oxopentan-2-yl)-2-(2-phenoxyphenyl)-N-(2-(pyridin-2-yl)ethyl)-1H-benzo[d]imidazole-5-carboxamide (85)

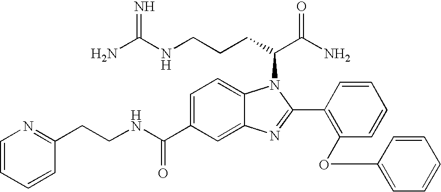

Prepared according to GP1. HPLC Rₜ: 3.48 min.

### 1-(1-amino-5-guanidino-1-oxopentan-2-yl)-2-(3-(phenylthio)phenyl)-N-(2-(pyridin-2-yl)ethyl)-1H-benzo[d]imidazole-5-carboxamide (86)

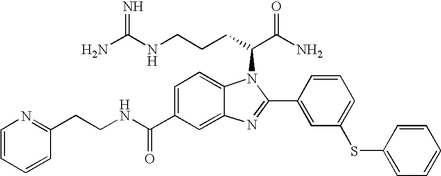

Prepared according to GP1. HPLC Rₜ: 3.89 min.

### 1-(1-amino-5-guanidino-1-oxopentan-2-yl)-2-(3-methoxyphenyl)-N-(3-(trifluoromethyl)benzyl)-1H-benzo[d]imidazole-5-carboxamide (87)

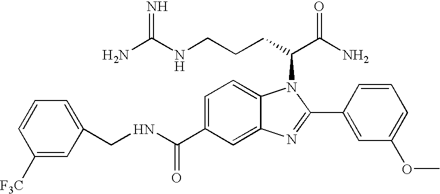

Prepared according to GP1. HPLC Rₜ: 4.13 min.

### 1-(1-amino-5-guanidino-1-oxopentan-2-yl)-2-(3-(4-chlorophenoxy)phenyl)-N-(3-(trifluoromethyl)benzyl)-1H-benzo[d]imidazole-5-carboxamide (88)

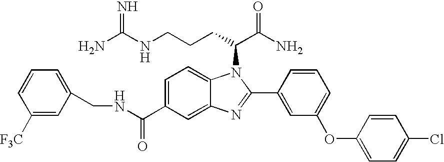

Prepared according to GP1. HPLC Rₜ: 5.13 min.

### 1-(1-amino-5-guanidino-1-oxopentan-2-yl)-2-(3-(trifluoromethoxy)phenyl)-N-(3-(trifluoromethyl)benzyl)-1H-benzo[d]imidazole-5-carboxamide (89)

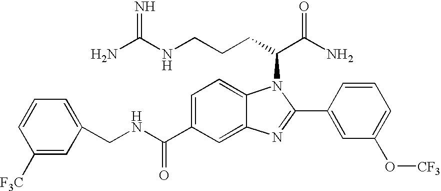

Prepared according to GP1. HPLC Rₜ: 4.62 min.

### 1-(1-amino-5-guanidino-1-oxopentan-2-yl)-2-(3-(benzyloxy)phenyl)-N-(3-(trifluoromethyl)benzyl)-1H-benzo[d]imidazole-5-carboxamide (90)

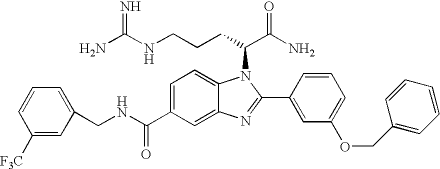

Prepared according to GP1. HPLC Rₜ: 3.91 min.

### 1-(1-amino-5-guanidino-1-oxopentan-2-yl)-2-(3-(3,4-dichlorophenoxy)phenyl)-N-(3-(trifluoromethyl)benzyl)-1H-benzo[d]imidazole-5-carboxamide (91)

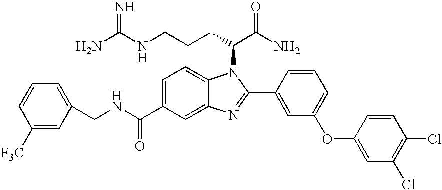

Prepared according to GP1. HPLC Rₜ: 5.34 min.

### 1-(1-amino-5-guanidino-1-oxopentan-2-yl)-N-(3-(trifluoromethyl)benzyl)-2-(3-(3-(trifluoromethyl)phenoxy)phenyl)-1H-benzo[d]imidazole-5-carboxamide (92)

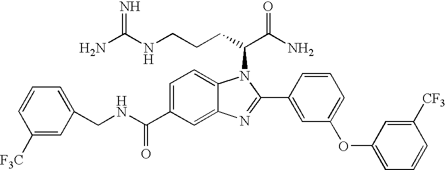

Prepared according to GP1. HPLC Rₜ: 5.24 min.

### 1-(1-amino-5-guanidino-1-oxopentan-2-yl)-2-(3-(4-methoxyphenoxy)phenyl)-N-(3-(trifluoromethyl)benzyl)-1H-benzo[d]imidazole-5-carboxamide (93)

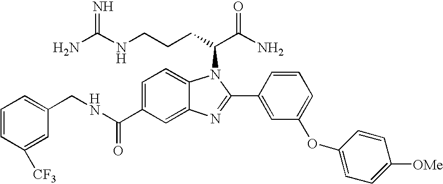

Prepared according to GP1. HPLC Rₜ: 4.86 min.

### 1-(1-amino-5-guanidino-1-oxopentan-2-yl)-2-(3-(4-tert-butylphenoxy)phenyl)-N-(3-(trifluoromethyl)benzyl)-1H-benzo[d]imidazole-5-carboxamide (94)

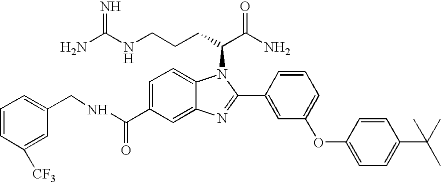

Prepared according to GP1. HPLC Rₜ: 5.66 min.

### 1-(1-amino-5-guanidino-1-oxopentan-2-yl)-2-(3-(3,5-dichlorophenoxy)phenyl)-N-(3-(trifluoromethyl)benzyl)-1H-benzo[d]imidazole-5-carboxamide (95)

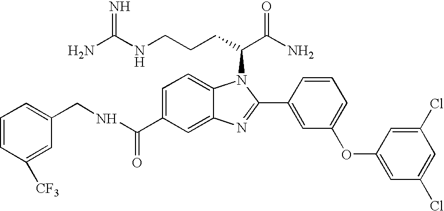

Prepared according to GP1. HPLC Rₜ: 5.45 min.

### 1-(1-amino-5-guanidino-1-oxopentan-2-yl)-2-(2-phenoxyphenyl)-N-(3-(trifluoromethyl)benzyl)-1H-benzo[d]imidazole-5-carboxamide (96)

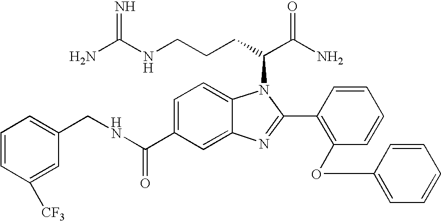

Prepared according to GP1. HPLC Rₜ: 5.4.58 min.

### 1-(1-amino-5-guanidino-1-oxopentan-2-yl)-2-(3-(phenylthio)phenyl)-N-(3-(trifluoromethyl)benzyl)-1H-benzo[d]imidazole-5-carboxamide (97)

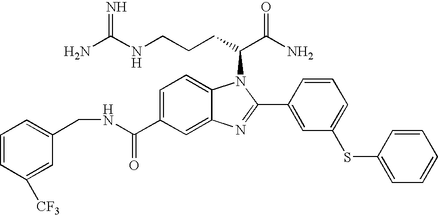

Prepared according to GP1. HPLC Rₜ: 4.82 min.

### 2-(5-(4-(4-fluorophenyl)piperazine-1-carbonyl)-2-(3-(o-tolyloxy)phenyl)-1H-benzo[d]imidazol-1-yl)-5-guanidinopentanamide (98)

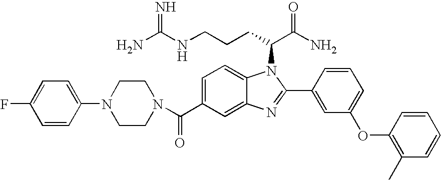

Prepared according to GP1. HPLC Rₜ: 4.91 min.

### 1-(1-amino-5-guanidino-1-oxopentan-2-yl)-N-(2-(pyridin-2-yl)ethyl)-2-(3-(o-tolyloxy)phenyl)-1H-benzo[d]imidazole-5-carboxamide (99)

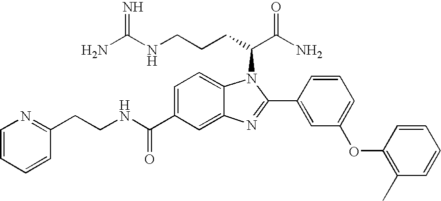

Prepared according to GP1. HPLC Rₜ: 3.94 min.

### 1-(1-amino-5-guanidino-1-oxopentan-2-yl)-2-(3-(4-fluorophenylthio)phenyl)-N-(2-(pyridin-2-yl)ethyl)-1H-benzo[d]imidazole-5-carboxamide (100)

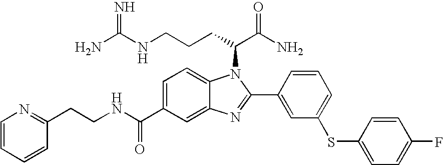

Prepared according to GP1. HPLC Rₜ: 4.01 min.

### Example 2

### Receptor Selection and Amplification Technology Assay

The functional receptor assay, Receptor Selection and Amplification Technology (R-SAT), was used to investigate the pharmacological properties of test compounds on MRGX1 or MRGX2 receptors. R-SAT is disclosed in U.S. Pat. Nos. 5,707,798, 5,912,132, and 5,955,281, each of which is incorporated by reference herein in its entirety, including any drawings.

Briefly, NIH3T3 cells were grown in 96-well tissue culture plates to 70-80% confluence. Cells were transfected for 16-20 h with plasmid DNAs using Polyfect (Qiagen Inc.) and the manufacturer's protocols. R-SATs were generally performed with 4 ng/well of receptor and 20 ng/well of β-galactosidase plasmid DNA. The human MrgX1, simian MrgX1, human MrgX2 and simian MrgX2 receptor genes were amplified by PCR from genomic DNA using oligodeoxynucleotide primers as described in U.S. patent application Ser. No. 11/359,209, which is incorporated by reference herein in its entirety, including any drawings. For large-scale transfections, cells were transfected for 16-20 h, then trypsinized and frozen in DMSO. Frozen cells were later thawed, plated at ˜10,000 cells per well of a 96 half-area well plate that contained test compound. With both methods, cells were then grown in a humidified atmosphere with 5% ambient CO₂ for five days. The medium was then removed from the plates and marker gene activity was measured by the addition of the β-galactosidase substrate o-nitrophenyl β-D-galactopyranoside (ONPG, in PBS with 0.5% NP-40). The resulting calorimetric reaction was measured in a spectrophotometric plate reader (Titertek Inc.) at 420 nm. All data were analyzed using the computer program XLFit (IDBSm). Efficacy is defined as the percent maximal activation compared to activation by a control compound (BAM22 in the case of human or simian MRGX1, STIA or Cortistatin-14 in the case of human or simian MRGX2). pEC₅₀ is the negative of log(EC₅₀), where EC₅₀ is the calculated molar concentration of test compound that produces 50% of maximum activation.

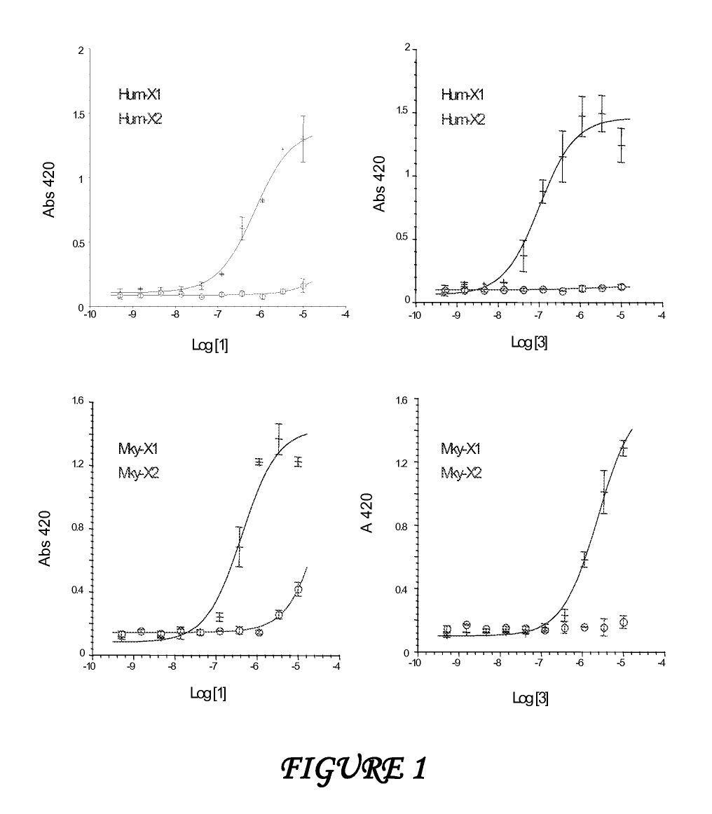

The experiments provided a molecular profile for each of the test compounds studied at the human MrgX1, simian MrgX1, human MrgX2 and simian MrgX2 receptors. As can be seen in Table 1, and FIG. 1, the compounds tested selectively activate human MrgX1, and simian MrgX1 receptors.

TABLE 1

| | Generic | Human-MrgX1 | | Human-MrgX2 | | Monkey-MrgX1 | | Monkey-MrgX2 | |
|---|---|---|---|---|---|---|---|---|---|
| ID | structure | pEC50 | Eff(%) | pEC50 | Eff(%) | pEC50 | Eff(%) | pEC50 | Eff(%) |
| 1 | Formula I | 6.0 | 146 | nr | −1 | 5.8 | 148 | <4.5 | 25 |
| 2 | Formula I | 6.2 | 220 | nr | 17 | 5.8 | 316 | nd | 22 |
| 3 | Formula I | 6.8 | 176 | nr | 4 | 5.6 | 160 | nd | 4 |
| 4 | Formula I | 6.0 | 325 | nr | 46 | 5.9 | 107 | nd | 18 |
| 27 | Formula I | 6.1 | 133 | nr | 4 | | | | |
| 30 | Formula I | 6.2 | 150 | nr | 11 | | | | |
| 31 | Formula I | 5.6 | 73 | nr | 6 | | | | |
| 32 | Formula I | 6.4 | 95 | nr | 21 | | | | |
| 33 | Formula I | 5.6 | 221 | nr | 2 | | | | |
| 34 | Formula I | 5.8 | 203 | nr | 6 | | | | |

Efficacy is reported relative to the ligands Bam22 for MrgX1 and STIA for MrgX2.

### Example 3

### MrgX1 or MrgX2 Receptor Binding Assay

Using the following materials and methods, the ability of several of the compounds disclosed herein to bind to human MrgX1, simian MrgX1, human MrgX2 and simian MrgX2 receptors can be readily determined in a receptor binding assay.

1. Grow human MrgX1, simian MrgX1, human MrgX2 and simian MrgX2 receptor gene-transfected COS cells (other transfected cell lines that do not endogenously express human MrgX1, simian MrgX1, human MrgX2 or simian MrgX2 receptors may be substituted) in a suitable growth medium in 24-well culture plates.

2. Prepare a radio-labeled assay solution by mixing 245 μL of 0.25 nM [¹²⁵I] BAM22 or Cortistatin-14 working solution with 5 μl of the following (one per solution): 50 μM unlabeled BAM22 or Cortistatin-14 working solution, 0.25 nM [¹²⁵I] BAM22 or Cortistatin-14 working solution, HEPES buffer only, or serial dilutions of the test compound.

3. Aspirate the medium from the 24-well plates using a Pasteur pipet attached to a vacuum source. Do not wash cells.

4. Add 250 μL radiolabeled assay solution from step 2 to each assay well and the plates are incubated for 60 min at room temperature (˜22° C.) on an orbital shaker at low speed.

5. Terminate the incubation by aspirating the radioactive solution with a 24-well Brandel cell harvester. The wells are washed three times with 0.5 ml ice-cold HEPES buffer using the cell harvester.

6. Aspirate the solution from the wells with a micropipettor and transfer to 12×75 mm polystyrene test tubes. Analysis is then carried out using a gamma counter (Packard, Cobra II).

7. Determine the specific binding and calculate the IC₅₀

### Example 4

### Determination of Changes in Cytosolic Calcium in Transfected HEK293 Cells

1. CHO-K1 cells transfected with human MrgX1, simian MrgX1, human MrgX2 and simian MrgX2 receptors or a control receptor at a density 1-3×10⁶ cells/mL were washed with phosphate-buffered saline.

2. Cells were loaded with 2 μM Fura-2 and analyzed with respect to the rise in intracellular calcium in the presence or absence of varying concentration of test compound.

3. The response was compared to that elicited by the application of the standard reference ligands BAM22 or Cortistatin-14 at 100 nM.

4. Intracellular free calcium concentrations were calculated using the formula:

$${[ {Ca}^{{2}^{+}} ]}_{i} = \frac{{K}_{d} ( F - {F}_{min} )}{{F}_{max} - F}$$

where K_{d} for Fura-2 is 224 nM, Fₘₐₓ is the fluorescence in the presence of 0.04% Triton-X100 and Fₘᵢₙ is the fluorescence obtained after the addition of 5 mM EGTA in 30 mM Tris-HCl, pH7.4.

Table 2 shows that the compounds tested were each active at the human MrgX1 receptors as indicated by their ability to stimulate intracellular calcium mobilization.

TABLE 2

| | |
|---|---|
| Generic | MrgX1 |

| ID | structure | pEC50 | Eff % |
|---|---|---|---|
| 2 | Formula I | 7.6 | 92 |
| 1 | Formula I | 6.6 | 88 |
| 4 | Formula I | 6.7 | 112 |
| 3 | Formula I | 6.4 | 106 |

### Example 5

### Determination of Changes in Inositol Phosphates in Transfected TsA Cells

tsA cells (a transformed HEK293 cell line) are seeded at 10,000 cells/0.1 mL per well of 96 well plates at 37° C. in a humidified 5% CO₂ incubator in DMEM supplemented with 10% fetal calf serum, penicillin (100 units/mL) and streptomycin (100 mg/mL) and grown overnight. The cells are transfected with plasmid DNAs coding receptors, or G-protein helpers when needed, using PolyFect according to the same protocol used in the RSAT as described previously. At 18-20 h post-transfection, the medium is removed and the cells are labelled overnight with 2 μCi/mL myo-[2-3H] inositol (0.1 mL/well) freshly made in the culture medium. The medium is removed and the cells are washed with Hank's Balanced Salt Solutions (HBSS) containing 1 mM CaCl₂, 1 mM MgCl₂, 20 mM LiCl and 0.1% BSA. The cells are then incubated with ligands for 45 min at 37° C. (0.1 mL/well) and the reaction is stopped by exchanging the buffer with 150 μL/well ice-cold 20 mM formic acid. 50 μL/well 0.2 M ammonium hydroxide is added and the plates are processed immediately or stored at −80° C.

To separate total [³H] inositol phosphates (IPs) ion-exchange chromatography columns are loaded with 200 μL of AG 1-X8 resin suspension (50% resin and 50% water) and the cell extracts are applied to the columns. The columns are washed with 1 mL of 40 mM ammonium hydroxide (pH 9) and eluted [³H] IPs into 2 mL deep-well blocks with 0.4 mL 2M ammonium format/0.1 M formic acid. The column is washed with 0.6 mL water. The eluates are transferred into 7 mL scintillation vials and 5 mL liquid scintillation cocktail added. The wells are mixed well and the vials are left in the dark for at least 4 h and then counted on an LS 6500 Multi-purpose Scintillation Counter (3 min/vial). This procedure collects IP1, IP2 and IP3.

### REFERENCES

The following references are referred to herein and are incorporated by reference herein in their entirety, including any drawings:
1. Dong X, Han S, Zylka M J, Simon M I, Anderson D J. A diverse family of GPCRs expressed in specific subsets of nociceptive sensory neurons. Cell. 2001 106:619-32
2. Grazzini E, Puma C, Roy M O, Yu X H, O'Donnell D, Schmidt R, Dautrey S, Ducharme J, Perkins M, Panetta R, Laird J M, Ahmad S, Lembo P M. Sensory neuron-specific receptor activation elicits central and peripheral nociceptive effects in rats. Proc Natl Acad Sci USA. 2004 May 4; 101 (18):7175-80.
3. Han S K, Dong X, Hwang J I, Zylka M J, Anderson D J, Simon M I. Orphan G protein-coupled receptors MrgA1 and MrgC11 are distinctively activated by RF-amide-related peptides through the Galpha q/11 pathway. Proc Natl Acad Sci USA. 2002 99:14740-5.
4. Hong Y, Dai P, Jiang J, Zeng X. Dual effects of intrathecal BAM22 on nociceptive responses in acute and persistent pain—potential function of a novel receptor. Br J Pharmacol. 2004 February; 141 (3):423-30.
5. Lembo P M, Grazzini E, Groblewski T, O'Donnell D, Roy M O, Zhang J, Hoffert C, Cao J, Schmidt R, Pelletier M, Labarre M, Gosselin M, Fortin Y, Banville D, Shen S H, Strom P, Payza K, Dray A, Walker P, Ahmad S. Proenkephalin A gene products activate a new family of sensory neuron—specific GPCRs. Nat. Neurosci. 2002 March; 5 (3):201-9.
6. Panula P, Aarnisalo A A, Wasowicz K. Neuropeptide F F, a mammalian neuropeptide with multiple functions. Prog Neurobiol. 1996 March-April; 48 (4-5):461-87.
7. Panula P, Kalso E, Nieminen M, Kontinen V K, Brandt A, Pertovaara A. Neuropeptide F F and modulation of pain. Brain Res. 1999 Nov. 27; 848 (1-2):191-6.
8. Reisine T and Pasternak G W (1996) Opioid analgesics and antagonists, in Goodman and Gilman's: The Pharmacological Basis of Therapeutics (Hardman J G and Limbird LE eds) pp 521-556, McGraw-Hill, New York.
9. Robas N, Mead E, Fidock M. MrgX2 is a high potency cortistatin receptor expressed in dorsal root ganglion. J Biol Chem. 2003 Nov. 7; 278 (45):44400-4
10. Zylka M J, Dong X, Southwell A L, Anderson D J. Atypical expansion in mice of the sensory neuron-specific Mrg G protein-coupled receptor family. Proc Natl Acad Sci USA. 2003 Aug. 19; 100 (17):10043-8

1. A compound of Formula I or II: 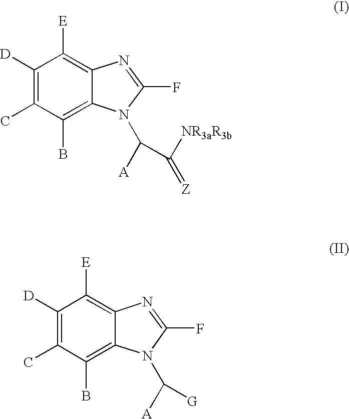
or a pharmaceutically acceptable salt or prodrug thereof, wherein:
A is selected from the group consisting of optionally substituted alkyl, optionally substituted alkenyl, optionally substituted alkynyl, optionally substituted cycloalkyl, optionally substituted (cycloalkyl)alkyl, optionally substituted cycloalkenyl, optionally substituted (cycloalkenyl)alkyl, optionally substituted aryl, optionally substituted heteroaryl, optionally substituted heteroalicyclyl, halogen, sulfenyl, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —C(R₁)NC(=Z)NR₁ₐR_{1b}, —N(R₁)C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₂, —SR₂, and —OC(=Z)R₁,
B, C and E are each independently selected from the group consisting of hydrogen, optionally substituted alkyl, optionally substituted alkenyl, optionally substituted alkynyl, optionally substituted cycloalkyl, optionally substituted (cycloalkyl)alkyl, optionally substituted cycloalkenyl, optionally substituted (cycloalkenyl)alkyl, optionally substituted aryl, optionally substituted heteroaryl, optionally substituted heteroalicyclyl, halogen, nitro, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;
D is selected from the group consisting of —C(=Z)NR₄ₐR_{4b}, —C(=Z)N(R₄)NR₄ₐR_{4b}, —C(R₄)═NR₄ₐ, —N(R₄)—C(=Z)R₄, —N(R₄)—C(=Z)NR₄ₐR_{4b}, optionally substituted heteroaryl, —C(=Z)R₄, —C(=Z)OR₄, —S(O)NR₄ₐR_{4b}, —S(O)₂NR₄ₐR_{4b}, —N(R₄)—S(═O)R₄, —N(R₄)—(S═O)₂R₄, —S(O)R₄, —S(O)₂R₄, and —OC(=Z)R₄;
F is selected from the group consisting of optionally substituted alkyl, optionally substituted alkenyl, optionally substituted alkynyl, optionally substituted cycloalkyl, optionally substituted (cycloalkyl)alkyl, optionally substituted cycloalkenyl, optionally substituted (cycloalkenyl)alkyl, optionally substituted aryl, optionally substituted heteroaryl, optionally substituted aryloxy, optionally substituted heteroaryloxy; optionally substituted heteroalicyclyl, halogen, sulfenyl, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₂, —SR₂, and —OC(=Z)R₁;
Z is selected from the group consisting of oxygen, sulfur, and NR₁;
R₁, R₁ₐ, and R_{1b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl; or R₁ₐ, and R_{1b}, taken together with the nitrogen atom or carbon atom to which the are attached, form a five- or six-membered optionally substituted cycloalkyl, optionally substituted heterocycloalkyl, optionally substituted aryl, or optionally substituted heteroaryl ring; and
R₂ is selected from the group consisting of optionally substituted alkyl, optionally substituted alkenyl, optionally substituted alkynyl, optionally substituted cycloalkyl, optionally substituted (cycloalkyl)alkyl, optionally substituted cycloalkenyl, optionally substituted (cycloalkenyl)alkyl, optionally substituted aryl, optionally substituted heteroaryl, optionally substituted heteroalicyclyl;
R₃ₐ and R_{3b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl; or R₃ₐ and R_{3b}, taken together with the nitrogen atom to which they are attached, form a five- or six-membered optionally substituted cycloalkyl, optionally substituted heterocycloalkyl, optionally substituted aryl, or optionally substituted heteroaryl ring; and
R₄, R₄ₐ, and R_{4b} are each independently selected from the group consisting of hydrogen, optionally substituted alkyl, optionally substituted alkenyl, optionally substituted alkynyl, optionally substituted amine, optionally substituted cycloalkyl, optionally substituted (cycloalkyl)alkyl, optionally substituted cycloalkenyl, optionally substituted (cycloalkenyl)alkyl, optionally substituted aryl, optionally substituted aralkyl, optionally substituted heteroaryl, optionally substituted heteroarylalkyl, optionally substituted heteroalicyclyl, wherein optionally R₄ₐ and R_{4b}, taken together with the nitrogen atom to which they are attached, form a five- or six-membered optionally substituted cycloalkyl optionally substituted heterocycloalkyl optionally substituted aryl, or optionally substituted heteroaryl ring;
wherein the alkyl, alkenyl, alkynyl, cycloalkyl, cycloalkenyl, aryl, heteroaryl, and heteroalicyclyl substituent of each of A, B, C, D, E, F, R₁, R₁ₐ, R_{1b}, R₂, R₃ₐ, R_{3b}, R₄, R₄ₐ, and R_{4b} is each independently optionally substituted with one or more substituents selected from the group consisting of halogen, R₁, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —C(R₁)NC(=Z)NR₁ₐR_{1b}, —N(R₁)C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₂, —SR₂, and —OC(=Z)R₁.

2. The compound of claim 1, wherein A is selected from the group consisting of C₁-C₈ alkyl optionally substituted with one or more —N(R₁)C(═NR₁)NR₁ₐR_{1b}.

3. The compound of claim 2, wherein R₁, R₁ₐ, R_{1b} and are hydrogen.

4. The compound of claim 1, wherein B, C, and E are each independently hydrogen.

5. The compound of claim 1, wherein D is —C(═O)NR₄ₐR_{4b}.

6. The compound of claim 5, wherein R₄ₐ and R_{4b} and is each independently selected from the group consisting of hydrogen, optionally substituted C₁-C₈ alkyl, optionally substituted C₂-C₈ alkenyl, optionally substituted C₂-C₈ alkynyl, optionally substituted amine, optionally substituted C₃-C₈ cycloalkyl, optionally substituted C₃-C₈ cycloalkenyl, optionally substituted aryl, optionally substituted ar-C₁-C₈-alkyl, optionally substituted heteroaryl, optionally substituted heteroaryl-C₁-C₈-alkyl, and optionally substituted heteroalicyclyl.

7. The compound of claim 5, wherein R₄ₐ and R_{4b} and is each independently hydrogen or optionally substituted C₁-C₈ alkyl.

8. The compound of claim 1, wherein F is selected from the group consisting of optionally substituted aryl and optionally substituted heteroaryl, wherein the aryl and heteroaryl is each independently optionally substituted with one or more substituents selected from the group consisting of halogen, R₁, nitro, —CN, —NR₁ₐR_{1b}, —N(R₁)—C(═O)R₁, and —OR₂.

9. The compound of claim 8, wherein the aryl is phenyl, which is optionally substituted with one or more substituents selected from the group consisting of chloro, bromo, methyl, ethyl, n-propyl, iso-propyl, n-butyl, sec-butyl, iso-butyl, tert-butyl, phenyl, nitro, —CN, —NH₂, —N(CH₃)₂, —NHC(═O)CH₃, —N(CH₃)C(═O)CH₃, hydroxy, methoxy, trifluoromethoxy, phenoxy, benzyloxy, and phenylmercaptyl, and wherein the phenoxy or phenylmercaptyl is each optionally substituted with one or more substituents selected from the group consisting of chloro, trifluoromethyl, methyl, ethyl, n-propyl, iso-propyl, n-butyl, sec-butyl, iso-butyl, tert-butyl, and methoxy.

10. The compound of claim 8, wherein the heteroaryl is selected from the group consisting of furan, pyrrol, imidazol, thiophene, and quinoline, and wherein the heteroaryl is optionally substituted with one or more substituents selected from the group consisting of chloro, bromo, methyl, ethyl, n-propyl, iso-propyl, n-butyl, sec-butyl, iso-butyl, tert-butyl, phenyl, nitro, —CN, —NH₂, —N(CH₃)₂, —NHC(═O)CH₃, —N(CH₃)C(═O)CH₃, hydroxyl, methoxy, and phenoxy.

11. The compound of claim 1, wherein R₁, R₁ₐ, and R_{1b} is each independently selected from the group consisting of hydrogen, optionally substituted C₁-C₈ alkyl, optionally substituted C₂-C₈ alkenyl, optionally substituted C₂-C₈ alkynyl, optionally substituted amine, optionally substituted C₃-C₈ cycloalkyl, optionally substituted C₃-C₈ cycloalkenyl, optionally substituted aryl, optionally substituted ar-C₁-C₈-alkyl, optionally substituted heteroaryl, optionally substituted heteroaryl-C₁-C₈-alkyl, and optionally substituted heteroalicyclyl.

12. The compound of claim 11, wherein R₁ₐ and R_{1b} is each independently selected from the group consisting of:
hydrogen;
aryl optionally substituted with one or more substituents selected from the group consisting of amino, methylamino, ethylamino, dimethylamino, diethylamino, methyl, ethyl, n-propyl, iso-propyl, n-butyl, sec-butyl, iso-butyl, tert-butyl, methoxy, ethoxy, fluoro, chloro, bromo, and trifluoromethyl;
C₁-C₈ alkyl optionally substituted with pyridine, pyrrolidine, pyrrolidinone, phenyl, methoxyphenyl, fluorophenyl, trifluoromethylphenyl, indazole, N-morpholine, and piperazine, and wherein the aryl is optionally substituted; and
optionally substituted heteroaryl or heteroalicyclyl.

13. The compound of claim 1, wherein R₃ₐ and R_{3b} is each independently hydrogen.

14. The compound of claim 1, wherein
A is selected from the group consisting of optionally substituted C₁-C₈ alkyl, optionally substituted C₃-C₈ cycloalkyl, optionally substituted aryl, optionally substituted heteroaryl, and —N(R₁)—C(=Z)NR₁ₐR_{1b}, wherein the alkyl, cycloalkyl, aryl, and heteroaryl is each independently optionally substituted with one or more substituents selected from the group consisting of halogen, alkoxy, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁,
B, C and E are each independently selected from the group consisting of hydrogen, optionally substituted C₁-C₈ alkyl, optionally substituted C₃-C₈ cycloalkyl, optionally substituted aryl, optionally substituted heteroaryl, and —N(R₁)—C(=Z)NR₁ₐR_{1b}, wherein the alkyl, cycloalkyl, aryl, and heteroaryl is each independently optionally substituted with one or more substituents selected from the group consisting of halogen, alkoxy, nitro, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —N(R₁)C(=Z)NR₁ₐR_{1b}, and —OC(=Z)R₁;
D is —C(=Z)NR₄ₐR_{4b};
F is selected from the group consisting of optionally substituted C₁-C₈ alkyl, optionally substituted C₂-C₈ alkenyl, optionally substituted C₂-C₈ alkynyl, optionally substituted C₃-C₈ cycloalkyl, optionally substituted C₃-C₈ cycloalkenyl, optionally substituted aryl, optionally substituted heteroaryl, optionally substituted aryloxy, optionally substituted heteroaryloxy; optionally substituted heteroalicyclyl, wherein the alkyl, alkenyl, alkynyl, cycloalkyl, cycloalkenyl, aryl, heteroaryl, and heteroalicyclyl substituent is each independently optionally substituted with one or more substituents selected from the group consisting of halogen, R₁, nitro, —CN, —NR₁ₐR_{1b}, —N(R₁)—C(=Z)R₁, —OR₂, —SR₂, and —OC(=Z)R₁;
Z is selected from the group consisting of oxygen, sulfur, and NR₁; and
R₃ₐ and R_{3b} are each independently hydrogen or optionally substituted alkyl.

15. The compound of claim 14, wherein R₃ₐ and R_{3b} are each independently hydrogen.

16. The compound of claim 1, wherein R₄ₐ, and R_{4b} are each independently selected from the group consisting of hydrogen, optionally substituted alkyl, optionally substituted cycloalkyl, optionally substituted (cycloalkyl)alkyl, optionally substituted aryl, optionally substituted aralkyl, optionally substituted heteroaryl, optionally substituted heteroarylalkyl, and optionally substituted heteroalicyclyl.

17. The compound of claim 1, wherein R₄ₐ and R_{4b} are each independently hydrogen.

18. The compound of claim 1, wherein the compound is selected from the group consisting of

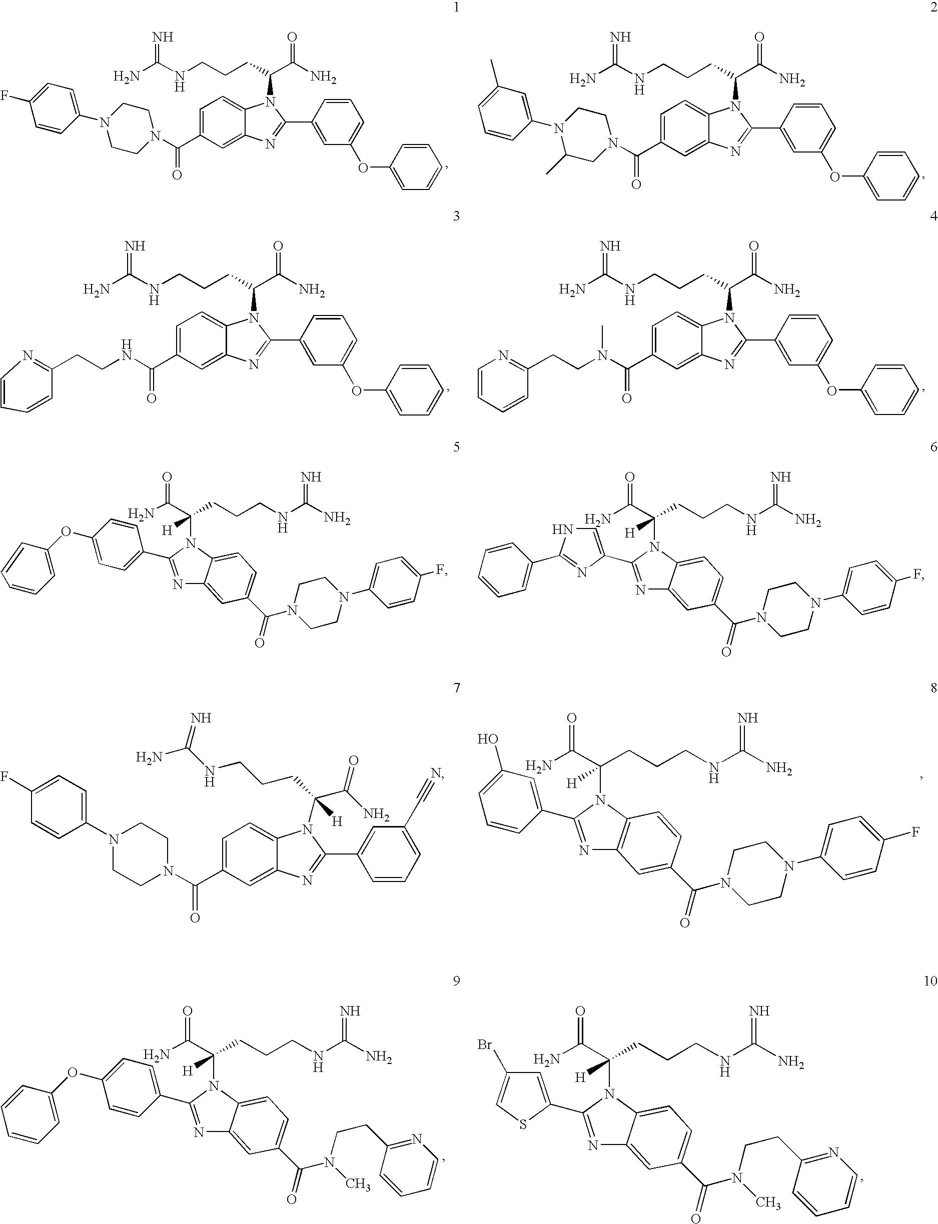

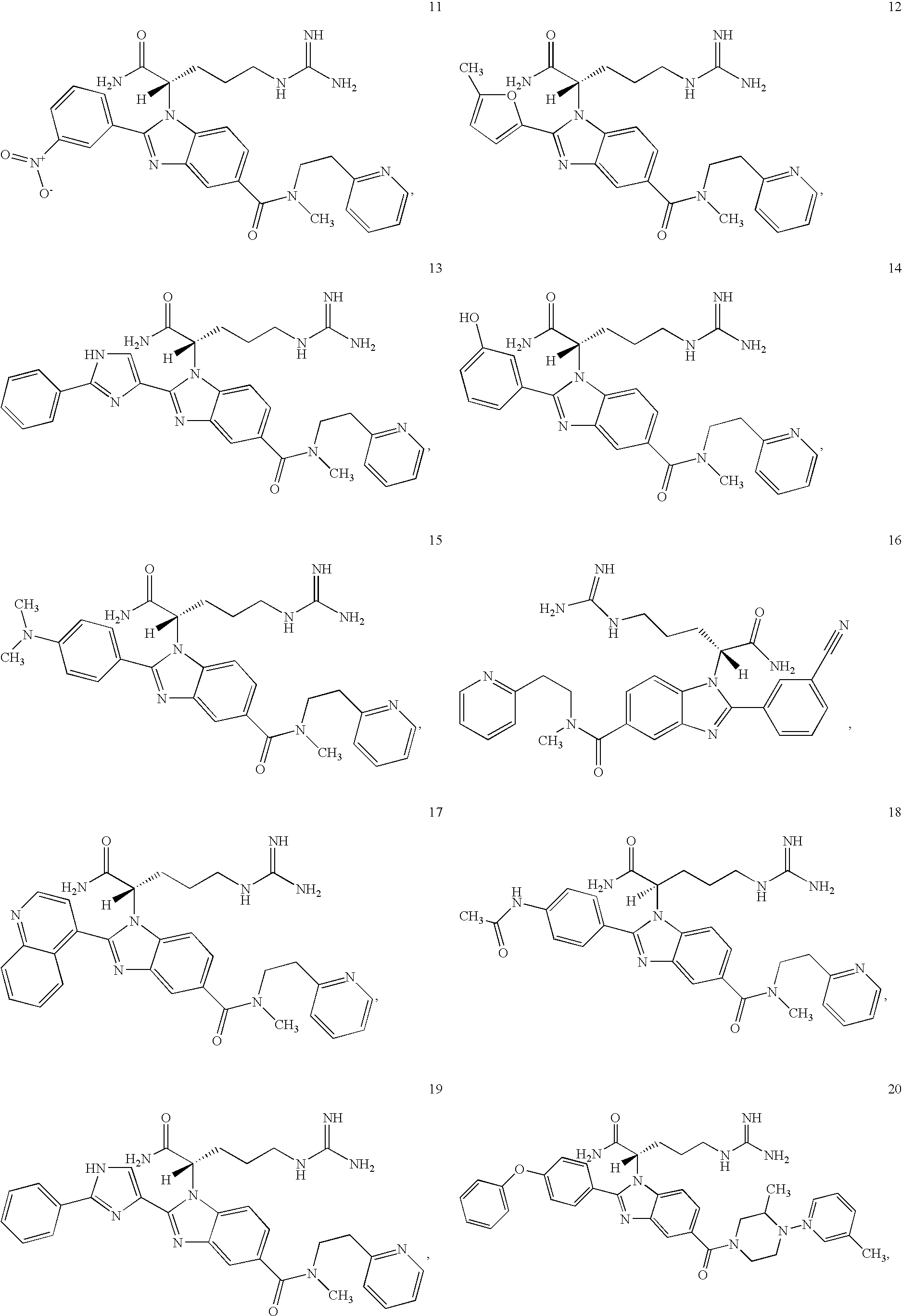

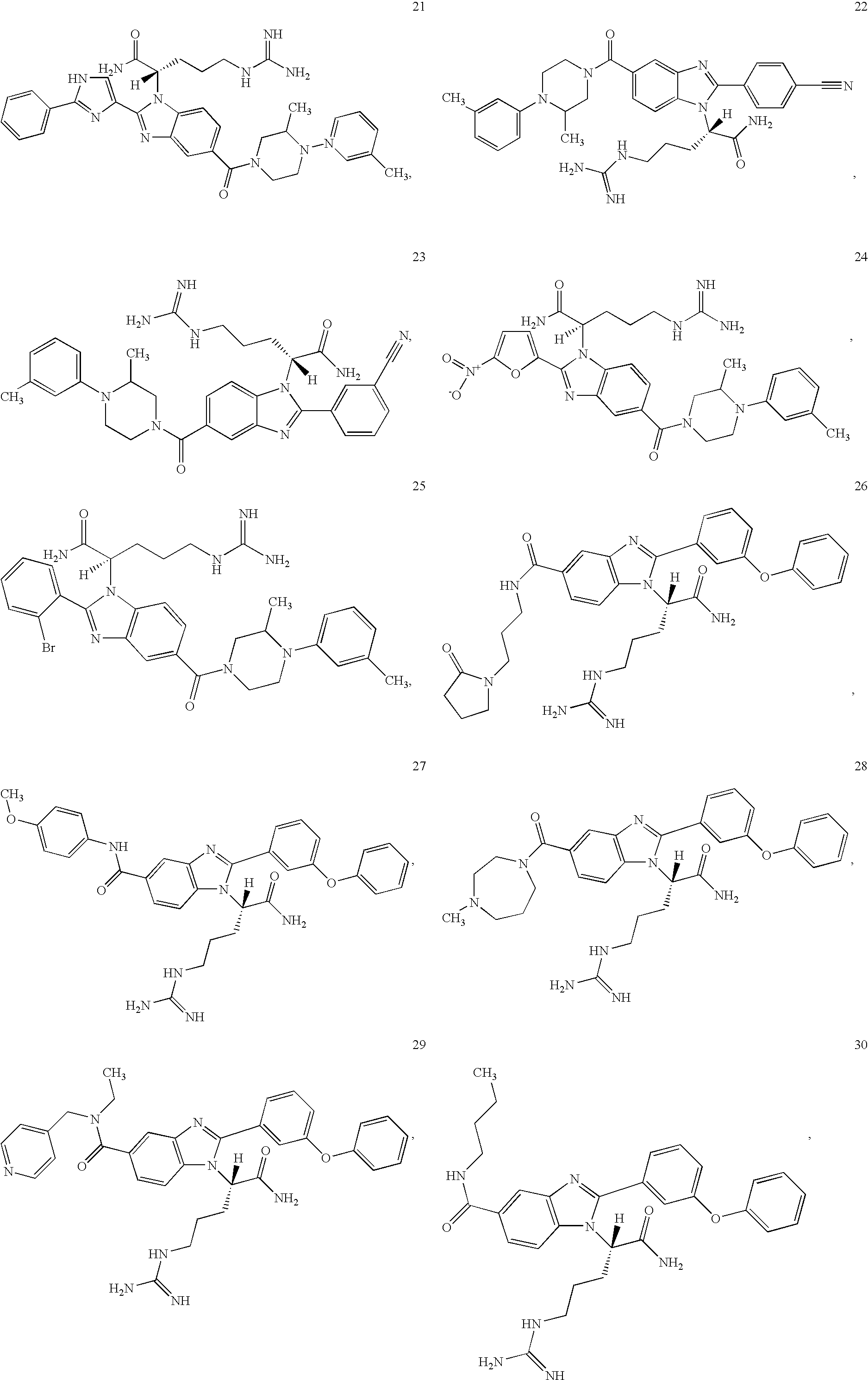

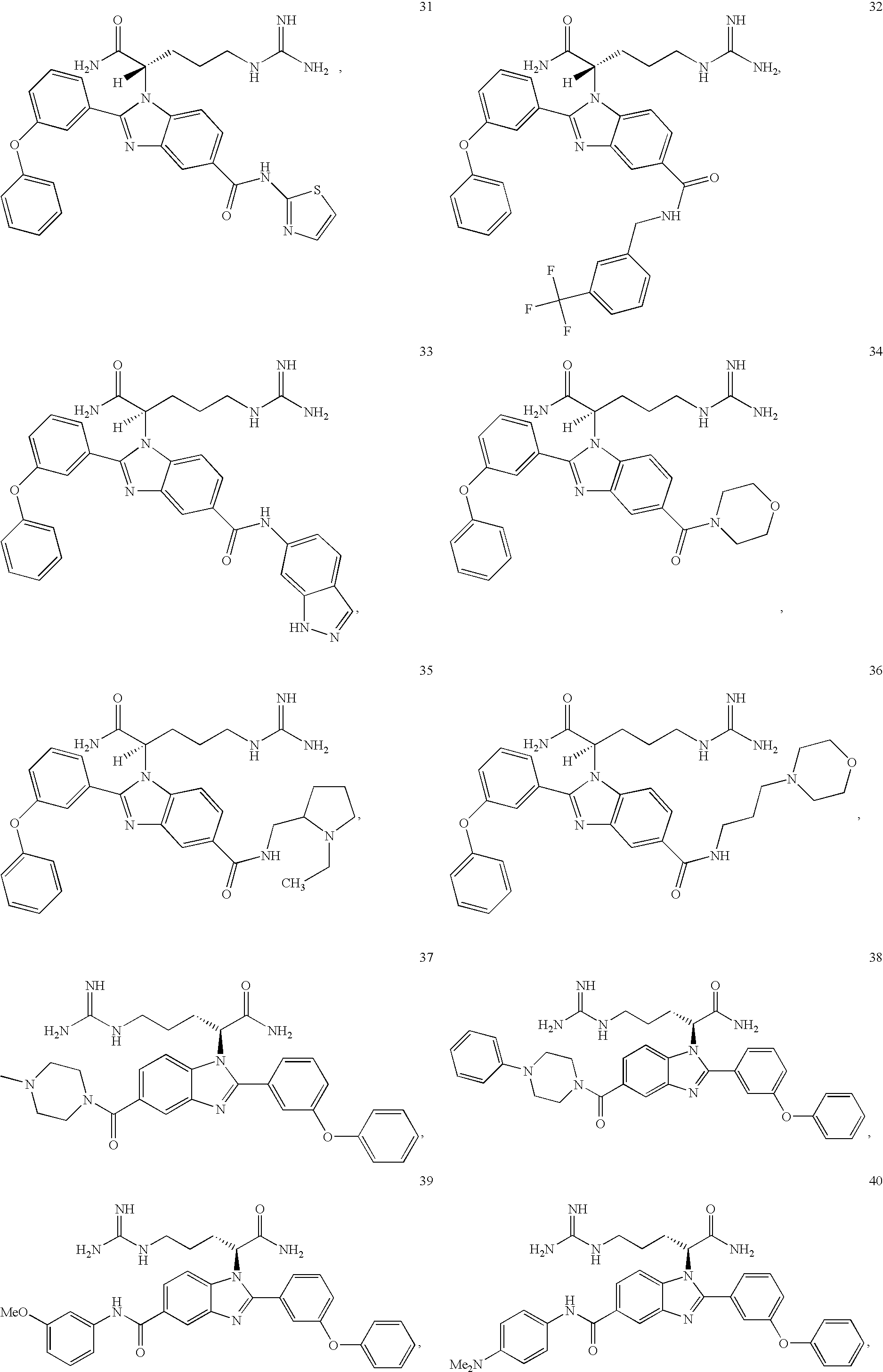

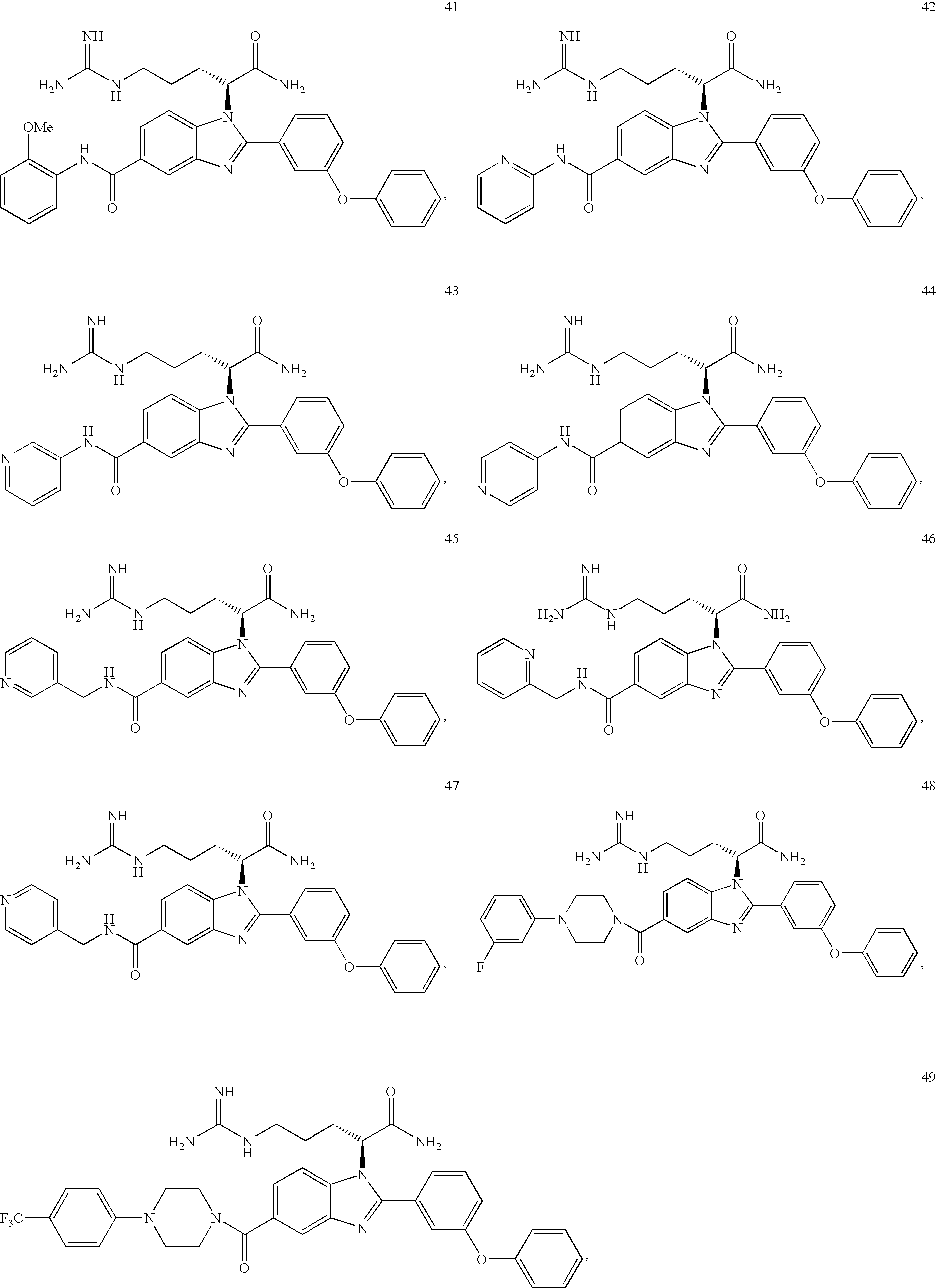

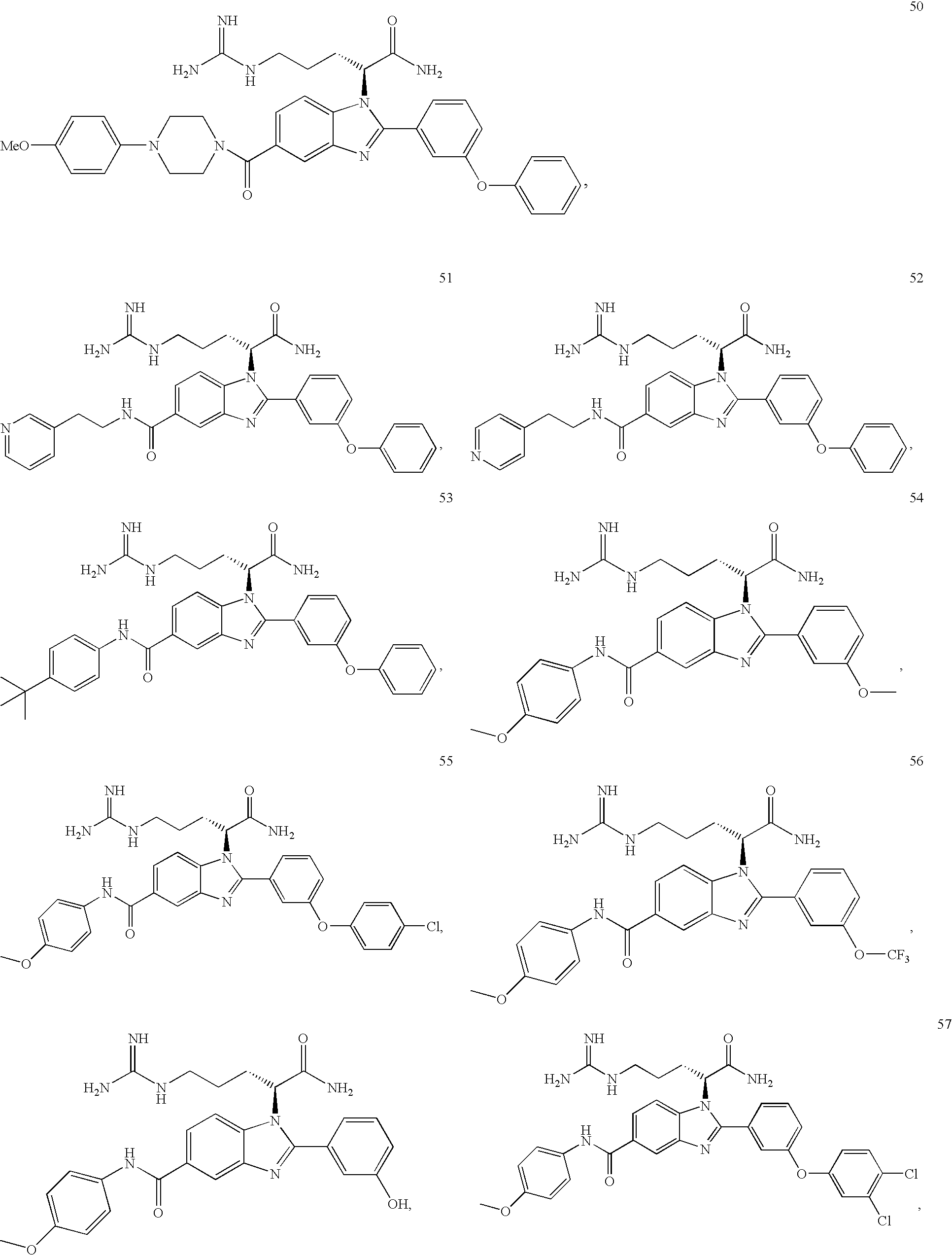

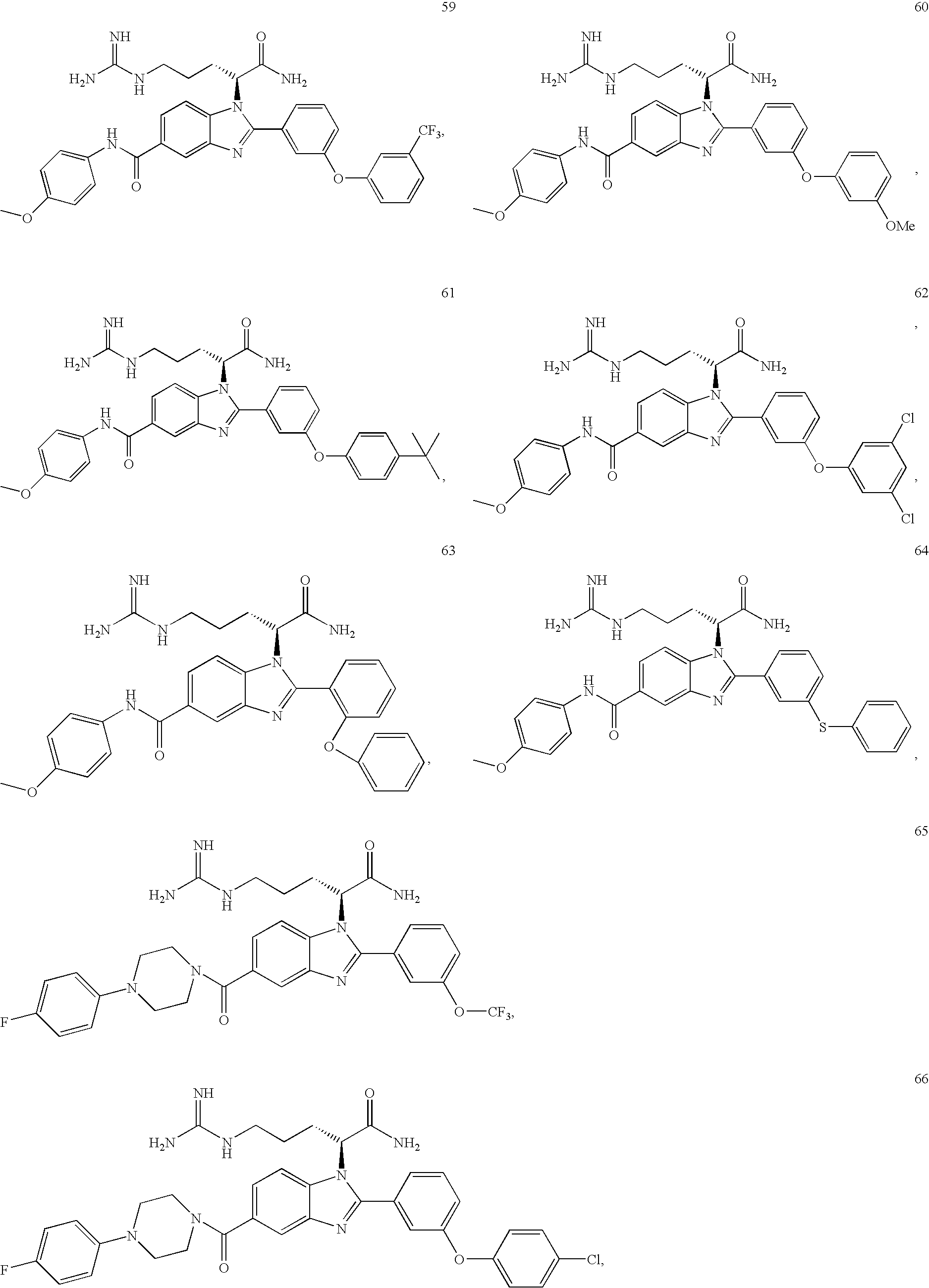

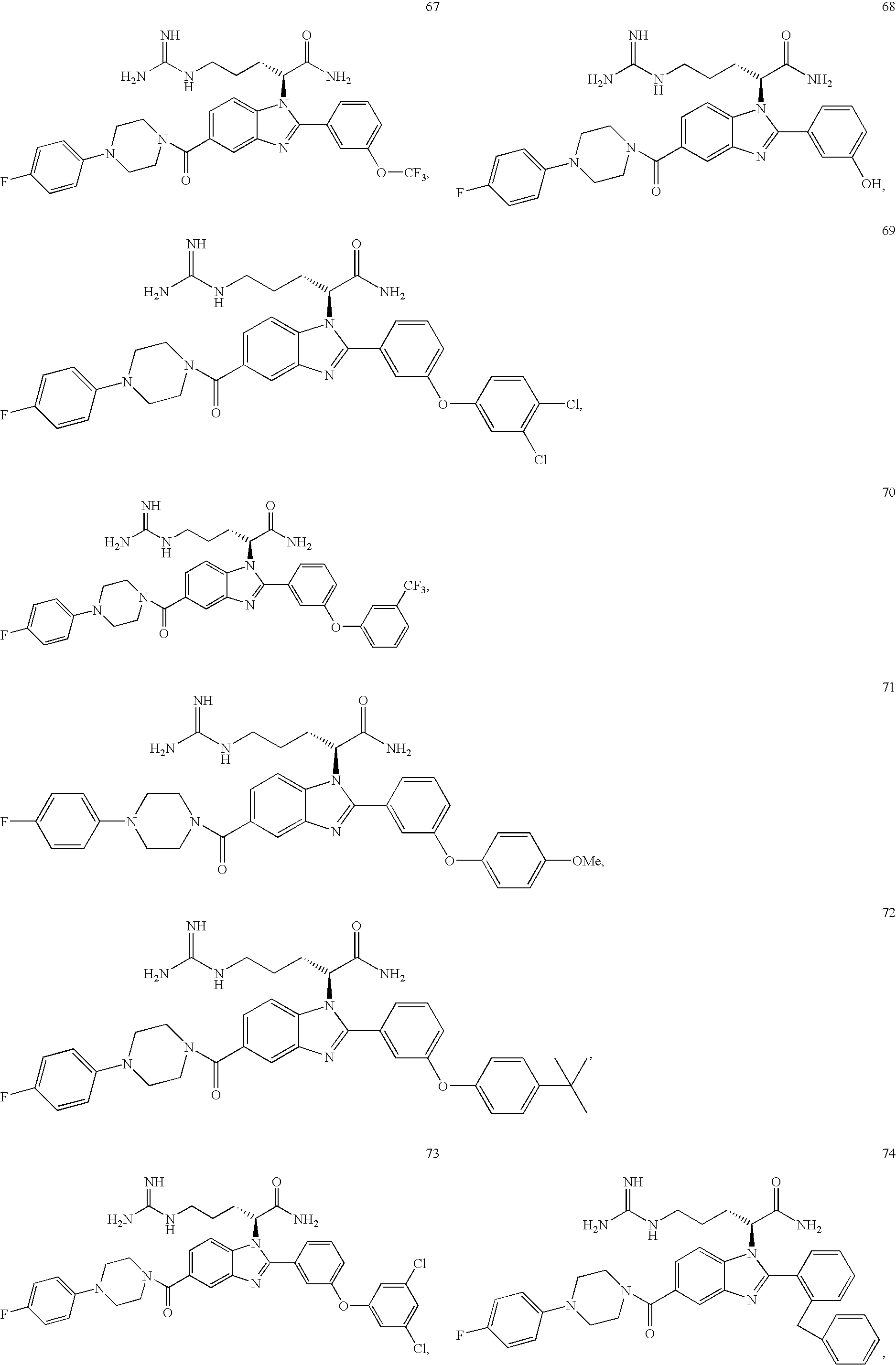

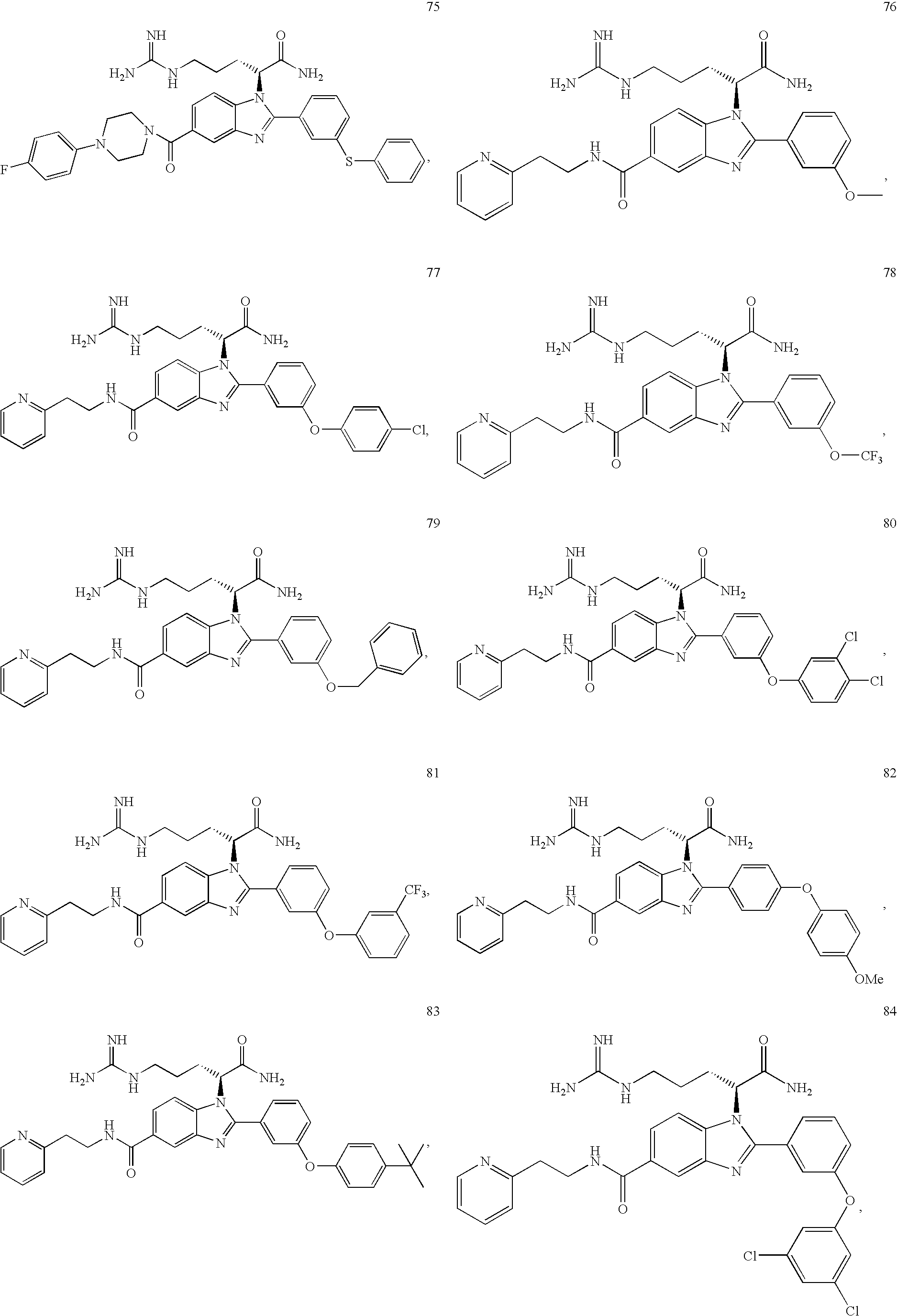

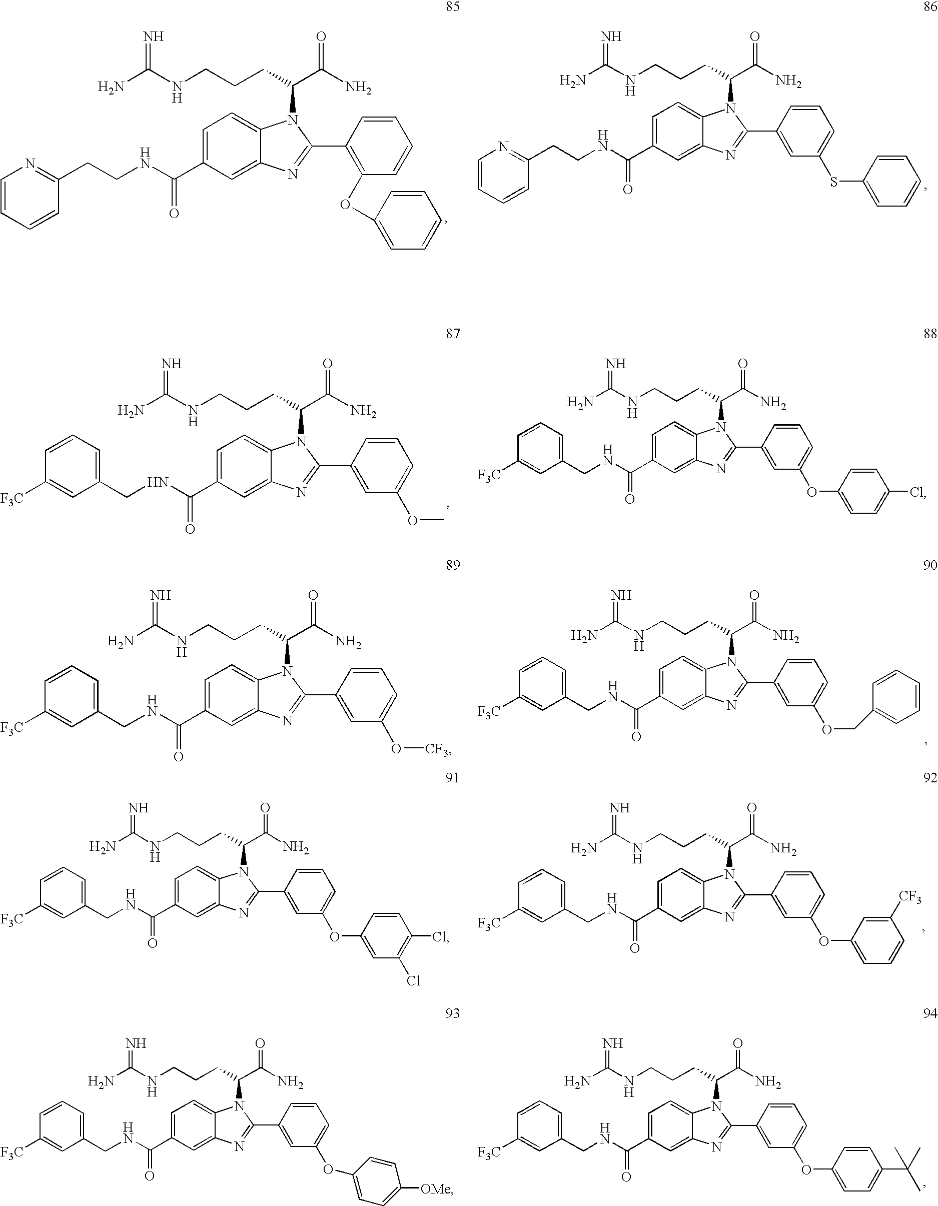

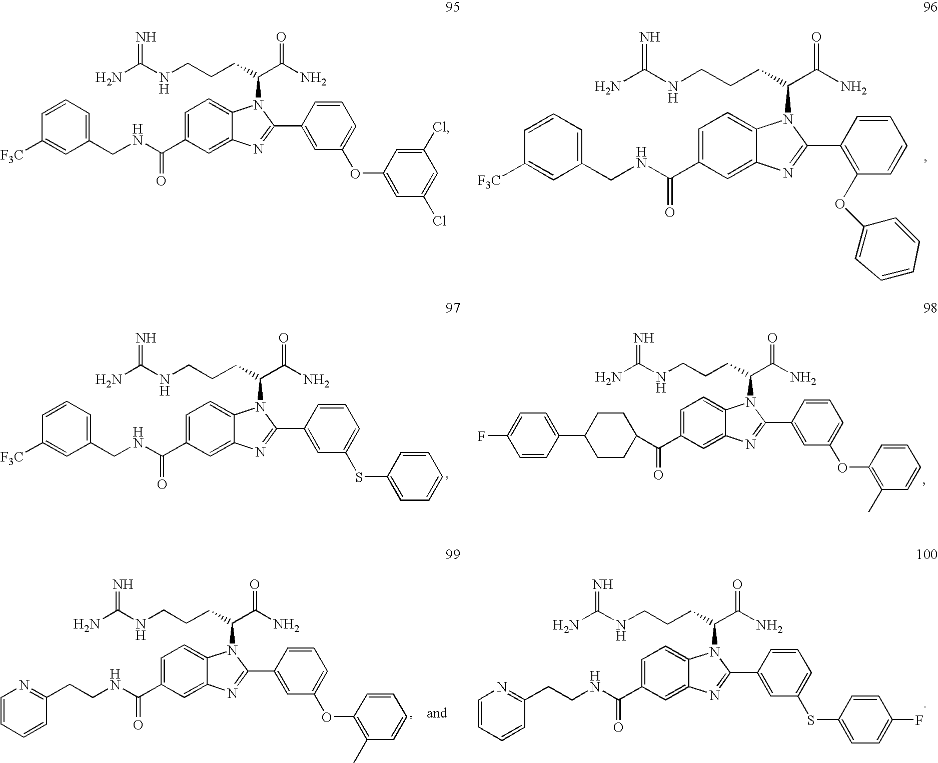

19. A pharmaceutical compositions comprising a therapeutically effective amount of one or more compound of claim 1, and a pharmaceutically acceptable carrier, excipient, or diluents.

20. (canceled)

21. The compound of claim 1, wherein R₄ₐ and R_{4b}, taken together with the nitrogen atom to which they are attached form a five- or six-membered optionally substituted cycloalkyl, optionally substituted heterocycloalkyl, optionally substituted aryl, or optionally substituted heteroaryl ring.

22. The compound of claim 21, wherein the ring is selected from the group consisting of pyridine, pyrrolidine, pyrrolidinone, indazole, N-morpholine, and piperazine.

23-34. (canceled)

35. A method of modulating the activity of an MrgX1 or an MrgX2 receptor, comprising:
contacting the MrgX1 or the MrgX2 receptor with a compound of claim 1; and
comparing the activity of the receptor after the contacting to the activity of the receptor before the contacting.

36. The method of claim 35, wherein the contacting is in vivo.

37. The method of claim 35, wherein the contacting is in vitro.

38. The method of claim 35, wherein the compound selectively modulates the MrgX1 or the MrgX2 receptor activity relative to other receptors that mediate analgesia.

39. The method of claim 38, wherein the compound selectively modulates the MrgX1 or the MrgX2 receptor relative to the opioid receptors.

40. The method of claim 35, wherein the MrgX1 or the MrgX2 receptor is selected from the group consisting of a human MrgX1 receptor, a simian MrgX1 receptor, a human MrgX2 receptor, and a simian MrgX2 receptor.

41. A method of alleviating acute, chronic and neuropathic pain in a subject, comprising:
identifying a subject in need thereof; and
administering to the subject a therapeutically effective amount of a compound of claim 1.

42. The method of claim 41, wherein the pain, whether acute pain, chronic pain or neuropathic pain, is caused by a condition selected from the group consisting of amputation, trauma, physical trauma, chemical injury, and disease.

43. The method of claim 42, wherein the disease is selected from the group consisting of diabetes, herpes zoster, irritable bowel syndrome, late-stage cancer, acute inflammation, chronic inflammation, and arthritis.

44. A method of identifying a compound that modulates the activity of an MrgX1 or an MrgX2 receptor, comprising:
contacting the MrgX1 or the MrgX2 receptor with a plurality of compounds of claim 1 one at a time;
comparing the activity of the receptor after the contacting with each compound of claim 1 to the activity of the receptor before the contacting; and
selecting a compound of claim 1 that changes the activity of the receptor after the contacting.

45. The method of claim 44, wherein the receptor is selected from the group consisting of a human MrgX1 receptor, a simian MrgX1 receptor, a human MrgX2 receptor, and a simian MrgX2 receptor.

46. The method of claim 44, wherein the receptor is located within a cell, a plurality of cells, or a cell extract that expresses the receptor.

47. A method of identifying a compound effective for the treatment of pain, comprising:
contacting a compound of claim 1 with a receptor selected from the group consisting of a human MrgX1 receptor, a simian MrgX1 receptor, a human MrgX2 receptor, and a simian MrgX2 receptor;
comparing the activity of the receptor after the contacting with each compound of claim 1 to the activity of the receptor before the contacting; and
selecting a compound of claim 1 that changes the activity of the receptor after the contacting.

48-112. (canceled)
